(19) United States
(12) Patent Application Publication
(10) Pub. No.: US 2026/0130907 A1
(43) Pub. Date: May 14, 2026

(54) ALKYLAMINE-CONTAINING SMALL MOLECULE DEGRADERS OF BCL6

(71) Applicant: DANA-FARBER CANCER INSTITUTE, INC., Boston, MA (US)

(72) Inventors: Lyn Howard JONES, Boston, MA (US); Huang HUANG, Boston, MA (US); Tinghu ZHANG, Boston, MA (US); Jianwei CHE, Boston, MA (US); Nikki KONG, Boston, MA (US); Yingpeng LIU, Boston, MA (US); Justin CRUITE, Boston, MA (US)

(73) Assignee: DANA-FARBER CANCER INSTITUTE, INC., Boston, MA (US)

(21) Appl. No.: 19/118,927

(22) PCT Filed: Oct. 16, 2023
(86) PCT No.: PCT/US2023/035198

§ 371 Date: Apr. 7, 2025

Related U.S. Application Data
(60) Provisional application No. 63/416,736, filed on Oct. 17, 2022.

(51) Int. Cl. A61K 31/506; A61K 45/06; A61P 35/00; C07D 401/14

(52) U.S. Cl. CPC A61K 31/506; A61K 45/06; A61P 35/00; C07D 401/14

(57) ABSTRACT

Described are the compounds, compositions and methods of treating a disease or disorder characterized by aberrant B-cell lymphoma 6 (BCL6) activity.

### RELATED APPLICATIONS

This application claims the benefit of priority under 35 U.S.C. § 119(e) to U.S. Provisional Application No. 63/416,736, filed Oct. 17, 2022, which is incorporated herein by reference in its entirety.

### BACKGROUND OF THE INVENTION

As demonstrated by the clinical efficacy of thalidomide analogs for the treatment of hematologic malignancies, small molecule-induced protein degradation has emerged as a powerful therapeutic strategy. Thalidomide analogs, including lenalidomide and pomalidomide, modulate the activity of the Cullin Really Interesting New Gene (RING) ligase 4-cereblon (CRBN) (CRL4^{CRBN}) E3 ubiquitin ligase to recruit and ubiquitinate neo-substrates including Ikaros family zinc finger 1 (IKZF1), IKZF3, and casein kinase 1-alpha (CK1α), which leads to their proteasomal degradation (Kronke et al., Science 343:301-305 (2014); Lu et al., Science 343:305-309 (2014); Kronke et al., Nature 523:183-188 (2015)). Other small molecules that induce protein degradation include aryl sulfonamides, which promote the destruction of RNA binding motif protein 39 (RBM39) in a CRL4-DNA damage binding protein 1 (DDB1) and CUL4 associated factor 15 (DCAF15) (CRL4^{DCAF15})-dependent manner (Han et al., Science 356:eaa13755 (2017).

Other types of small molecules include hetero-bifunctional degraders (also known as PROTACs) (Toure et al., Angew. Chem. Int. Ed. Engl. 55:1966-1973 (2016)) have been developed for a wide range of targets including kinases (Huang et al., Cell Chem. Biol. 25:88-99 (2018)), nuclear receptors (Bondeson et al., Nat. Chem. Biol. 11:611-617 (2015)), and epigenetic enzymes (Winter et al., Science 348:1376-1381 (2015)). These small molecule degraders engage both the E3 ligase and the target protein substrate, promoting formation of a substrate-drug-ligase ternary complex (Nowak et al., Nat. Chem. Biol. 14:706-714 (2018); Petzold et al., Nature 532:127-130 (2016); Sievers et al., Science 362:aat0572 (2018)).

While degraders can show remarkable efficacy and sustained target depletion, some putative target proteins have proven recalcitrant to this approach. One such example is the B cell lymphoma 6 (BCL6) protein, for which hetero-bifunctional degraders have shown insufficient target modulation to induce growth inhibition (McCoull et a/., ACS Chem. Biol. 13:3131-3141 (2018)).

BCL6 was first identified as a locus affected by chromosomal translocations in diffuse large B-cell lymphomas (DLBCL). It is now known to be broadly expressed in many lymphomas.

Its role in lymphomagenesis stems from its function in the humoral immune system, where upregulation of BCL6 is required for the formation of germinal centers (GC) during the humoral immune response (Ye et al., Nat. Genet. 16:161-170 (1997); Dent et al., Science 276:89-92 (1997)). GCs are transient structures that form in response to antigen stimulation. Within GCs, B cells tolerate massive proliferation and the mutagenic effect of the DNA-editing enzyme AICDA to undergo immunoglobulin affinity maturation (Klein et al., Nat. Rev. Immunol. 8:22-33 (2008)).

These activities are orchestrated by and dependent on BCL6, a powerful transcriptional repressor that silences hundreds of genes. Some of these target genes control DNA damage sensing (i.e., ATR, CHEK1, TP53, ARF) and proliferation checkpoints (i.e., CDKN1A, CDKN1B, CDKN2A, CDKN2B, PTEN) (Hatzi et al., Trends Mol. Med. 20:343-352 (2014)). BCL6 also represses genes required for exit from the GC reaction and plasma cell differentiation (e.g., JRF4, PRDM1). This ensures that GC B cells have sufficient time to acquire somatic hyper-mutation of their immunoglobulin genes. Thus, deregulated suppression of these target genes could result in malignant transformation of B cells.

BCL6 also represses numerous oncogenes in GC B cells, including MYC, BCL2, BMI1, and CCND1 (Ci et al., Blood 113:5536-5548 (2009)). Through this function, BCL6 may mitigate its own pro-oncogenic checkpoint repression effect and thus reduce the potential for malignant transformation of GC B cells. This effect is abrogated in the presence of BCL2 or MYC translocations, which drive expression of these oncogenes through aberrant regulatory elements. The presence of both MYC and/or BCL2 together with BCL6 (regardless of translocations) is clearly deleterious because it provides B cells with simultaneous suppression of checkpoints through BCL6, along with the pro-growth and survival effects of MYC and BCL6 (Cardenas et al., Clin. Cancer Res. 23:885-893 (2017)). In the normal immune response, BCL6 function is terminated by the disruption of BCL6 transcriptional complexes through CD40-induced ERK signaling and downregulation of BCL6 mRNA by IRF4 and PRDM1 (Polo et al., Blood 112:644-651 (2008)). Termination of BCL6 function is required for B cells to exit the GC reaction.

BCL6 is a promising drug target for non-Hodgkin lymphomas such as diffuse large B cell lymphoma (DLBCL) (Cerchietti et al., Cancer Cell 17:400-411 (2010); Cardenas et al., J. Clin. Invest. 126:3351-3362 (2016)) and follicular lymphoma (Bosga-Bouwer et al., Genes Chromosomes Cancer 44:301-304 (2005)). Pathologically increased BCL6 expression, as a result of somatic BCL6 translocation, exonic mutation, promoter mutation, or mutations in regulatory pathways, is a common driver of B cell malignancies (Hatzi et al., Trends Mol. Med. 20:343-352 (2014)). In genetically engineered mice, overexpression of BCL6 is sufficient to drive lymphoma development (Cattoretti et al., Cancer Cell 7:445-455 (2005)). BCL6 acts as a master transcriptional repressor enabling rapid expression of germinal center (GC) B cells and tolerance to genomic instability caused by hypermutation of the immunoglobulin genes and class switch recombination (Hatzi et al., Trends Mol. Med. 20:343-352 (2014)). BCL6 represses a broad range of genes involved in the DNA damage response (Ranuncolo et al., Blood Cells Mol. Dis. 41:95-99 (2008)), cell cycle checkpoints (Tunyaplin et al., J. Immunol. 173:1158-1165 (2004)), and differentiation (Phan et al., Nat. Immunol. 6:1054-1060 (2005)). As expected, knock-out of BCL6 in lymphoma cells results in tumor stasis (Schlager et al., Oncotarget 11:875-890 (2020)). Several peptide and small molecule inhibitors targeting BCL6 have shown efficacy in vivo, but only at high concentrations, which has limited their translation into clinical therapeutic agents (Cerchietti et al., Cancer Cell 17:400-411 (2010); Cardenas et al., J. Clin. Invest. 126:3351-3362 (2016)).

Broad complex/Tramtrack/Bric-a-brac (BTB) proteins are a diverse family of proteins characterized by the presence of a common protein-protein interaction domain, known as the BTB domain. BTB proteins have diverse functions ranging from transcriptional regulation and chromatin remodeling to protein degradation and cytoskeletal regulation. Specificity of function is determined in part by additional domains present in a given BTB protein, as well as by interaction partners. Studies of BTB proteins in Drosophi/a and mammalian systems have revealed the importance of these proteins in multiple developmental contexts, as well as in cancer and neurological and musculoskeletal diseases. BTB proteins play critical roles in transcriptional regulation and chromatin remodeling (Chaharbakhshi et al., Genesis 54:505-518 (2016)).

The BTB domain mediates various functions of BCL6, such as homodimerization and interaction with co-repressor proteins (Ghetu et al., Mol. Cell 29:384-391 (2008); Ahmad et al., Mol. Cell 12:1551-1564 (2003)). Techniques that disrupt the protein-protein interaction between the BTB domain of BCL6 and its co-repressors may be useful to combat BCL6-related diseases.

### SUMMARY OF THE INVENTION

A first aspect of the present invention is directed to a compound having a structure represented by formula (I):

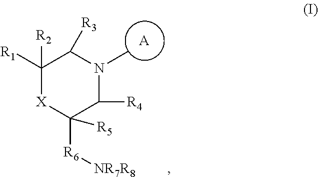

or a pharmaceutically acceptable salt or stereoisomer thereof,
wherein:
X is CH₂, S, CHF, CHCl, CHOH, or CF₂;
R₁ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
   each R₉ is independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups;
   each R₁₀ is independently alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups, or
R₁ and R₂ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₁₀ groups;
R₃ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₄ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₅ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₆ is absent, (C₁-C₆)alkylene, or (C₃-C₇)carbocyclyl; wherein said alkylene or carbocyclyl, is further optionally substituted by one or more, identical or different R₁₀ groups, or
R₅ and R₆ together with the same carbon atom to which they are attached form a spiro (C₃-C₇)carbocyclyl group or a 4- to 7-membered heterocyclyl group; wherein said carbocyclyl or heterocyclyl, is further optionally substituted by one or more, identical or different R₁₀ groups, or
R₆ is (C₂-C₄)alkylene which is bound to R₇ to form a 4- to 6-membered heterocyclyl group;
R₇ and R₈ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups, or
R₇ and R₈ together with the nitrogen atom to which they are attached form a 3- to 7-membered heterocyclyl, wherein said heterocyclyl is further optionally substituted by one or more, identical or different R₁₀ groups;

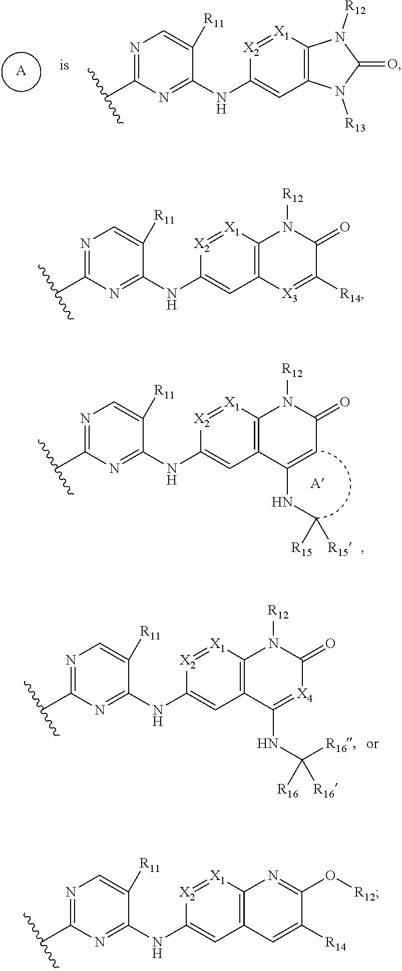

X₁ and X₂ are independently CR₁₇, or N;
   R₁₇ is hydrogen, (C₁-C₄)alkyl, halo, hydroxy, amino, (C₁-C₄)alkoxy, (C₁-C₄)haloalkyl, (C₁-C₄)haloalkoxy, (C₂-C₄)alkenyl, (C₂-C₄)alkynyl, nitro, cyano, NH(C₁-C₄)alkyl, or N(C₁-C₄ alkyl)₂;
X₃ is CH or N;
X₄ is CR_{17′}, or N;
   R_{17′} is hydrogen, fluor, chloro, or methyl;
R₁₁ is Cl or CN;
R₁₂ is hydrogen, (C₁-C₆)alkyl, (C₃-C₆)carbocyclyl, 4- to 7-membered heterocyclyl, (C₃-C₇)carbocyclyl(C₁-C₆)alkyl, or 4- to 7-membered heterocyclyl(C₁-C₆)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl, or
R₁₂ is -L-Y—Z or -L-Y—Z;
   L is absent or (C₁-C₅)alkylene optionally substituted by one or more substituents selected from (C₁-C₂)alkyl and oxo;
   Y is absent, O, S, S(O), S(O)₂, NR′, C(O), C(O)O, OC(O), C(O)N(R′), N(R′)C(O), N(R′)C(O)N(R′), N(R′)C(O)O, OC(O)N(R′), S(O)₂N(R′), or N(R′)S(O)₂;
   each R′ is independently hydrogen or (C₁-C₄)alkyl;
Z is hydrogen, (C₁-C₆)alkyl, (C₂-C₆)alkenyl, (C₂-C₆)alkynyl, (C₃-C₁₀)carbocyclyl, or 3- to 10-membered heterocyclyl; wherein Z is optionally substituted by one or more substituents independently selected from (C₁-C₄)alkyl, halo, (C₁-C₄)haloalkyl, (C₁-C₄)haloalkoxy, amino, (C₁-C₄)aminoalkyl, cyano, hydroxy, carboxy, carbamoyl, sulphamoyl, mercapto, ureido, NR^{r}R^{s}, OR^{r}, C(O)R^{r}, C(O)OR^{r}, OC(O)R^{r}, C(O)NR^{r}R^{s}, N(R^{r})C(O)R^{r}, S(O)₀₋₂R^{r}, S(O)₂NR^{r}R^{s}, N(R)SO₂R^{r}, Si(R^{r})(R^{s})R^{t} and (CH₂)₁₋₃NR^{r}R^{s}; wherein R^{r}, R^{s}, and R^{t} are each independently hydrogen, (C₁-C₆)alkyl, or (C₃-C₆)cycloalkyl; or R^{r} and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, and hydroxy;
R₁₃ is hydrogen, methyl, —(CH₂)₁₋₃W₁W₂, or

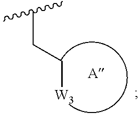

W₁ is CR₁₈R_{18′} or C(O);
   R₁₈ and R_{18′} are independently hydrogen, (C₁-C₂)alkyl, fluoro, hydroxy, cyano, nitro, (C₁-C₂)alkoxy, (C₁-C₂)haloalkyl, (C₁-C₂)haloalkoxy, amino, NH(C₁-C₂)alkyl, or N(C₁-C₂ alkyl)₂, or
R₁₈ and R_{18′} together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
W₂ is cyano, hydroxy, 5- or 6-membered heteroaryl, phenyl, C(O)—(C₁-C₂)alkyl, S(O)₂—(C₁-C₂)alkyl, C(O)OCH₃, C(O)NHCH₃, CR₁₉R₂₀R₂₁, amino, NH(C₁-C₂)alkyl, or N(C₁-C₂ alkyl)₂:
   R₁₉ is hydrogen, (C₁-C₂)alkyl, fluoro, chloro, bromo, hydroxy, amino, cyano, nitro, (C₁-C₂)alkoxy, (C₁-C₂)haloalkyl, or (C₁-C₂)haloalkoxy;
   R₂₀ is hydrogen, (C₁-C₂)alkyl, fluoro, chloro, bromo, hydroxy, cyano, nitro, (C₁-C₂)alkoxy, (C₁-C₂)haloalkyl, (C₁-C₂)haloalkoxy, or —Y₂-L₂-Z₂;
      Y₂ is absent, O, S, S(O), S(O)₂, NR′, C(O), C(O)O, OC(O), C(O)N(R′), N(R′)C(O), S(O)₂N(R′), or N(R′)SO₂;
      L₂ is absent or (C₁-C₂)alkylene;
      Z₂ is hydrogen, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, hydroxy, C(O)R′, C(O)OR′, OC(O)R′, C(O)NR′R′, and N(R′)C(O)R′, wherein each R′ is independently hydrogen or (C₁-C₄)alkyl;
or R₁₉ and R₂₀ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
   R₂₁ is (C₁-C₂)alkyl, —C(O)OR″, OR″, —C(O)NR″, NR″R″, phenyl, or 5-membered heteroaryl, wherein each R″ is independently hydrogen or (C₁-C₂)alkyl;
   A″ is (C₄-C₆)carbocyclyl or 4- to 6-membered heterocyclyl, optionally substituted with one or more substituents independently selected from (C₁-C₂)alkyl, halo, hydroxy, cyano, and (C₁-C₂)alkoxy;
   W₃ is NR₂₂ or CR₂₃R_{23′};
      R₂₂ is hydrogen, (C₁-C₂)alkyl, (C₁-C₄)haloalkyl, (C₁-C₄)hydroxyalkyl, —C(O)CH₃, or —C(O)O—(C₁-C₄)alkyl;
      R₂₃ and R_{23′} are independently hydrogen, (C₁-C₂)alkyl, cyclopropyl, fluoro, chloro, bromo, hydroxy, amino, cyano, nitro, (C₁-C₂)haloalkyl, (C₁-C₂)haloalkoxy, (C₁-C₂)alkoxy, —C(O)OR″, NR″R″, phenyl, or 5-membered heteroaryl;
R₁₄ is -L₃CR₂₄R₂₅R₂₆, or —CH═CH—R₂₆;
   L₃ is absent, O, S, (C₁-C₄)alkylene, —O—(C₁-C₄)alkylene, or —S—(C₁-C₄)alkylene;
   R₂₄ is hydrogen or (C₁-C₄)alkyl;
   R₂₅ is hydrogen or (C₁-C₄)alkyl, or
   R₂₄ and R₂₅ together with the carbon atom to which they are attached form a (C₃-C₅)carbocyclyl, 4- to 7-membered heterocyclyl, or C═O;
   R₂₆ is (C₁-C₆)alkyl, —NR₂₇R₂₈, —OR₂₇, —C(O)R₂₇, —C(O)OR₂₇, —N(R₂₈)C(O)R₂₇, —C(O)NR₂₇R₂₈, —S(O)—(C₁-C₆)alkyl, —S(O)₂—(C₁-C₆)alkyl, —P(O)—(C₁-C₆ alkyl)₂, —C(NH)NH₂, or —(C₁-C₄)alkyl-NR₂₈C(O)R₂₇;
      R₂₇ is hydrogen, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
      R₂₈ is hydrogen or (C₁-C₄)alkyl;
R₁₅ is hydrogen, (C₁-C₄)alkyl, (C₃-C₆)cycloalkyl, (C₁-C₄)haloalkyl, or cyano, wherein said alkyl or cycloalkyl is optionally substituted by one or more substituents selected from (C₁-C₄)alkyl, (C₃-C₆)cycloalkyl, hydroxy, (C₁-C₂)alkoxy, amino, NH(C₁-C₂)alkyl, N((C₁-C₂)alkyl)₂, (C₁-C₂)aminoalkyl, and halo;
R₁₅′ is hydrogen, (C₁-C₄)alkyl, cyano, (C₁-C₄)haloalkyl, or —Y₃-L₄-Z₃;
   Y₃ is absent, C(O)O, or C(O)N(R′);
   L₄ is absent or (C₁-C₂)alkylene;
   Z₃ is hydrogen, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, amino, nitro, cyano, and hydroxy, or
R₁₅ and R₁₅′, together with the carbon atom to which they are attached, form a (C₄-C₆)carbocyclyl, or 4- to 6-membered heterocyclyl;
A′ is a 6- or 7-membered heterocyclyl, which in addition to R₁₅ and R₁₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, amino, cyano, and hydroxy;
R₁₆ is hydrogen, (C₁-C₂)alkyl, (C₃-C₄)cycloalkyl, (C₁-C₂)haloalkyl, cyano, (C₂-C₄)alkenyl, or (C₂-C₄)alkynyl;
R¹⁶′ is (C₁-C₄)alkyl, cyano, (C₁-C₄)haloalkyl, or —Y₄-L₅-Z₄;
   Y₄ is absent, C(O), C(O)O, OC(O), C(O)N(R′), or S(O)₂N(R′);
   L₅ is absent or (C₁-C₂)alkylene optionally substituted by one or more substituents selected from (C₁-C₂)alkyl and oxo;
   Z₄ is hydrogen, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, amino, nitro, cyano, hydroxy, C(O)R^{u}, C(O)OR^{u}, OC(O)R^{u}, C(O)NR^{u}R^{u}, and N(R^{u})C(O)R^{u}, wherein each R^{u} is independently hydrogen, (C₁-C₄)alkyl, or (C₃-C₆)cycloalkyl, or
   Z₄ is -Q-L₆-W, wherein
      Q is absent, O, NH, or N(C₁-C₂)alkyl;
      L₆ is absent or (C₁-C₂)alkylene optionally substituted by one or more substituents selected from oxo and (C₁-C₂)alkyl;
   W is (C₁-C₄)alkyl, phenyl, (C₃-C₆)cycloalkyl, (C₃-C₆)cycloalkenyl, or 5- or 6-membered heterocyclyl, wherein W is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, nitro, cyano, or hydroxy, or
R₁₆ and R₁₆′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; and
R₁₆″ is hydrogen, (C₁-C₄)alkyl, (C₁-C₂)haloalkyl, (C₁-C₂)alkoxy, (C₁-C₂)haloalkoxy, cyano, nitro, acetylenyl, phenyl, or 5- or 6-membered heteroaryl, wherein said alkyl, phenyl, or heteroaryl is optionally substituted by one or more substituents independently selected from halo, hydroxy, and amino;
   provided that when R₇ is (C₁-C₃)alkyl and R₈ is (C₁-C₃)alkyl, R₆ is not (C₁-C₆)alkylene, and provided that when

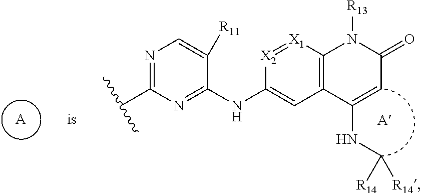

R₁₁ is Cl, X is CH₂ or CF₂, and R₆ is absent or optionally substituted (C₁-C₂)alkylene, R₇ and R₈ cannot be a) independently hydrogen or (C₁-C₂)alkyl optionally substituted with an oxo group, or b) R₇ and R₈ together with the nitrogen atom to which they are attached form a 4-membered heterocyclyl optionally substituted with a gem-difluoro group.

Another aspect of the present invention is directed to a pharmaceutical composition that includes a therapeutically effective amount of a compound of formula (I) or a pharmaceutically acceptable salt or stereoisomer thereof, and a pharmaceutically acceptable carrier.

A further aspect of the present invention is directed to a method of treating a disease or disorder that is characterized or mediated by aberrant BCL6 activity that entails administering to a subject in need thereof a therapeutically effective amount of a compound of formula (I) or a pharmaceutically acceptable salt or stereoisomer thereof.

In some embodiments, the disease or disorder is a lymphoid malignancy. In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma. In some embodiments, the disease or disorder is cancer.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In some embodiments, the alkyl radical is a C₁-C₆ group. In some embodiments, and to the extent not disclosed otherwise for any one or more groups of the compounds of formula (I), the alkyl radical is a C₀-C₆, C₀-C₅, C₀-C₃, C₁-C₆, C₁-C₅, C₁-C₄ or C₁-C₃ group (wherein Co alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. In some embodiments, an alkyl group is a C₁-C₃ alkyl group. In some embodiments, an alkyl group is a C₁-C₂ alkyl group. In some embodiments, an alkyl group is a methyl group.

As used herein, the term “alkylene” refers to a straight or branched divalent hydrocarbon chain linking the rest of the molecule to a radical group, consisting solely of carbon and hydrogen, containing no unsaturation and having from one to six carbon atoms, for example, methylene, ethylene, propylene, n-butylene, and the like. The alkylene chain may be attached to the rest of the molecule through a single bond and to the radical group through a single bond. In some embodiments, and to the extent not disclosed otherwise for any one or more groups of the compounds of formula (I), an alkylene group contains one to four carbon atoms (C₁-C₄ alkylene).

In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (Ci alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, the alkenyl radical is a C₂-C₁₅ group. In some embodiments, and to the extent not disclosed otherwise for any one or more groups of the compounds of formula (I), the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In some embodiments, the alkynyl radical is a C₂-C₁₅ group. In some embodiments, and to the extent not disclosed otherwise for any one or more groups of the compounds of formula (I), the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. In some embodiments, the alkoxyl group is methoxy, ethoxy, propyloxy, or tert-butoxy. An “ether” is two hydrocarbyl groups covalently linked by an oxygen. Accordingly, the substituent of an alkyl that renders that alkyl an ether is or resembles an alkoxyl, such as can be represented by one of —O— alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Therefore, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 12 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 10 carbon atoms (C₃-C₁₀). In one embodiment, carbocyclyl includes 3 to 6 carbon atoms (C₃-C₆). In one embodiment, carbocyclyl includes 5 to 6 carbon atoms (C₅-C₆). In some embodiments, carbocyclyl, as a bicycle, includes C₆-C₁₀. In another embodiment, carbocyclyl, as a spiro system, includes C₅-C₁₁.

Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and phenyl; bicyclic carbocyclyls having 7 to 11 ring atoms include [4,3], [4,4], [4,5], [5,5], [5,6] or [6,6]ring systems, such as for example bicyclo[2.2.1]heptane, bicyclo[2.2.2]octane, naphthalene, and bicyclo[3.2.2]nonane.

Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Therefore, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group),“aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, biphenyl, 1,2,3,4-tetrahydronaphthalenyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Therefore, the term aryl embraces aralkyl groups (e.g., benzyl) which as disclosed above refer to a group of the formula —R^{c}-aryl where R′ is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R′ is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, 4, or 5) carbon atoms have been replaced with a heteroatom or heteroatom-containing group (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3- to 12-membered heterocyclyl ring system. In some embodiments, a heterocyclyl refers to a saturated ring system, such as a 3- to 12-membered saturated heterocyclyl ring system. In some embodiments, a heterocyclyl refers to a heteroaryl ring system, such as a 5- to 12-membered heteroaryl ring system. The term heterocyclyl also includes C₂-C₈ heterocycloalkyl, which is a saturated or partially unsaturated mono-, bi-, or spiro-ring system containing 2-8 carbons and one or more (e.g., 1, 2, or 3) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from O, N, and S. In some embodiments, heterocyclyl includes 4- to 6-membered monocycles having one or more heteroatoms selected from O, N, and S. In some embodiments, heterocyclyl includes 3-membered monocycles. In some embodiments, heterocyclyl includes 4-membered monocycles. In some embodiments, heterocyclyl includes 5- to 6-membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be substituted (e.g., methyl, isopropyl) and/or quaternized (e.g., [NR₄]*Cl⁻, [NR₄]⁺OH⁻).

Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl (e.g., thiazol-2-yl), thiadiazolyl (e.g., 1,3,4-thiadiazol-5-yl and 1,2,4-thiadiazol-5-yl), oxazolyl (e.g., oxazol-2-yl), and oxadiazolyl (e.g., 1,3,4-oxadiazol-5-yl and 1,2,4-oxadiazol-5-yl). Example of 5-membered heterocyclyls containing 2 to 4 nitrogen atoms include imidazolyl (e.g., imidazol-2-yl), triazolyl (e.g., 1,3,4-triazol-5-yl, 1,2,3-triazol-5-yl, and 1,2,4-triazol-5-yl), and tetrazolyl (e.g., 1H-tetrazol-5-yl). Representative examples of benzo-fused 5-membered heterocyclyls include benzoxazol-2-yl, benzthiazol-2-yl and benzimidazol-2-yl. Example of 6-membered heterocyclyls containing one to three nitrogen atoms and optionally a sulfur or oxygen atom are pyridyl (e.g., pyrid-2-yl, pyrid-3-yl, and pyrid-4-yl), pyrimidyl (e.g., pyrimid-2-yl and pyrimid-4-yl), triazinyl (e.g., 1,3,4-triazin-2-yl and 1,3,5-triazin-4-yl), pyridazinyl (e.g., pyridazin-3-yl), and pyrazinyl. In some embodiments, a heterocyclic group includes a heterocyclic ring fused to one or more (e.g., 1 or 2) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Therefore, the term heterocyclic embraces N-heterocyclyl groups which as used herein refer to a heterocyclyl group containing at least one nitrogen atom and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, 1-pyrazolidinyl, 1-imidazolinyl and 1-imidazolidinyl.

The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group.

Representative examples of C-heterocyclyl radicals include 2- or 3-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain.

The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”)) refers to a monocyclic, bicyclic or tricyclic ring system having 5 to 12 ring atoms, wherein at least one ring is aromatic and contains at least one heteroatom. In one embodiment, heteroaryl includes 5- to 6-membered monocyclic aromatic groups where one or more ring atoms is O, N, or S.

Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, and 1,2,3-triazol-5-yl. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1 or 2) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Therefore, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s) in the compounds of formula (I), any of the groups described herein may be substituted or unsubstituted. To the extent not disclosed otherwise for any particular group(s), representative examples of substituents may include alkyl (e.g., C₁-C₆, C₁-C₅, C₁-C₄, C₁-C₃, C₁-C₂, C₁), substituted alkyl (e.g., substituted C₁-C₆, C₁-C₅, C₁-C₄, C₁-C₃, C₁-C₂, Ci), alkoxy (e.g., C₁-C₆, C₁-C₅, C₁-C₄, C₁-C₃, C₁-C₂, Ci), substituted alkoxy (e.g., substituted C₁-C₆, C₁-C₅, C₁-C₄, C₁-C₃, C₁-C₂, Ci), haloalkyl (e.g., CF₃), alkenyl (e.g., C₂-C₆, C₂-C₅, C₂-C₄, C₂-C₃, C₂), substituted alkenyl (e.g., substituted C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., substituted C₂-C₆, C₂-C₅, C₂-C₄, C₂-C₃, C₂), cyclic (e.g., C₃-C₁₂, C₅-C₆), substituted cyclic (e.g., substituted C₃-C₁₂, C₅-C₆), carbocyclic (e.g., C₃-C₁₂, C₅-C₆), substituted carbocyclic (e.g., substituted C₃-C₁₂, C₅-C₆), heterocyclic (e.g., 3- to 12-membered, 5- to 6-membered), substituted heterocyclic (e.g., substituted 3- to 12-membered, 5- to 6-membered), aryl (e.g., benzyl and phenyl), substituted aryl (e.g., substituted benzyl or substituted phenyl), heteroaryl (e.g., pyridyl or pyrimidyl), substituted heteroaryl (e.g., substituted pyridyl or substituted pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., substituted C₆-C₁₂, C₆), alkylthio (e.g., C₁-C₆), substituted alkylthio (e.g., substituted C₁-C₆), arylthio (e.g., C₆-C₁₂, C₆), substituted arylthio (e.g., substituted C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the invention are represented by formula (I):

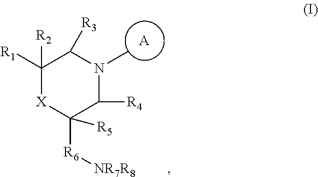

or a pharmaceutically acceptable salt or stereoisomer thereof,
wherein:
X is CH₂, S, CHF, CHCl, CHOH, or CF₂;
R₁ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
each R₉ is independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups;
each R₁₀ is independently alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups, or
R₁ and R₂ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₁₀ groups;
R₃ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₄ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₅ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₆ is absent, (C₁-C₆)alkylene, or (C₃-C₇)carbocyclyl; wherein said alkylene or carbocyclyl, is further optionally substituted by one or more, identical or different R₁₀ groups, or
R₅ and R₆ together with the same carbon atom to which they are attached form a spiro (C₃-C₇)carbocyclyl group or a 4- to 7-membered heterocyclyl group; wherein said carbocyclyl or heterocyclyl, is further optionally substituted by one or more, identical or different R₁₀ groups, or
R₆ is (C₂-C₄)alkylene which is bound to R₇ to form a 4- to 6-membered heterocyclyl group;
R₇ and R₈ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups, or
   R₇ and R₈ together with the nitrogen atom to which they are attached form a 3- to 7-membered heterocyclyl, wherein said heterocyclyl is further optionally substituted by one or more, identical or different R₁₀ groups;

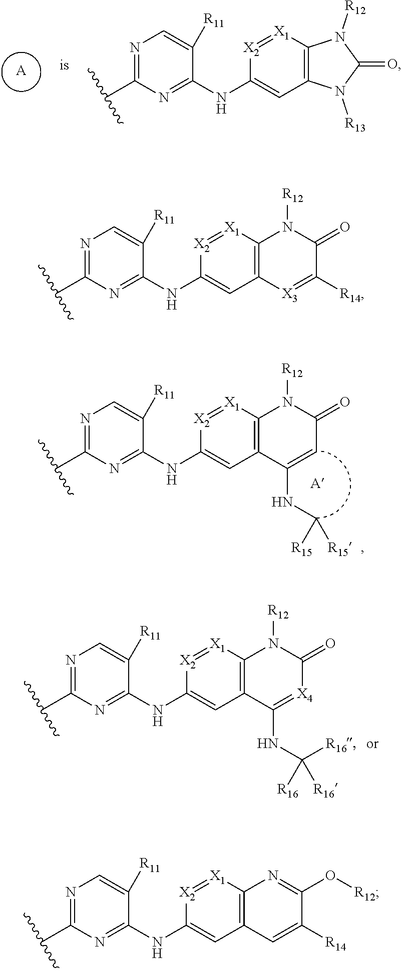

X₁ and X₂ are independently CR₁₇, or N;
   R₁₇ is hydrogen, (C₁-C₄)alkyl, halo, hydroxy, amino, (C₁-C₄)alkoxy, (C₁-C₄)haloalkyl, (C₁-C₄)haloalkoxy, (C₂-C₄)alkenyl, (C₂-C₄)alkynyl, nitro, cyano, NH(C₁-C₄)alkyl, or N(C₁-C₄ alkyl)₂;
X₃ is CH or N;
   X₄ is CR_{17′}, or N; and
      R_{17′} is hydrogen, fluor, chloro, or methyl;
R₁₁ is Cl or CN;
R₁₂ is hydrogen, (C₁-C₆)alkyl, (C₃-C₆)carbocyclyl, 4- to 7-membered heterocyclyl, (C₃-C₇)carbocyclyl(C₁-C₆)alkyl, or 4- to 7-membered heterocyclyl(C₁-C₆)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl, or R₁₂ is -L-Y—Z;
   L is absent or (C₁-C₅)alkylene optionally substituted by one or more substituents selected from (C₁-C₂)alkyl and oxo;
   Y is absent, O, S, S(O), S(O)₂, NR′, C(O), C(O)O, OC(O), C(O)N(R′), N(R′)C(O), N(R′)C(O)N(R′), N(R′)C(O)O, OC(O)N(R′), S(O)₂N(R′), or N(R′)S(O)₂;
      each R′ is independently hydrogen or (C₁-C₄)alkyl;
   Z is hydrogen, (C₁-C₆)alkyl, (C₂-C₆)alkenyl, (C₂-C₆)alkynyl, (C₃-C₁₀)carbocyclyl, or 3- to 10-membered heterocyclyl; wherein Z is optionally substituted by one or more substituents independently selected from (C₁-C₄)alkyl, halo, (C₁-C₄)haloalkyl, (C₁-C₄)haloalkoxy, amino, (C₁-C₄)aminoalkyl, cyano, hydroxy, carboxy, carbamoyl, sulphamoyl, mercapto, ureido, NR^{r}R^{s}, OR^{r}, C(O)R^{r}, C(O)OR^{r}, OC(O)R^{r}, C(O)NR^{r}R^{s}, N(R^{r})C(O)R^{r}, S(O)₀₋₂R^{r}, S(O)₂NR^{r}R^{s}, N(R^{r})SO₂R^{r}, Si(R^{r})(R^{s})R^{t} and (CH₂)₁₋₃NR^{r}R^{s}; wherein R^{r}, R^{s}, and R^{t} are each independently hydrogen, (C₁-C₆)alkyl, or (C₃-C₆)cycloalkyl; or R^{r} and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, and hydroxy;
      R₁₃ is hydrogen, methyl, —(CH₂)₁₋₃W₁W₂, or

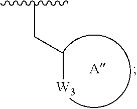

W₁ is CR₁₈R_{18′} or C(O);
   R₁₈ and R_{18′} are independently hydrogen, (C₁-C₂)alkyl, fluoro, hydroxy, cyano, nitro, (C₁-C₂)alkoxy, (C₁-C₂)haloalkyl, (C₁-C₂)haloalkoxy, amino, NH(C₁-C₂)alkyl, or N(C₁-C₂ alkyl)₂, or
R₁₈ and R_{18′} together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
W₂ is cyano, hydroxy, 5- or 6-membered heteroaryl, phenyl, C(O)—(C₁-C₂)alkyl, S(O)₂—(C₁-C₂)alkyl, C(O)OCH₃, C(O)NHCH₃, CR₁₉R₂₀R₂₁, amino, NH(C₁-C₂)alkyl, or N(C₁-C₂ alkyl)₂:
   R₁₉ is hydrogen, (C₁-C₂)alkyl, fluoro, chloro, bromo, hydroxy, amino, cyano, nitro, (C₁-C₂)alkoxy, (C₁-C₂)haloalkyl, or (C₁-C₂)haloalkoxy;
   R₂₀ is hydrogen, (C₁-C₂)alkyl, fluoro, chloro, bromo, hydroxy, cyano, nitro, (C₁-C₂)alkoxy, (C₁-C₂)haloalkyl, (C₁-C₂)haloalkoxy, or —Y₂-L₂-Z₂;
      Y₂ is absent, O, S, S(O), S(O)₂, NR′, C(O), C(O)O, OC(O), C(O)N(R′), N(R′)C(O), S(O)₂N(R′), or N(R′)SO₂;
      L₂ is absent or (C₁-C₂)alkylene;
      Z₂ is hydrogen, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, hydroxy, C(O)R′, C(O)OR′, OC(O)R′, C(O)NR′R′, and N(R′)C(O)R′, wherein each R′ is independently hydrogen or (C₁-C₄)alkyl;
   or R₁₉ and R₂₀ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
R₂₁ is (C₁-C₂)alkyl, —C(O)OR″, OR″, —C(O)NR″, NR″R″, phenyl, or 5-membered heteroaryl, wherein each R″ is independently hydrogen or (C₁-C₂)alkyl;
A″ is (C₄-C₆)carbocyclyl or 4- to 6-membered heterocyclyl, optionally substituted with one or more substituents independently selected from (C₁-C₂)alkyl, halo, hydroxy, cyano, and (C₁-C₂)alkoxy;
W₃ is NR₂₂ or CR₂₃R_{23′};
   R₂₂ is hydrogen, (C₁-C₂)alkyl, (C₁-C₄)haloalkyl, (C₁-C₄)hydroxyalkyl, —C(O)CH₃, or —C(O)O—(C₁-C₄)alkyl;
   R₂₃ and R_{23′} are independently hydrogen, (C₁-C₂)alkyl, cyclopropyl, fluoro, chloro, bromo, hydroxy, amino, cyano, nitro, (C₁-C₂)haloalkyl, (C₁-C₂)haloalkoxy, (C₁-C₂)alkoxy, —C(O)OR″, NR″R″, phenyl, or 5-membered heteroaryl;
      R₁₄ is -L₃CR₂₄R₂₅R₂₆, or —CH═CH—R₂₆;
L₃ is absent, O, S, (C₁-C₄)alkylene, —O—(C₁-C₄)alkylene, or —S—(C₁-C₄)alkylene;
R₂₄ is hydrogen or (C₁-C₄)alkyl;
R₂₅ is hydrogen or (C₁-C₄)alkyl, or
R₂₄ and R₂₅ together with the carbon atom to which they are attached form a (C₃-C₅)carbocyclyl, 4- to 7-membered heterocyclyl, or C═O;
R₂₆ is (C₁-C₆)alkyl, —NR₂₇R₂₈, —OR₂₇, —C(O)R₂₇, —C(O)OR₂₇, —N(R₂₈)C(O)R₂₇, —C(O)NR₂₇R₂₈, —S(O)—(C₁-C₆)alkyl, —S(O)₂—(C₁-C₆)alkyl, —P(O)—(C₁-C₆ alkyl)₂, —C(NH)NH₂, or —(C₁-C₄)alkyl-NR₂₈C(O)R₂₇;
   R₂₇ is hydrogen, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
   R₂₈ is hydrogen or (C₁-C₄)alkyl;
R₁₅ is hydrogen, (C₁-C₄)alkyl, (C₃-C₆)cycloalkyl, (C₁-C₄)haloalkyl, or cyano, wherein said alkyl or cycloalkyl is optionally substituted by one or more substituents selected from (C₁-C₄)alkyl, (C₃-C₆)cycloalkyl, hydroxy, (C₁-C₂)alkoxy, amino, NH(C₁-C₂)alkyl, N((C₁-C₂)alkyl)₂, (C₁-C₂)aminoalkyl, and halo;
R₁₅′ is hydrogen, (C₁-C₄)alkyl, cyano, (C₁-C₄)haloalkyl, or —Y₃-L₄-Z₃;
   Y₃ is absent, C(O)O, or C(O)N(R′);
   L₄ is absent or (C₁-C₂)alkylene;
   Z₃ is hydrogen, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, amino, nitro, cyano, and hydroxy, or
      R¹⁵ and R₁₅′, together with the carbon atom to which they are attached, form a (C₄-C₆)carbocyclyl, or 4- to 6-membered heterocyclyl;
A′ is a 6- or 7-membered heterocyclyl, which in addition to R₁₅ and R₁₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, amino, cyano, and hydroxy;
R₁₆ is hydrogen, (C₁-C₂)alkyl, (C₃-C₄)cycloalkyl, (C₁-C₂)haloalkyl, cyano, (C₂-C₄)alkenyl, or (C₂-C₄)alkynyl;
R₁₆′ is (C₁-C₄)alkyl, cyano, (C₁-C₄)haloalkyl, or —Y₄-L₅-Z₄;
   Y₄ is absent, C(O), C(O)O, OC(O), C(O)N(R′), or S(O)₂N(R′);
   L₅ is absent or (C₁-C₂)alkylene optionally substituted by one or more substituents selected from (C₁-C₂)alkyl and oxo;
   Z₄ is hydrogen, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, amino, nitro, cyano, hydroxy, C(O)R^{u}, C(O)OR^{u}, OC(O)R^{u}, C(O)NR^{u}R^{u}, and N(R^{u})C(O)R^{u}, wherein each R^{u} is independently hydrogen, (C₁-C₄)alkyl, or (C₃-C₆)cycloalkyl, or
Z₄ is -Q-L₆-W, wherein
   Q is absent, O, NH, or N(C₁-C₂)alkyl;
   L₆ is absent or (C₁-C₂)alkylene optionally substituted by one or more substituents selected from oxo and (C₁-C₂)alkyl;
   W is (C₁-C₄)alkyl, phenyl, (C₃-C₆)cycloalkyl, (C₃-C₆)cycloalkenyl, or 5- or 6-membered heterocyclyl, wherein W is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, nitro, cyano, or hydroxy, or
      R₁₆ and R₁₆′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; and
R₁₆″ is hydrogen, (C₁-C₄)alkyl, (C₁-C₂)haloalkyl, (C₁-C₂)alkoxy, (C₁-C₂)haloalkoxy, cyano, nitro, acetylenyl, phenyl, or 5- or 6-membered heteroaryl, wherein said alkyl, phenyl, or heteroaryl is optionally substituted by one or more substituents independently selected from halo, hydroxy, and amino;
   provided that when R₇ is (C₁-C₃)alkyl and R₈ is (C₁-C₃)alkyl, R₆ is not (C₁-C₆)alkylene, and provided that when

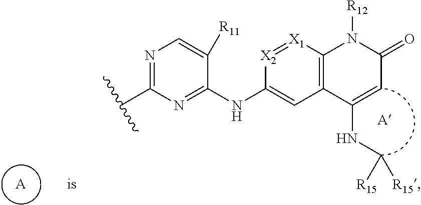

R₁₁ is Cl, X is CH₂ or CF₂, and R₆ is absent or optionally substituted (C₁-C₂)alkylene, R₇ and R₈ cannot be a) independently hydrogen or (C₁-C₂)alkyl optionally substituted with an oxo group, or b) R₇ and R₈ together with the nitrogen atom to which they are attached form a 4-membered heterocyclyl optionally substituted with a gem-difluoro group.

In some embodiments, L and Y are absent and Z is methyl.

In some embodiments, R₁₁ is Cl.

In some embodiments, R₁₁ is CN.

In some embodiments, R₁ is hydrogen, —OR₉, or (C₁-C₆)alkyl.

In some embodiments, R₁ is hydrogen, methyl, or —OH.

In some embodiments, R₁ is hydrogen.

In some embodiments, R₁ is methyl.

In some embodiments, R₂ is hydrogen, —OR₉, or (C₁-C₆)alkyl.

In some embodiments, R₂ is hydrogen, methyl, or —OH.

In some embodiments, R₂ is hydrogen.

In some embodiments, R₂ is methyl.

In some embodiments, R₁ and R₂ together with the same carbon atom to which they are attached form a spiro (C₃-C₇)carbocyclyl group.

In some embodiments, R₁ and R₂ together with the same carbon atom to which they are attached form a spiro (C₃)carbocyclyl.

In some embodiments, R₃ is hydrogen.

In some embodiments, R₄ is hydrogen.

In some embodiments, R₅ is hydrogen, —OR₉, or (C₁-C₆)alkyl.

In some embodiments, R₅ is hydrogen, methyl, or —OH.

In some embodiments, R₅ is hydrogen.

In some embodiments, R₅ is methyl.

In some embodiments, R₆ is absent.

In some embodiments, R₆ is (C₁-C₆)alkyl or (C₃-C₇)carbocyclyl.

In some embodiments, R₆ is (C₁-C₂)alkyl or (C₃-C₄)carbocyclyl.

In some embodiments, R₆ is C₁-alkylene. In some embodiments, R₆ is C₂-alkylene. In some embodiments, R₆ is C₁-alkylene substituted by one or more, identical or different R₁₀ groups.

In some embodiments, R₆ is C₂-alkylene substituted by one or more, identical or different Rio groups. In some embodiments, each R₁₀ is independently methyl, fluoro, or cyclopropyl.

In some embodiments, R₆ is C₃-carbocyclyl. In some embodiments, R₆ is C₄-carbocyclyl.

In some embodiments, R₅ and R₆ together with the same carbon atom to which they are attached form a spiro (C₃-C₇)carbocyclyl group.

In some embodiments, R₅ and R₆ together with the same carbon atom to which they are attached form a spiro (C₃)carbocyclyl.

In some embodiments, R₆ is (C₂-C₄)alkylene which is bound to R₇ to form a 4- to 6-membered heterocyclyl group.

In some embodiments, R₆ is (C₂-C₃)alkylene which is bound to R₇ to form a 4-membered heterocyclyl group.

In some embodiments, R₇ is hydrogen or (C₁-C₆)alkyl.

In some embodiments, R₇ is hydrogen or methyl.

In some embodiments, R₅ is hydrogen or (C₁-C₆)alkyl.

In some embodiments, R₅ is hydrogen or methyl.

In some embodiments, R₇ and R₈ together with the nitrogen atom to which they are attached form a 3- to 7-membered heterocyclyl.

In some embodiments, R₇ and R₈ together with the nitrogen atom to which they are attached form a 4-membered heterocyclyl.

In some embodiments, X is CH₂.

In some embodiments, X is S.

In some embodiments, X is CHF.

In some embodiments, X is CHCL.

In some embodiments, X is CHOH.

In some embodiments, X is CF₂.

In some embodiments,

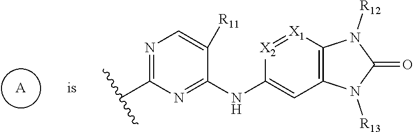

and the compound of formula (I) has the structure of formula I-1,

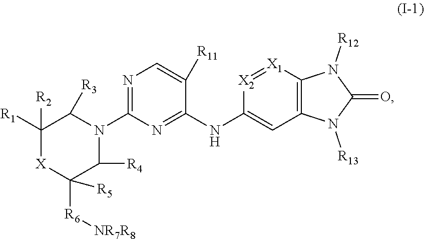

or a pharmaceutically acceptable salt or stereoisomer thereof.

In some embodiments of formula I-1, X₁ is N. In some embodiments of formula I-1, X₁ is CR₁₇. In some embodiments of formula I-1, X₁ is CH.

In some embodiments of formula I-1, X₂ is N. In some embodiments of formula I-1, X₂ is CR₁₇. In some embodiments of formula I-1, X₂ is CH.

In some embodiments of formula I-1, X₁ and X₂ are both CH.

In some embodiments of formula I-1, X is CH₂.

In some embodiments of formula I-1, X is CHF.

In some embodiments of formula I-1, X is CF₂.

In some embodiments of formula I-1, R₁ is hydrogen, —OR₉, or (C₁-C₆)alkyl.

In some embodiments of formula I-1, R₁ is hydrogen, methyl, or —OH.

In some embodiments of formula I-1, R₁ is hydrogen.

In some embodiments of formula I-1, Riis methyl.

In some embodiments of formula I-1, R₂ is hydrogen, —OR₉, or (C₁-C₆)alkyl.

In some embodiments of formula I-1, R₂ is hydrogen, methyl, or —OH.

In some embodiments of formula I-1, R₂ is hydrogen.

In some embodiments of formula I-1, R₂ is methyl.

In some embodiments of formula I-1, R₁ and R₂ together with the same carbon atom to which they are attached form a spiro (C₃-C₇)carbocyclyl group.

In some embodiments of formula I-1, R₁ and R₂ together with the same carbon atom to which they are attached form a spiro (C₃)carbocyclyl.

In some embodiments of formula I-1, R₃ is hydrogen.

In some embodiments of formula I-1, R₄ is hydrogen.

In some embodiments of formula I-1, R₅ is hydrogen, —OR₉, or (C₁-C₆)alkyl.

In some embodiments of formula I-1, R₅ is hydrogen, methyl, or —OH.

In some embodiments of formula I-1, R₅ is hydrogen.

In some embodiments of formula I-1, R₅ is methyl.

In some embodiments of formula I-1, R₆ is (C₁-C₆)alkyl or (C₃-C₇)carbocyclyl.

In some embodiments of formula I-1, R₆ is (C₁-C₂)alkyl or (C₃-C₄)carbocyclyl.

In some embodiments of formula I-1, R₆ is Cl-alkylene. In some embodiments of formula I-1, R₆ is C₂-alkylene. In some embodiments of formula I-1, R₆ is Cl-alkylene substituted by one or more, identical or different R₁₀ groups. In some embodiments of formula I-1, R₆ is C₂-alkylene substituted by one or more, identical or different R₁₀ groups. In some embodiments of formula I-1, each R₁₀ is independently methyl, fluoro, or cyclopropyl.

In some embodiments of formula I-1, R₆ is C₃-carbocyclyl. In some embodiments of formula I-1, R₆ is C₄-carbocyclyl.

In some embodiments of formula I-1, R₅ and R₆ together with the same carbon atom to which they are attached form a spiro (C₃-C₇)carbocyclyl group.

In some embodiments of formula I-1, R₅ and R₆ together with the same carbon atom to which they are attached form a spiro (C₃)carbocyclyl.

In some embodiments of formula I-1, R₆ is (C₂-C₄)alkylene which is bound to R₇ to form a 4- to 6-membered heterocyclyl group.

In some embodiments of formula I-1, R₆ is (C₂-C₃)alkylene which is bound to R₇ to form a 4-membered heterocyclyl group.

In some embodiments of formula I-1, R₇ is hydrogen or (C₁-C₆)alkyl.

In some embodiments of formula I-1, R₇ is hydrogen or methyl.

In some embodiments of formula I-1, R₅ is hydrogen or (C₁-C₆)alkyl.

In some embodiments of formula I-1, R₅ is hydrogen or methyl.

In some embodiments of formula I-1, R₇ and R₈ together with the nitrogen atom to which they are attached form a 3- to 7-membered heterocyclyl.

In some embodiments of formula I-1, R₇ and R₈ together with the nitrogen atom to which they are attached form a 3-membered heterocyclyl.

In some embodiments of formula I-1, R₁₂ is methyl and R₁₁ is Cl. In some embodiments, R₁₂ is methyl and R₁₁ is CN.

In some embodiments of formula I-1, R₁₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl. In some embodiments, R₁₂ is methyl. In some embodiments, R₁₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-1, R₁₃ is —(CH₂)₁₋₃W₁W₂. In some embodiments of formula I-1, R₁₃ is —(CH₂)₂W₁W₂.

In some embodiments of formula I-1, W₁ is CR₁₈R_{18′}. In some embodiments of formula I-1, R₁₈ and R_{18′} are independently hydrogen or (C₁-C₂)alkyl. In some embodiments of formula I-1, R₁₈ and R_{18′} are both (C₁-C₂)alkyl. In some embodiments of formula I-1, R₁₈ and R_{18′} are both methyl.

In some embodiments of formula I-1, W₂ is cyano, hydroxy, or amino. In some embodiments of formula I-1, W₂ is hydroxy.

In some embodiments, the compound of formula I-1 is of formula I-1a, I-1b, I-1c, I-1d, I-1e, or I-1f:

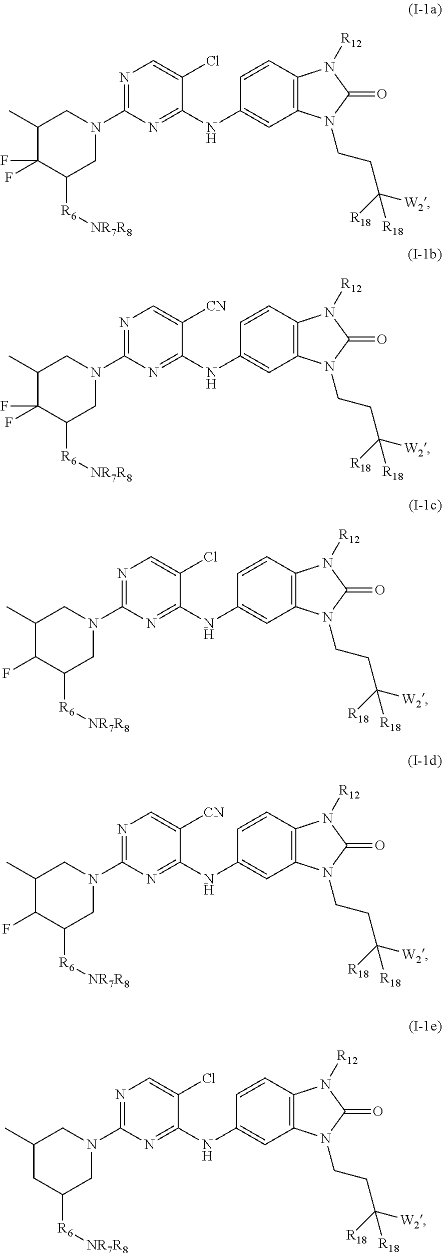

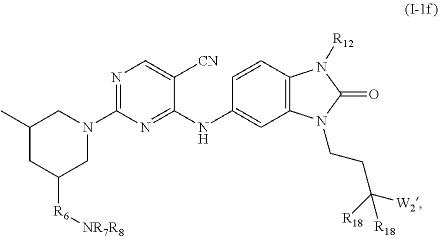

or a pharmaceutically acceptable salt or stereoisomer thereof.

In some embodiments, the compound of formula I-1 is of formula I-1g, I-1h, I-1i, I-1j, I-1k, or I-1l:

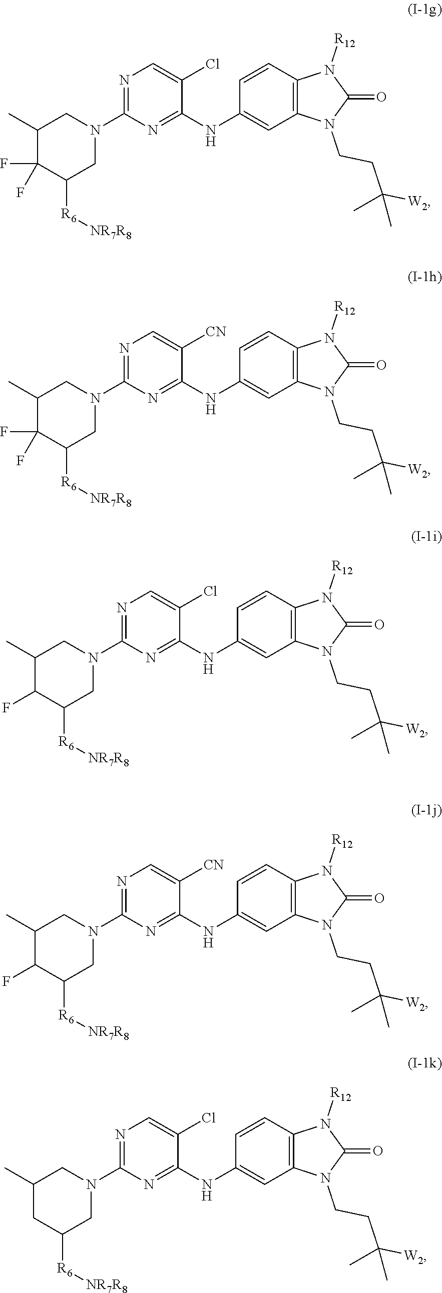

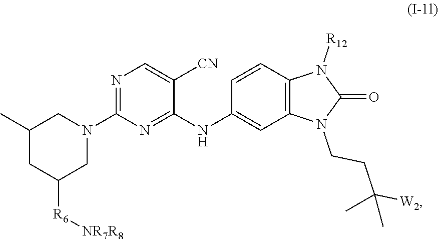

or a pharmaceutically acceptable salt or stereoisomer thereof.

In some embodiments, the compound of formula I-1 is of formula I-1m or I-1n:

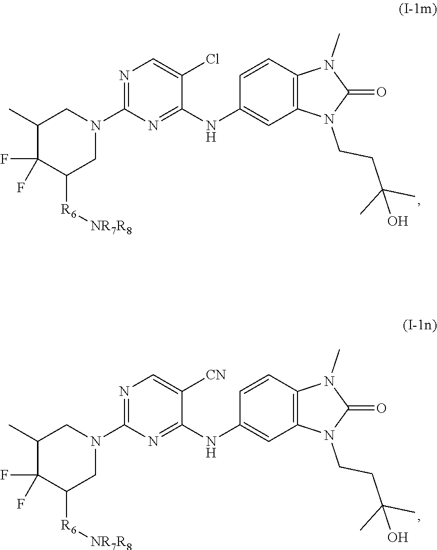

or a pharmaceutically acceptable salt or stereoisomer thereof.

In some embodiments,

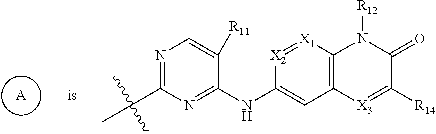

and the compound of formula (I) has the structure of formula I-2,

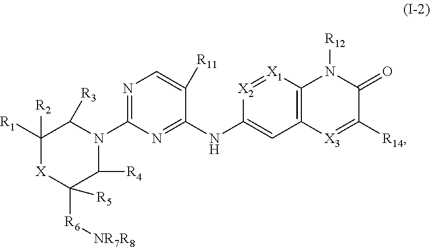

or a pharmaceutically acceptable salt or stereoisomer thereof.

In some embodiments of formula I-2, X₁ is N. In some embodiments of formula I-2, X₁ is CR₁₇. In some embodiments of formula I-2, X₁ is CH.

In some embodiments of formula I-2, X₂ is N. In some embodiments of formula I-2, X₂ is CR₁₇. In some embodiments of formula I-2, X₂ is CH.

In some embodiments of formula I-2, X₁ and X₂ are both CH.

In some embodiments of formula I-2, X₃ is N. In some embodiments of formula I-2, X₃ is CH.

In some embodiments of formula I-2, X is CH₂.

In some embodiments of formula I-2, X is CHF.

In some embodiments of formula I-2, X is CF₂.

In some embodiments of formula I-2, R₁ is hydrogen, —OR₉, or (C₁-C₆)alkyl.

In some embodiments of formula I-2, R₁ is hydrogen, methyl, or —OH.

In some embodiments of formula I-2, R₁ is hydrogen.

In some embodiments of formula I-2, Ri is methyl.

In some embodiments of formula I-2, R₂ is hydrogen, —OR₉, or (C₁-C₆)alkyl.

In some embodiments of formula I-2, R₂ is hydrogen, methyl, or —OH.

In some embodiments of formula I-2, R₂ is hydrogen.

In some embodiments of formula I-2, R₂ is methyl.

In some embodiments of formula I-2, R₁ and R₂ together with the same carbon atom to which they are attached form a spiro (C₃-C₇)carbocyclyl group.

In some embodiments of formula I-2, R₁ and R₂ together with the same carbon atom to which they are attached form a spiro (C₃)carbocyclyl.

In some embodiments of formula I-2, R₃ is hydrogen.

In some embodiments of formula I-2, R₄ is hydrogen.

In some embodiments of formula I-2, R₅ is hydrogen, —OR₉, or (C₁-C₆)alkyl.

In some embodiments of formula I-2, R₅ is hydrogen, methyl, or —OH.

In some embodiments of formula I-2, R₅ is hydrogen.

In some embodiments of formula I-2, R₅ is methyl.

In some embodiments of formula I-2, R₆ is (C₁-C₆)alkyl or (C₃-C₇)carbocyclyl.

In some embodiments of formula I-2, R₆ is (C₁-C₂)alkyl or (C₃-C₄)carbocyclyl.

In some embodiments of formula I-2, R₆ is Cl-alkylene. In some embodiments of formula I-2, R₆ is C₂-alkylene. In some embodiments of formula I-2, R₆ is Cl-alkylene substituted by one or more, identical or different R₁₀ groups. In some embodiments of formula I-2, R₆ is C₂-alkylene substituted by one or more, identical or different R₁₀ groups. In some embodiments of formula I-2, each R₁₀ is independently methyl, fluoro, or cyclopropyl.

In some embodiments of formula I-2, R₆ is C₃-carbocyclyl. In some embodiments of formula I-2, R₆ is C₄-carbocyclyl.

In some embodiments of formula I-2, R₅ and R₆ together with the same carbon atom to which they are attached form a spiro (C₃-C₇)carbocyclyl group.

In some embodiments of formula I-2, R₅ and R₆ together with the same carbon atom to which they are attached form a spiro (C₃)carbocyclyl.

In some embodiments of formula I-2, R₆ is (C₂-C₄)alkylene which is bound to R₇ to form a 4- to 6-membered heterocyclyl group.

In some embodiments of formula I-2, R₆ is (C₂-C₃)alkylene which is bound to R₇ to form a 4-membered heterocyclyl group.

In some embodiments of formula I-2, R₇ is hydrogen or (C₁-C₆)alkyl.

In some embodiments of formula I-2, R₇ is hydrogen or methyl.

In some embodiments of formula I-2, R₅ is hydrogen or (C₁-C₆)alkyl.

In some embodiments of formula I-2, R₈ is hydrogen or methyl.

In some embodiments of formula I-2, R₇ and R₈ together with the nitrogen atom to which they are attached form a 3- to 7-membered heterocyclyl.

In some embodiments of formula I-2, R₇ and R₈ together with the nitrogen atom to which they are attached form a 3-membered heterocyclyl.

In some embodiments of formula I-2, R₁₂ is methyl and R₁₁ is Cl. In some embodiments, R₁₂ is methyl and Ri is CN.

In some embodiments of formula I-2, R₁₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl. In some embodiments, R₁₂ is methyl. In some embodiments, R₁₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-1, R₁₄ is -L₃CR₂₄R₂₅R₂₆. In some embodiments of formula I-2, L₃ is (C₁-C₄)alkylene, —O—(C₁-C₄)alkylene, or —S—(C₁-C₄)alkylene. In some embodiments of formula I-2, L₃ is —O—(C₁-C₄)alkylene. In some embodiments of formula I-2, L₃ is —O—(C₁)alkylene.

In some embodiments of formula I-2, R₂₄ and R₂₅ together with the carbon atom to which they are attached form a (C₃-C₅)carbocyclyl, 4- to 7-membered heterocyclyl, or C═O. In some embodiments of formula I-2, R₂₄ and R₂₅ together with the same carbon atom to which they are attached form C═O. In some embodiments of formula I-2, R₂₄ and R₂₅ together with the same carbon atom to which they are attached form 4- to 7-membered heterocyclyl. In some embodiments of formula I-2, R₂₄ and R₂₅ together with the same carbon atom to which they are attached form an oxetane ring.

In some embodiments of formula I-2, R₂₆ is (C₁-C₆)alkyl, —NR₂₇R₂₈, or —OR₂₇. In some embodiments of formula I-2, R₂₆ is methyl, hydroxyl, amino, or NHMe. In some embodiments, R₂₆ is methyl. In some embodiments, R₂₆ is hydroxyl. In some embodiments, R₂₆ is amino. In some embodiments, R₂₆ is NHMe.

In some embodiments, the compound of formula I-2 is of formula I-2a, I-2b, I-2c, I-2d, I-2e, or I-2f:

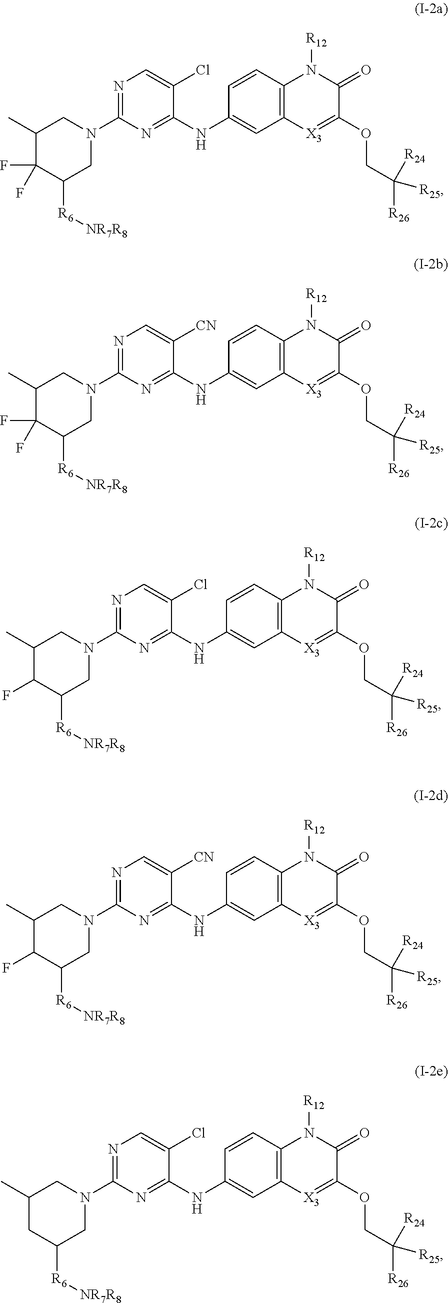

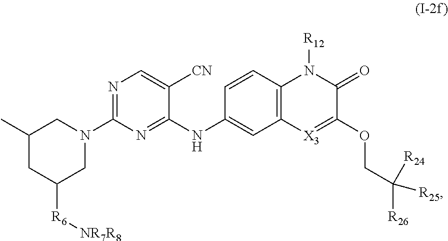

or a pharmaceutically acceptable salt or stereoisomer thereof.

In some embodiments, the compound of formula I-2 is of formula I-2g, I-2h, I-2i, I-2j, I-2k, or I-2l:

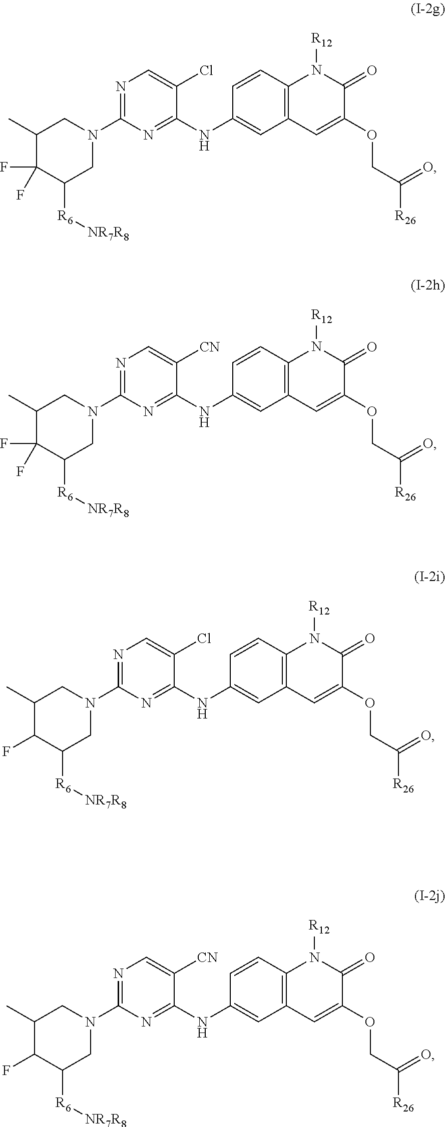

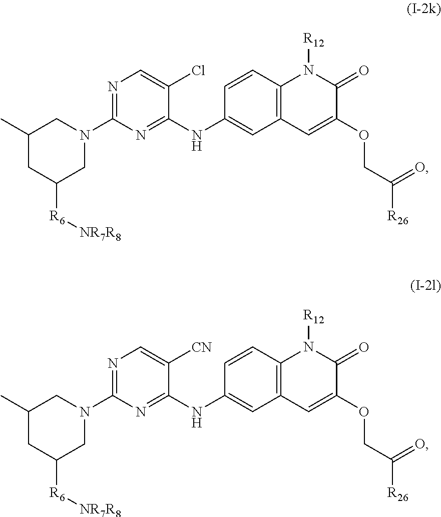

or a pharmaceutically acceptable salt or stereoisomer thereof.

In some embodiments, the compound of formula I-2 is of formula I-2m or I-2n:

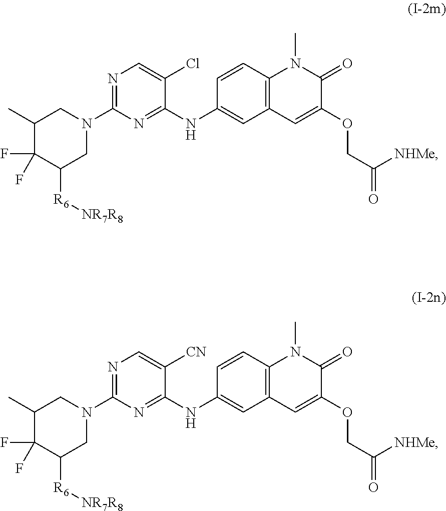

or a pharmaceutically acceptable salt or stereoisomer thereof.

In some embodiments,

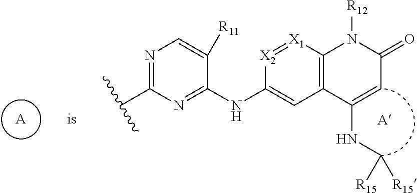

and the compound of formula (I) has the structure of formula I-3,

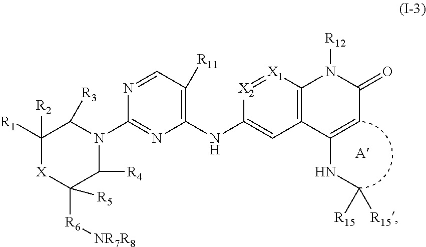

or a pharmaceutically acceptable salt or stereoisomer thereof. In some embodiments, Ri is Cl. In some embodiments, R₁₁ is CN.

In some embodiments of formula I-3, X₁ is CR₁₇. In some embodiments of formula I-3, X₁ is CH. In some embodiments of formula I-3, X₁ is N.

In some embodiments of formula I-3, X₂ is CR₁₇. In some embodiments of formula I-3, X₂ is CH. In some embodiments of formula I-3, X₂ is N.

In some embodiments of formula I-3, R₁₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl. In some embodiments, R₁₂ is methyl. In some embodiments, R₁₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-3, A′ is a 7-membered heterocyclyl, wherein the heterocyclyl contains 2 heteroatoms selected from N and O, and which in addition to R₁₅ and R₁₅′, is optionally further substituted by one more substituents independently selected from oxo, (C₁-C₂)alkyl, cyclopropyl, spiro-cyclopropyl, halo, (C₁-C₂)haloalkyl, (C₁-C₂)alkoxy, amino, cyano, and hydroxy.

In some embodiments, the compound of formula I-3 is of formula I-3a, I-3b, I-3c, I-3d, I-3e, I-3f, I-3g, I-3h, I-3i, I-3j, 1-3k, or I-31:

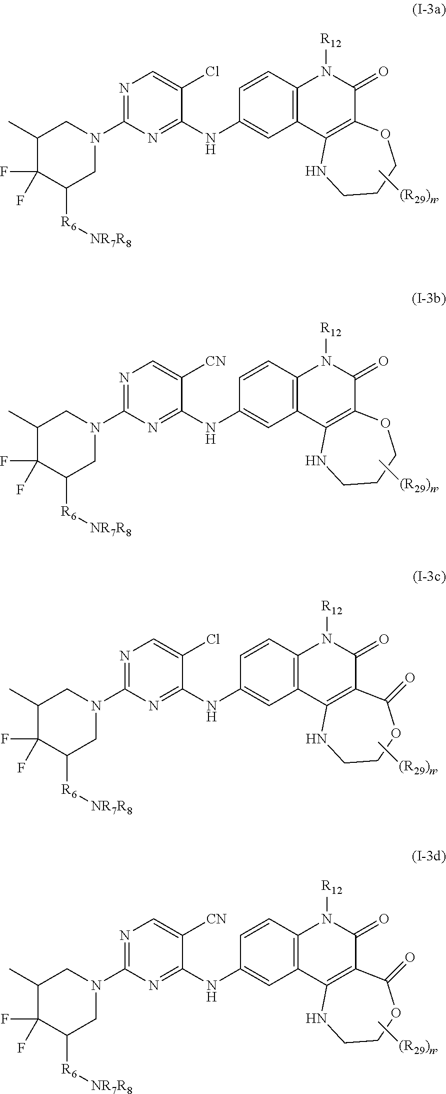

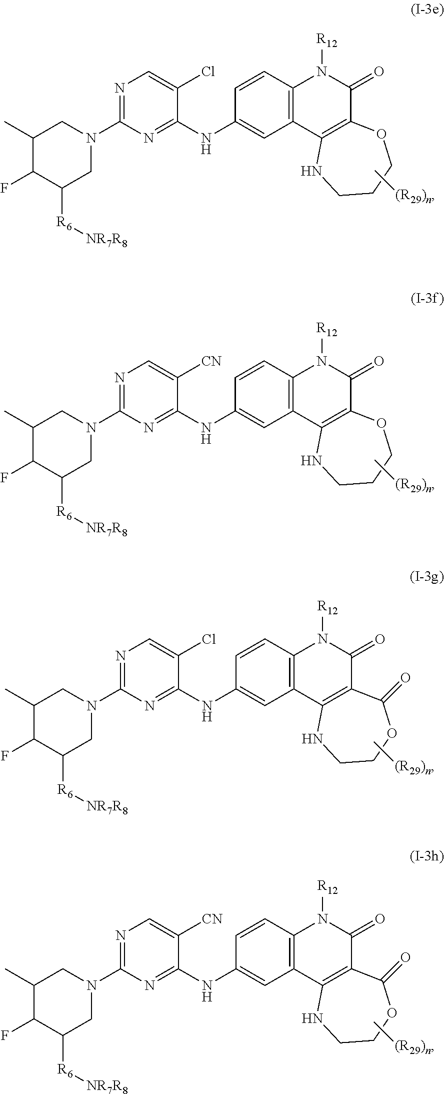

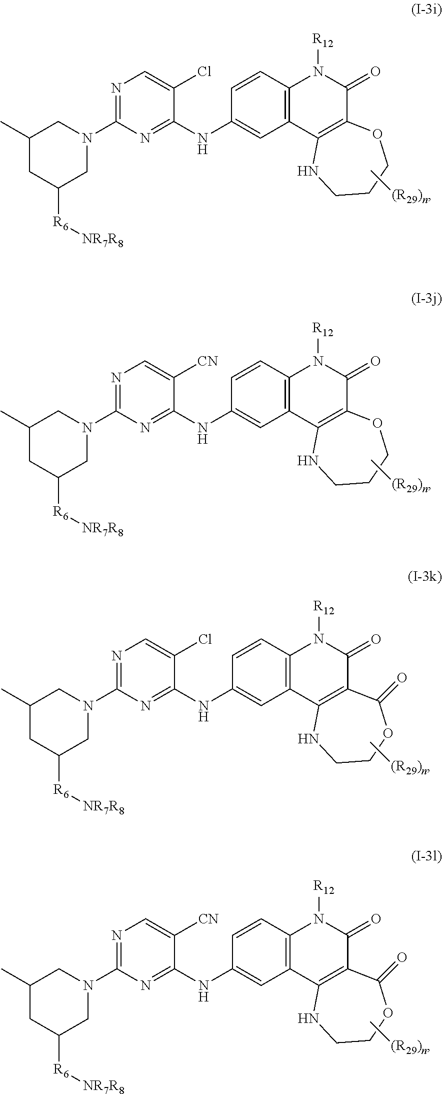

or a pharmaceutically acceptable salt or stereoisomer thereof, wherein each R₂₉ is independently oxo, (C₁-C₂)alkyl, cyclopropyl, spiro-cyclopropyl, halo, (C₁-C₂)haloalkyl, (C₁-C₂)alkoxy, amino, cyano, and hydroxy; and n is 0-2.

In some embodiments, the compound of formula I-3 is of formula I-3m or I-3n:

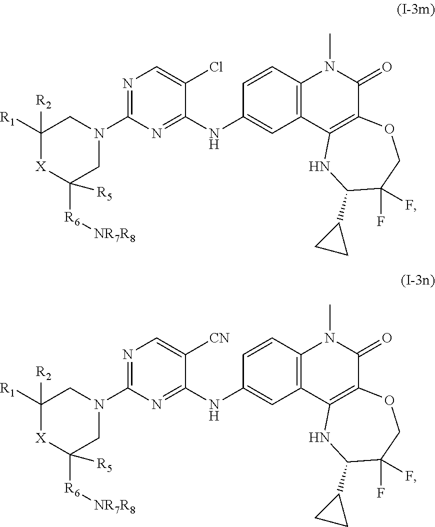

or a pharmaceutically acceptable salt or stereoisomer thereof.

In some embodiments,

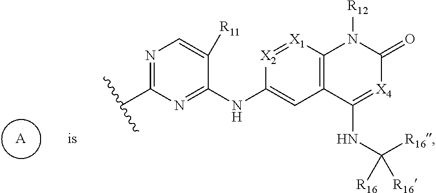

and the compound of formula (I) has the structure of formula I-4,

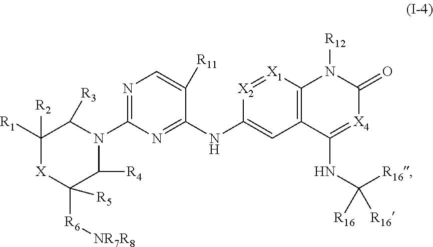

or a pharmaceutically acceptable salt or stereoisomer thereof.

In some embodiments of formula I-4, X₁ is CR₁₇. In some embodiments of formula I-4, X₁ is CH. In some embodiments of formula I-4, X₁ is N.

In some embodiments of formula I-4, X₂ is CR₁₇. In some embodiments of formula I-4, X₂ is CH. In some embodiments of formula I-4, X₂ is N.

In some embodiments of formula I-4, X₄ is CH. In some embodiments of formula I-4, X₄ is N.

In some embodiments of formula I-4, R₁₂ is (C₁-C₂)alkyl, 4-membered heterocyclyl, (C₃)carbocyclyl(C₁)alkyl, or 4-membered heterocyclyl(C₁)alkyl, wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl. In some embodiments, R₁₂ is methyl. In some embodiments, R₁₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments, the compound of formula I-4 is of formula I-4a or I-4b:

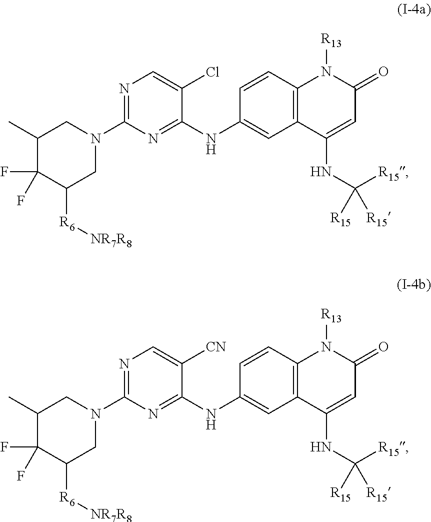

or a pharmaceutically acceptable salt or stereoisomer thereof.

In some embodiments,

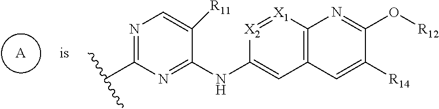

and the compound of formula (I) has the structure of formula I-5,

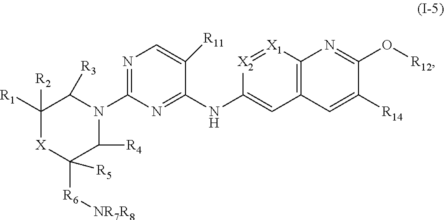

or a pharmaceutically acceptable salt or stereoisomer thereof.

In some embodiments of formula I-5, X₁ is CR₁₇. In some embodiments of formula I-5, X₁ is CH. In some embodiments of formula I-5, X₁ is N.

In some embodiments of formula I-5, X₂ is CR₁₇. In some embodiments of formula I-5, X₂ is CH. In some embodiments of formula I-5, X₂ is N.

In some embodiments of formula I-5, R₁₂ is (C₁-C₂)alkyl, 4-membered heterocyclyl, (C₃)carbocyclyl(C₁)alkyl, or 4-membered heterocyclyl(C₁)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl. In some embodiments, R₁₂ is methyl. In some embodiments, R₁₂ is 4-membered heterocyclyl, (C₃)carbocyclyl(C₁)alkyl, or 4-membered heterocyclyl(C₁)alkyl, wherein the heterocyclyl contains 1 heteroatom selected from N and O.

In some embodiments of formula I-5, R₁₂ is (C₁-C₆)alkyl, (C₁-C₆)alkyl-OH, (C₁-C₆)alkyl-NH(C₁-C₆)alkyl, or (C₁-C₆)alkyl-N((C₁-C₆)alkyl)₂.

In some embodiments of formula I-5, R₁₄ is -L₃CR₂₄R₂₅R₂₆. In some embodiments of formula I-5, L₃ is (C₁-C₄)alkylene, —O—(C₁-C₄)alkylene, or —S—(C₁-C₄)alkylene. In some embodiments of formula I-5, L₃ is —O—(C₁-C₄)alkylene. In some embodiments of formula I-5, L₃ is —O—(C₁)alkylene.

In some embodiments of formula I-5, R₂₄ and R₂₅ together with the carbon atom to which they are attached form a (C₃-C₅)carbocyclyl, 4- to 7-membered heterocyclyl, or C═O. In some embodiments of formula I-5, R₂₄ and R₂₅ together with the same carbon atom to which they are attached form C═O. In some embodiments of formula I-5, R₂₄ and R₂₅ together with the same carbon atom to which they are attached form 4- to 7-membered heterocyclyl. In some embodiments of formula I-5, R₂₄ and R₂₅ together with the same carbon atom to which they are attached form an oxetane ring.

In some embodiments of formula I-5, R₂₆ is (C₁-C₆)alkyl, —NR₂₇R₂₈, or —OR₂₇. In some embodiments of formula I-5, R₂₆ is methyl, hydroxyl, amino, or NHMe. In some embodiments, R₂₆ is methyl. In some embodiments, R₂₆ is hydroxyl. In some embodiments, R₂₆ is amino. In some embodiments, R₂₆ is NHMe.

In some embodiments, the compound of formula I-5 is of formula I-5a, I-5b, I-5c, I-5d, I-5e, or I-5f:

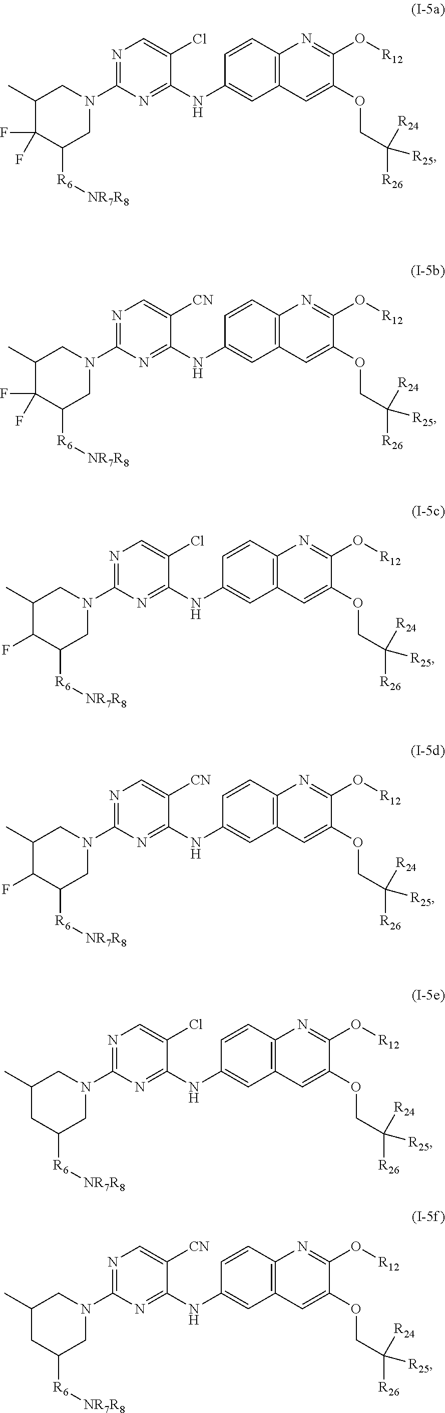

or a pharmaceutically acceptable salt or stereoisomer thereof.

Representative examples of compounds of the invention include:

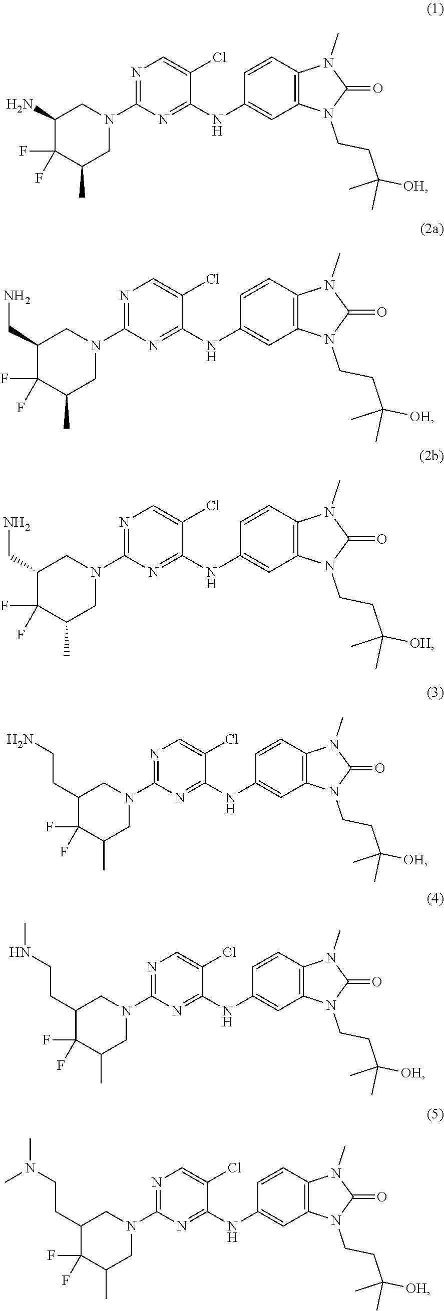 [SMILES: C[C@@H]1CN(c2ncc(Cl)c(Nc3ccc4c(c3)n(CCC(C)(C)O)c(=O)n4C)n2)C[C@H](N)C1(F)F ; C[C@@H]1CN(c2ncc(Cl)c(Nc3ccc4c(c3)n(CCC(C)(C)O)c(=O)n4C)n2)C[C@H](CN)C1(F)F ; C[C@H]1CN(c2ncc(Cl)c(Nc3ccc4c(c3)n(CCC(C)(C)O)c(=O)n4C)n2)C[C@@H](CN)C1(F)F ; CC1CN(c2ncc(Cl)c(Nc3ccc4c(c3)n(CCC(C)(C)O)c(=O)n4C)n2)CC(CCN)C1(F)F ; CNCCC1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(CCN(C)C)C1(F)F]

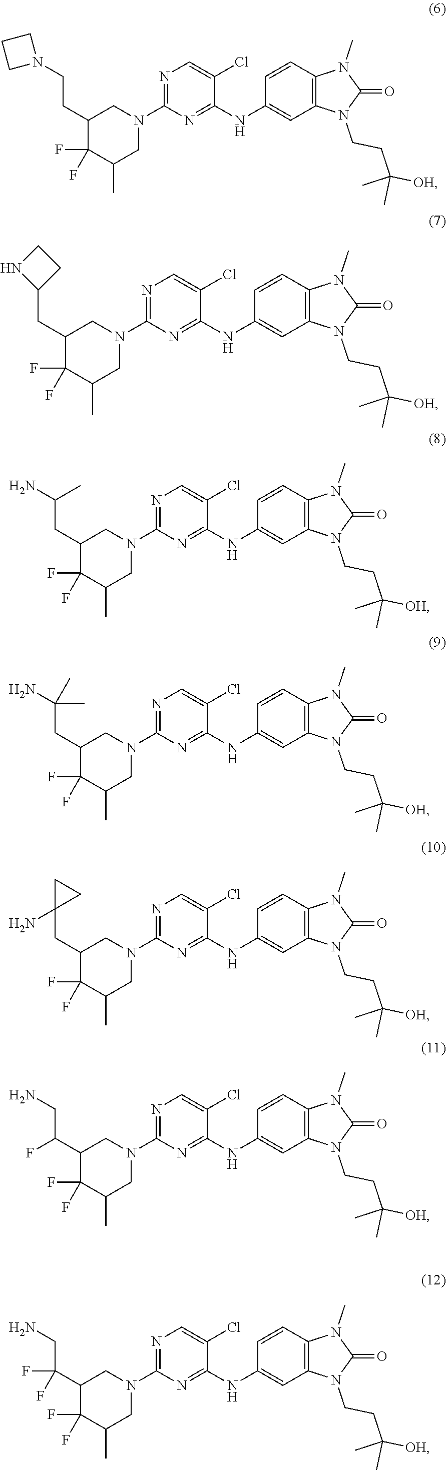

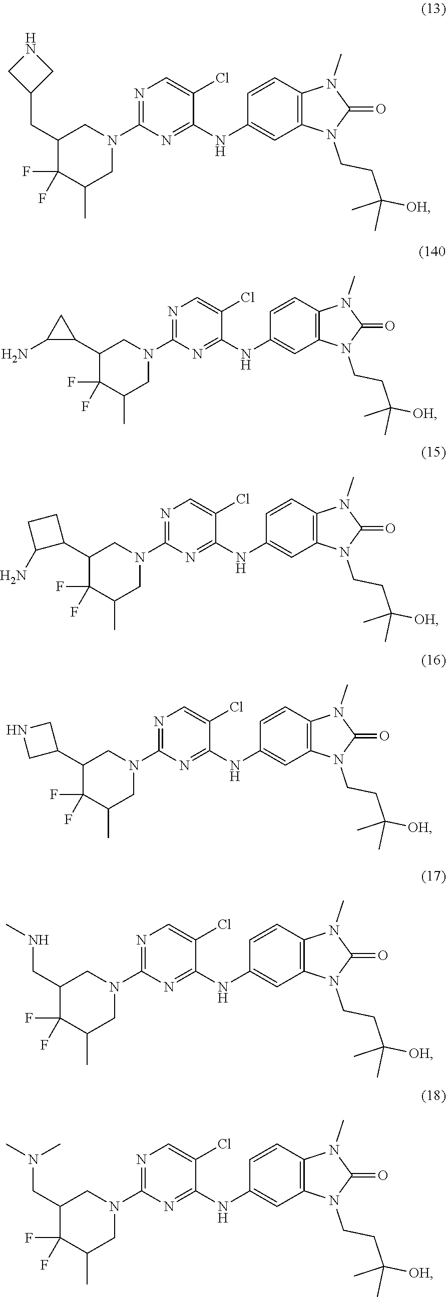 [SMILES: CC1CN(c2ncc(Cl)c(Nc3ccc4c(c3)n(CCC(C)(C)O)c(=O)n4C)n2)CC(CC2CNC2)C1(F)F ; CC1CN(c2ncc(Cl)c(Nc3ccc4c(c3)n(CCC(C)(C)O)c(=O)n4C)n2)CC(C2CC2N)C1(F)F ; CC1CN(c2ncc(Cl)c(Nc3ccc4c(c3)n(CCC(C)(C)O)c(=O)n4C)n2)CC(C2CCC2N)C1(F)F ; CC1CN(c2ncc(Cl)c(Nc3ccc4c(c3)n(CCC(C)(C)O)c(=O)n4C)n2)CC(C2CNC2)C1(F)F ; CNCC1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(CN(C)C)C1(F)F]

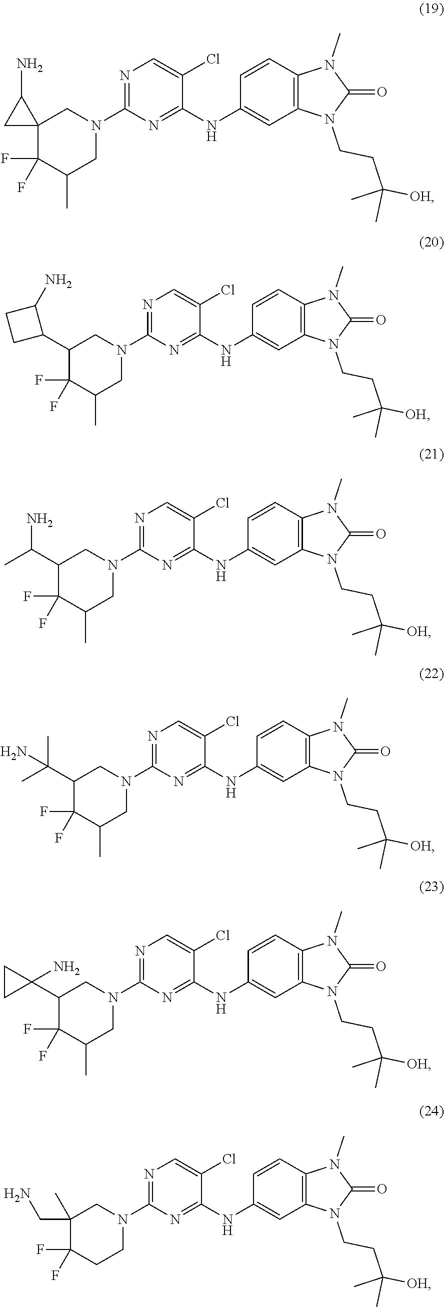 [SMILES: CC1CN(c2ncc(Cl)c(Nc3ccc4c(c3)n(CCC(C)(C)O)c(=O)n4C)n2)CC2(CC2N)C1(F)F ; CC1CN(c2ncc(Cl)c(Nc3ccc4c(c3)n(CCC(C)(C)O)c(=O)n4C)n2)CC(C2CCC2N)C1(F)F ; CC(N)C1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(C(C)(C)N)C1(F)F ; CC1CN(c2ncc(Cl)c(Nc3ccc4c(c3)n(CCC(C)(C)O)c(=O)n4C)n2)CC(C2(N)CC2)C1(F)F ; Cn1c(=O)n(CCC(C)(C)O)c2cc(Nc3nc(N4CCC(F)(F)C(C)(CN)C4)ncc3Cl)ccc21]

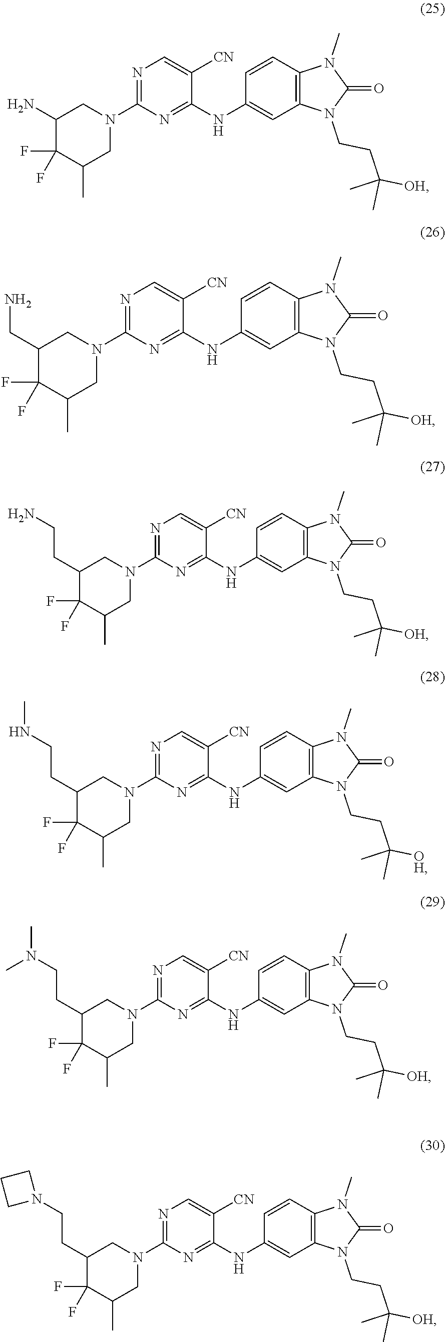 [SMILES: CC1CN(c2ncc(C#N)c(Nc3ccc4c(c3)n(CCC(C)(C)O)c(=O)n4C)n2)CC(N)C1(F)F ; CC1CN(c2ncc(C#N)c(Nc3ccc4c(c3)n(CCC(C)(C)O)c(=O)n4C)n2)CC(CN)C1(F)F ; CC1CN(c2ncc(C#N)c(Nc3ccc4c(c3)n(CCC(C)(C)O)c(=O)n4C)n2)CC(CCN)C1(F)F ; CNCCC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CCN(C)C)C1(F)F ; CC1CN(c2ncc(C#N)c(Nc3ccc4c(c3)n(CCC(C)(C)O)c(=O)n4C)n2)CC(CCN2CCC2)C1(F)F]

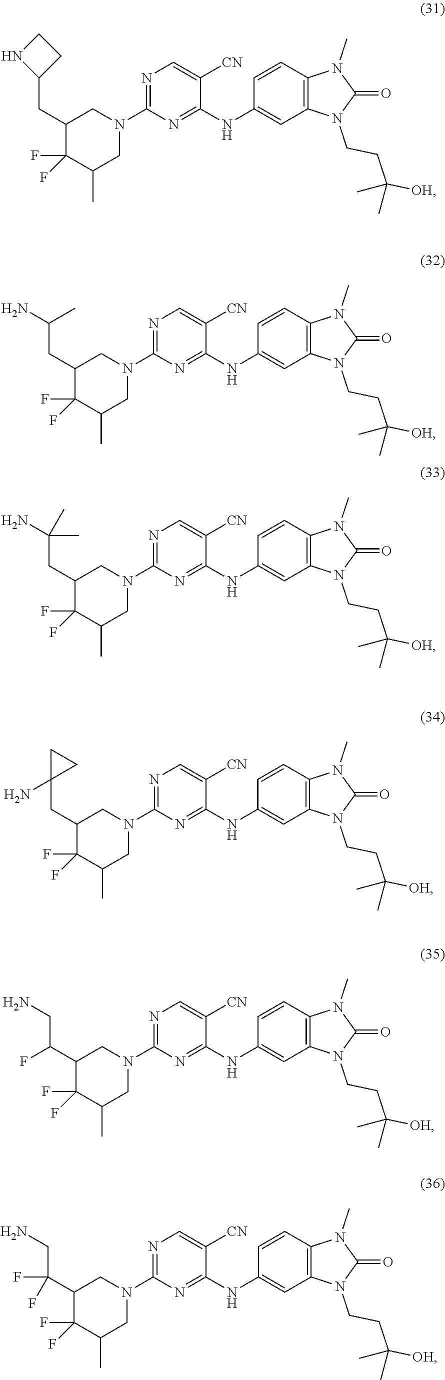 [SMILES: CC1CN(c2ncc(C#N)c(Nc3ccc4c(c3)n(CCC(C)(C)O)c(=O)n4C)n2)CC(CC2CCN2)C1(F)F ; CC(N)CC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CC(C)(C)N)C1(F)F ; CC1CN(c2ncc(C#N)c(Nc3ccc4c(c3)n(CCC(C)(C)O)c(=O)n4C)n2)CC(CC2(N)CC2)C1(F)F ; CC1CN(c2ncc(C#N)c(Nc3ccc4c(c3)n(CCC(C)(C)O)c(=O)n4C)n2)CC(C(F)CN)C1(F)F ; CC1CN(c2ncc(C#N)c(Nc3ccc4c(c3)n(CCC(C)(C)O)c(=O)n4C)n2)CC(C(F)(F)CN)C1(F)F]

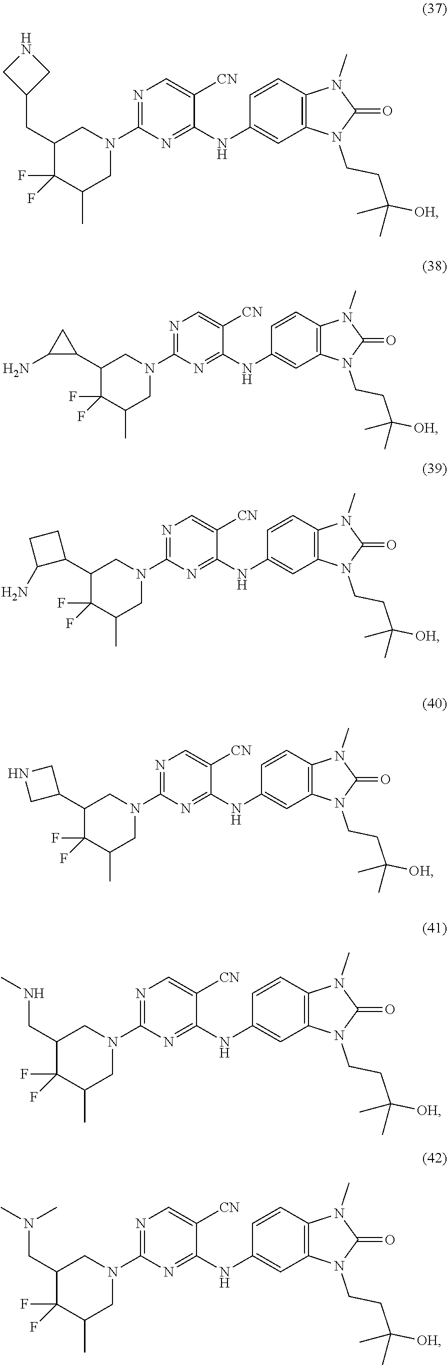 [SMILES: CC1CN(c2ncc(C#N)c(Nc3ccc4c(c3)n(CCC(C)(C)O)c(=O)n4C)n2)CC(CC2CNC2)C1(F)F ; CC1CN(c2ncc(C#N)c(Nc3ccc4c(c3)n(CCC(C)(C)O)c(=O)n4C)n2)CC(C2CC2N)C1(F)F ; CC1CN(c2ncc(C#N)c(Nc3ccc4c(c3)n(CCC(C)(C)O)c(=O)n4C)n2)CC(C2CCC2N)C1(F)F ; CC1CN(c2ncc(C#N)c(Nc3ccc4c(c3)n(CCC(C)(C)O)c(=O)n4C)n2)CC(C2CNC2)C1(F)F ; CNCC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CN(C)C)C1(F)F]

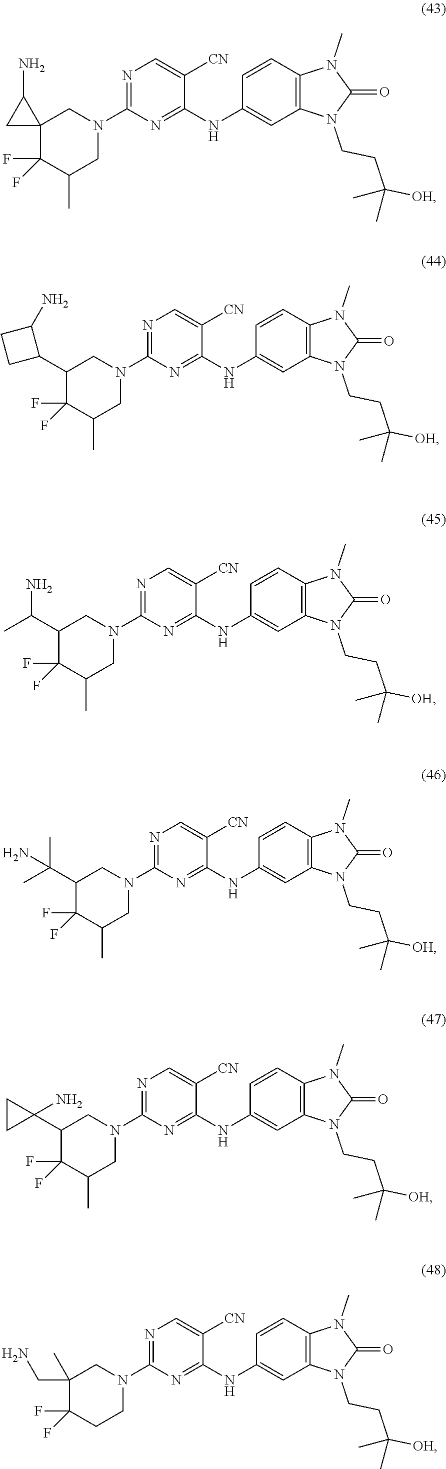 [SMILES: CC1CN(c2ncc(C#N)c(Nc3ccc4c(c3)n(CCC(C)(C)O)c(=O)n4C)n2)CC2(CC2N)C1(F)F ; CC1CN(c2ncc(C#N)c(Nc3ccc4c(c3)n(CCC(C)(C)O)c(=O)n4C)n2)CC(C2CCC2N)C1(F)F ; CC(N)C1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(C(C)(C)N)C1(F)F ; CC1CN(c2ncc(C#N)c(Nc3ccc4c(c3)n(CCC(C)(C)O)c(=O)n4C)n2)CC(C2(N)CC2)C1(F)F ; Cn1c(=O)n(CCC(C)(C)O)c2cc(Nc3nc(N4CCC(F)(F)C(C)(CN)C4)ncc3C#N)ccc21]

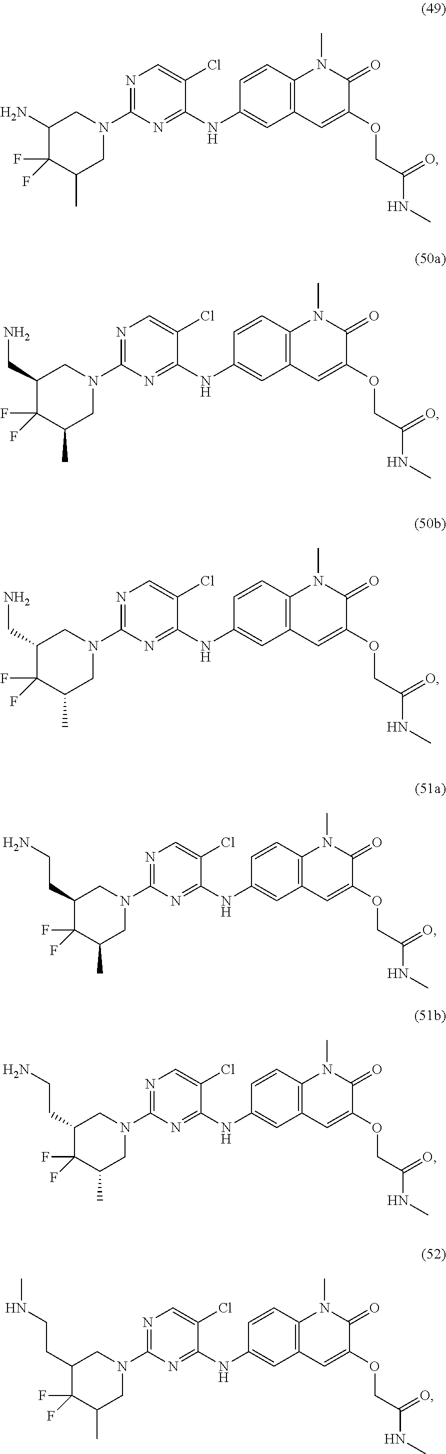 [SMILES: CNC(=O)COc1cc2cc(Nc3nc(N4CC(C)C(F)(F)C(N)C4)ncc3Cl)ccc2n(C)c1=O ; CNC(=O)COc1cc2cc(Nc3nc(N4C[C@H](CN)C(F)(F)[C@H](C)C4)ncc3Cl)ccc2n(C)c1=O ; CNC(=O)COc1cc2cc(Nc3nc(N4C[C@@H](CN)C(F)(F)[C@@H](C)C4)ncc3Cl)ccc2n(C)c1=O ; CNC(=O)COc1cc2cc(Nc3nc(N4C[C@H](CCN)C(F)(F)[C@H](C)C4)ncc3Cl)ccc2n(C)c1=O ; CNC(=O)COc1cc2cc(Nc3nc(N4C[C@@H](CCN)C(F)(F)[C@@H](C)C4)ncc3Cl)ccc2n(C)c1=O ; CNCCC1CN(c2ncc(Cl)c(Nc3ccc4c(c3)cc(OCC(=O)NC)c(=O)n4C)n2)CC(C)C1(F)F]

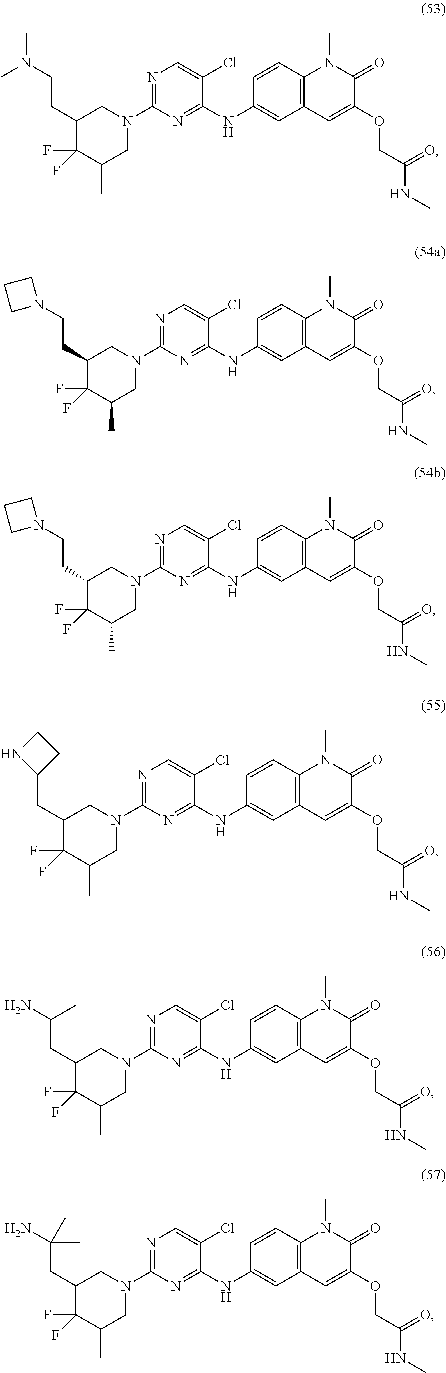

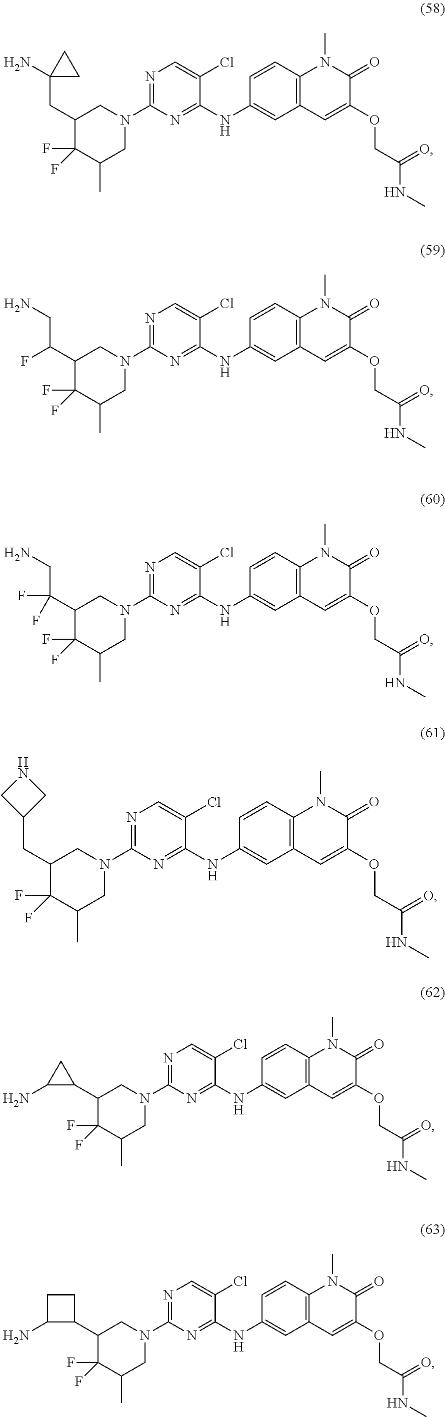 [SMILES: CNC(=O)COc1cc2cc(Nc3nc(N4CC(C)C(F)(F)C(CC5(N)CC5)C4)ncc3Cl)ccc2n(C)c1=O ; CNC(=O)COc1cc2cc(Nc3nc(N4CC(C)C(F)(F)C(C(F)CN)C4)ncc3Cl)ccc2n(C)c1=O ; CNC(=O)COc1cc2cc(Nc3nc(N4CC(C)C(F)(F)C(C(F)(F)CN)C4)ncc3Cl)ccc2n(C)c1=O ; CNC(=O)COc1cc2cc(Nc3nc(N4CC(C)C(F)(F)C(CC5CNC5)C4)ncc3Cl)ccc2n(C)c1=O ; CNC(=O)COc1cc2cc(Nc3nc(N4CC(C)C(F)(F)C(C5CC5N)C4)ncc3Cl)ccc2n(C)c1=O ; CNC(=O)COc1cc2cc(Nc3nc(N4CC(C)C(F)(F)C(C5CCC5N)C4)ncc3Cl)ccc2n(C)c1=O]

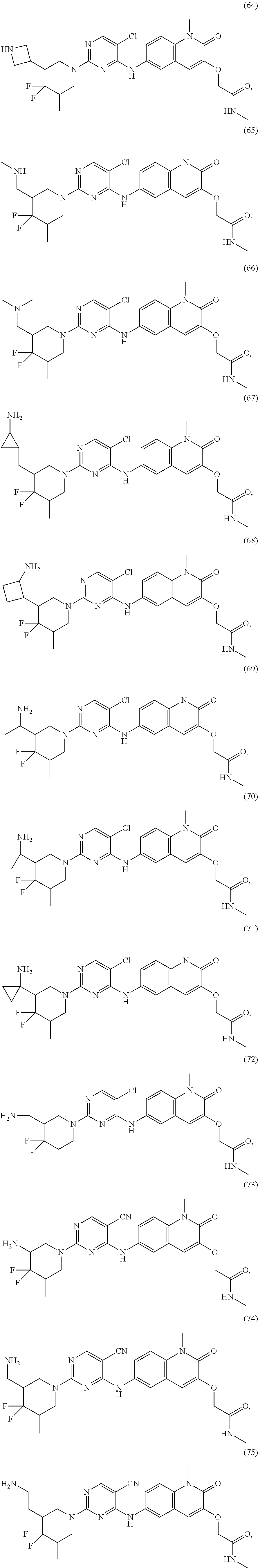

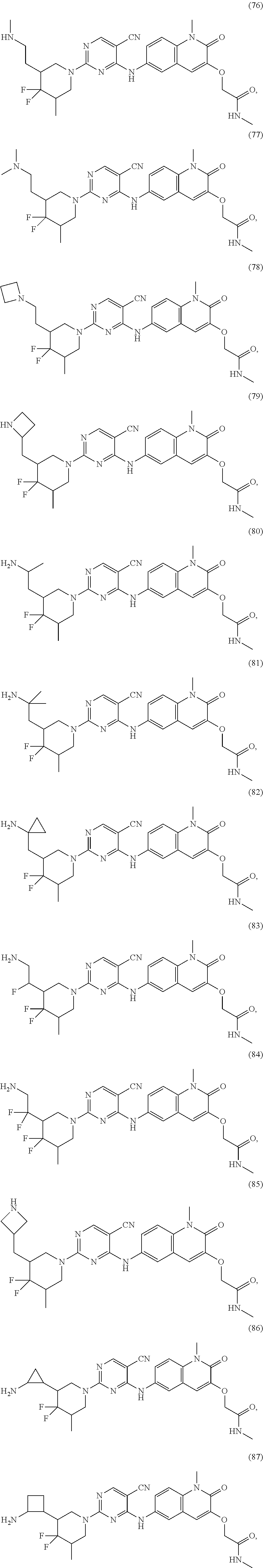

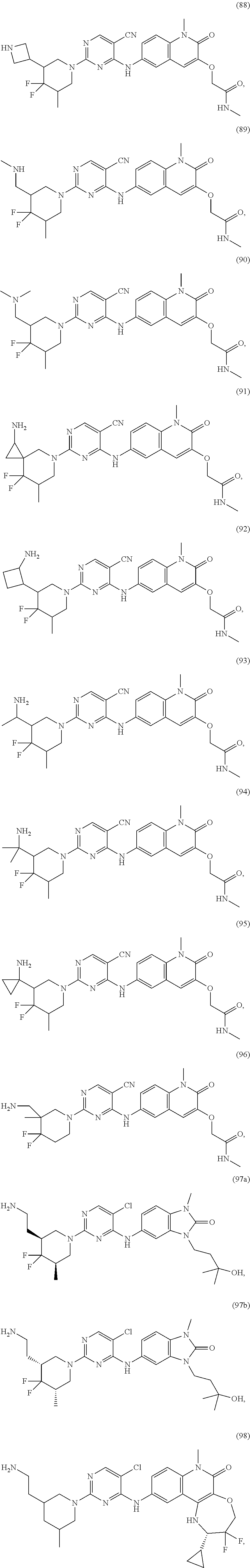

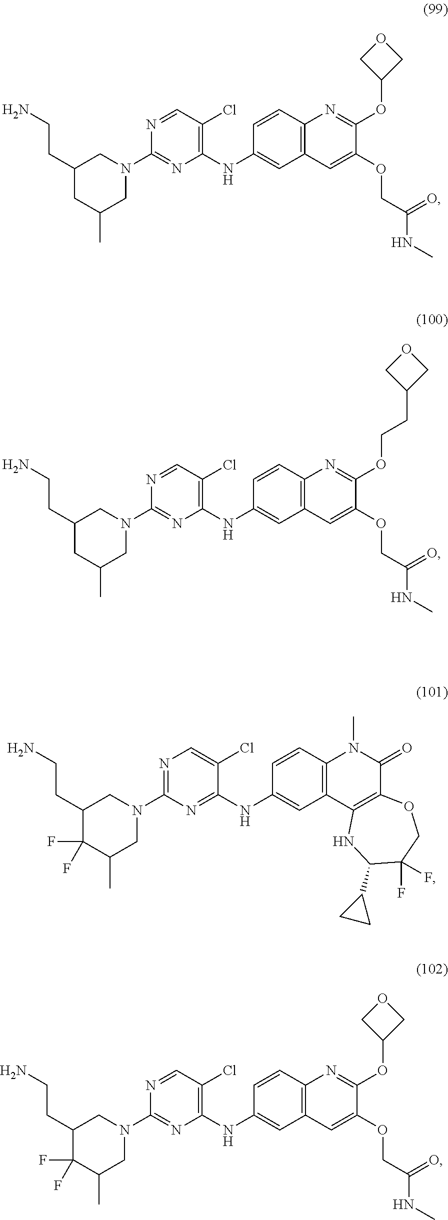 [SMILES: CNC(=O)COc1cc2cc(Nc3nc(N4CC(C)CC(CCN)C4)ncc3Cl)ccc2nc1OC1COC1 ; CNC(=O)COc1cc2cc(Nc3nc(N4CC(C)CC(CCN)C4)ncc3Cl)ccc2nc1OCCC1COC1 ; CC1CN(c2ncc(Cl)c(Nc3ccc4c(c3)c3c(c(=O)n4C)OCC(F)(F)[C@H](C4CC4)N3)n2)CC(CCN)C1(F)F ; CNC(=O)COc1cc2cc(Nc3nc(N4CC(C)C(F)(F)C(CCN)C4)ncc3Cl)ccc2nc1OC1COC1]

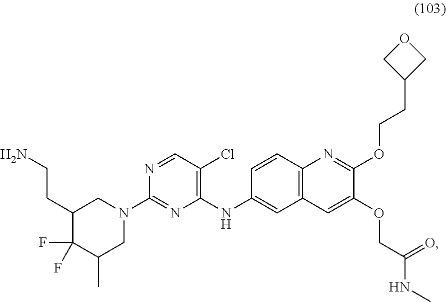 [SMILES: CNC(=O)COc1cc2cc(Nc3nc(N4CC(C)C(F)(F)C(CCN)C4)ncc3Cl)ccc2nc1OCCC1COC1]

or a pharmaceutically acceptable salt or stereoisomer thereof.

Compounds of the present disclosure may be in the form of a free acid or free base, or a pharmaceutically acceptable salt. A pharmaceutically acceptable salt of the compounds of this disclosure can be formed, for example, by reaction of an appropriate free base of a compound of the invention and an appropriate pharmaceutically acceptable acid in a suitable solvent under standard conditions well known in the art. See, for example, Gould, P. L., “Salt selection for basic drugs,” International Journal of Pharmaceutics, 33:201-217 (1986); Bastin, R. J., et al., “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19 (1977).

Compounds of the present disclosure may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. As used herein, the term “hydrogen”, i.e. H, refers to all isotopes of hydrogen, including protium (H) and deuterium (H). As used herein, the term “compound” embraces isotopic derivatives.

Compounds of formula (I) may also be in the form of N-oxides, crystalline forms (also known as polymorphs), co-crystals, active metabolites of the compounds having the same type of activity, prodrugs, tautomers, and unsolvated as well as solvated (e.g., hydrated) forms with pharmaceutically acceptable solvents such as water, ethanol, and the like, of the compounds. As used herein, the term “compound” embraces all these forms.

The compounds of formula (I) may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of a compound of formula (I). The term “co-crystal”, as used herein, refers to a stoichiometric multi-component system comprising a compound of formula (I) and a co-crystal former wherein the compound of formula (I) and the co-crystal former are connected by non-covalent interactions. The term “co-crystal former”, as used herein, refers to compounds which can form intermolecular interactions with a compound of formula (I) and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicylic acid, maleic acid, saccharin, 4,4′-bipyridine p-aminosalicylic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present disclosure is directed to a method for making a compound of formula (I), or a pharmaceutically acceptable salt or stereoisomer thereof. Broadly, the compounds of formula (I) or pharmaceutically-acceptable salts or stereoisomers thereof may be prepared by any process known to be applicable to the preparation of chemically related compounds. The compounds of formula (I) will be better understood in connection with the synthetic schemes that described in various working examples and which illustrate non-limiting methods by which the compounds of formula (I) may be prepared.

### Pharmaceutical Compositions

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds of formula (I) are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin.

See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., ChemicalMeans of Transdermal Drug Permeation Enhancement in Transdermal and TopicalDrug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof that is effective in producing the desired therapeutic response in a particular patient suffering from a disease or disorder mediated by aberrant BCL6 activity. The term “therapeutically effective amount” thus includes the amount of the compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of BCL6 in diseased cells.

The total daily dosage of the compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001.

### Methods of Use

In some aspects, the present disclosure is directed to treating diseases or disorders that involves (e.g., characterized or mediated by) aberrant (e.g., elevated levels of BCL6 or otherwise functionally abnormal e.g., deregulated BCL6 levels) BCL6 activity relative to a non-pathological state. The methods entail administering a therapeutically effective amount of a compound of formula (I) or a pharmaceutically acceptable salt or stereoisomer thereof, to a subject in need thereof.

The term “subject” (or “patient”) as used herein includes all members of the animal kingdom prone to or suffering from the cancer. In some embodiments, the subject is a mammal, e.g., a human or a non-human mammal. The methods are also applicable to companion animals such as dogs and cats. A subject “in need of” treatment according to the present disclosure may be “suffering from or suspected of suffering from” a specific cancer may have been positively diagnosed or otherwise presents with a sufficient number of risk factors or a sufficient number or combination of signs or symptoms such that a medical professional could diagnose or suspect that the subject is suffering from the cancer. Thus, subjects suffering from a specific disease or disorder, and subjects suspected of suffering from a specific disease or disorder are not necessarily two distinct groups.

In some embodiments, the methods are directed to treating subjects having cancer. The methods embrace treatment of adult tumors/cancers and pediatric tumors/cancers. The cancers may be vascularized, or not yet substantially vascularized, or non-vascularized tumors.

In some embodiments, the cancer is a lymphoid malignancy.

In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

In some embodiments, methods of the present disclosure entail treatment of subjects having cell proliferative diseases or disorders of the hematological system.

In some embodiments, the methods are directed to treating subjects having an inflammatory disease or disorder.

In some embodiments, the inflammatory disease or disorder is inflammatory bowel disease, myocarditis, endometriosis, atherosclerosis, an allergic disease or disorder, or an autoimmune disease or disorder. In some embodiments, the allergic disease or disorder is asthma or pollinosis. In some embodiments, the autoimmune disease is noninfectious meningitis, autoimmune encephalitis, transverse myelitis, or acute disseminated encephalomyelitis.

Compounds of formula (I) may be administered to a cancer patient as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments.

Therapy may also be given to patients who have had previous treatments which were unsuccessful or partially successful but who became unresponsive or intolerant to the particular treatment.

Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present disclosure may entail administration of a compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of 5 days.

### Combination Therapy

The compounds of formula (I) and their pharmaceutically acceptable salts and stereoisomers may be used in combination or concurrently with at least one other active agent, e.g., anti-cancer agent or regimen, in treating cancer. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) in combination with one or more additional therapeutics known for use in treating cancer. The dosage of the additional therapeutic may be the same or even lower than known or recommended doses. See, Hardman et a/., eds., Goodman & Gilman's the Pharmacological Basis of Basis of Therapeutics, 10th ed., McGraw-Hill, New York, 2001; Physician's Desk Reference 60th ed., 2006. Anti-cancer agents that may be suitable for use in combination with the compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, a compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a compound of the present disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments, a compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the compounds of the present disclosure may be used in combination with other anti-cancer agents, examples of which include Etoposide (e.g., lymphomas, and non-lymphocytic leukemia), Vincristine (e.g., leukemia), Daunorubicin (e.g., acute myeloid leukemia (AML), acute lymphocytic leukemia (ALL), chronic myelogenous leukemia (CML), and Kaposi's sarcoma), Rituximab (e.g., non-Hodgkin's lymphoma), Alemtuzumab (e.g., chronic lymphocytic leukemia (CLL), cutaneous T-cell lymphoma (CTCL) and T-cell lymphoma), Bortezomib (e.g., multiple myeloma and mantle cell lymphoma), Pegaspargase (e.g., acute lymphoblastic leukemia), Keytruda© (e.g., Hodgkin lymphoma), and dexamethasone (e.g., acute multiple myeloma).

In some embodiments, the additional anti-cancer agent is an enhancer of zeste homolog 2 (EZH2) inhibitor, examples of which include tazemetostat, GSK126, lirametostat (CPI-1205), CPI-0209, PF-06821497, SHR2554, HH2853, valemetostat (DS3201), MAK-683, and FTX-6058.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1: Synthesis of 5-((2-((3S,5R)-3-(2-aminoethyl)-4,4-difluoro-5-methylpiperidin-1-yl)-5-chloropyrimidin-4-yl)amino)-3-(3-hydroxy-3-methylbutyl)-1-methyl-1H-benzo[d]imidazol-2(3H)-one (97a) and 5-((2-((3R,5S)-3-(2-aminoethyl)-4,4-difluoro-5-methylpiperidin-1-yl)-5-chloropyrimidin-4-yl)amino)-3-(3-hydroxy-3-methylbutyl)-1-methyl-1H-benzo[d]imidazol-2(3H)-one (97b)

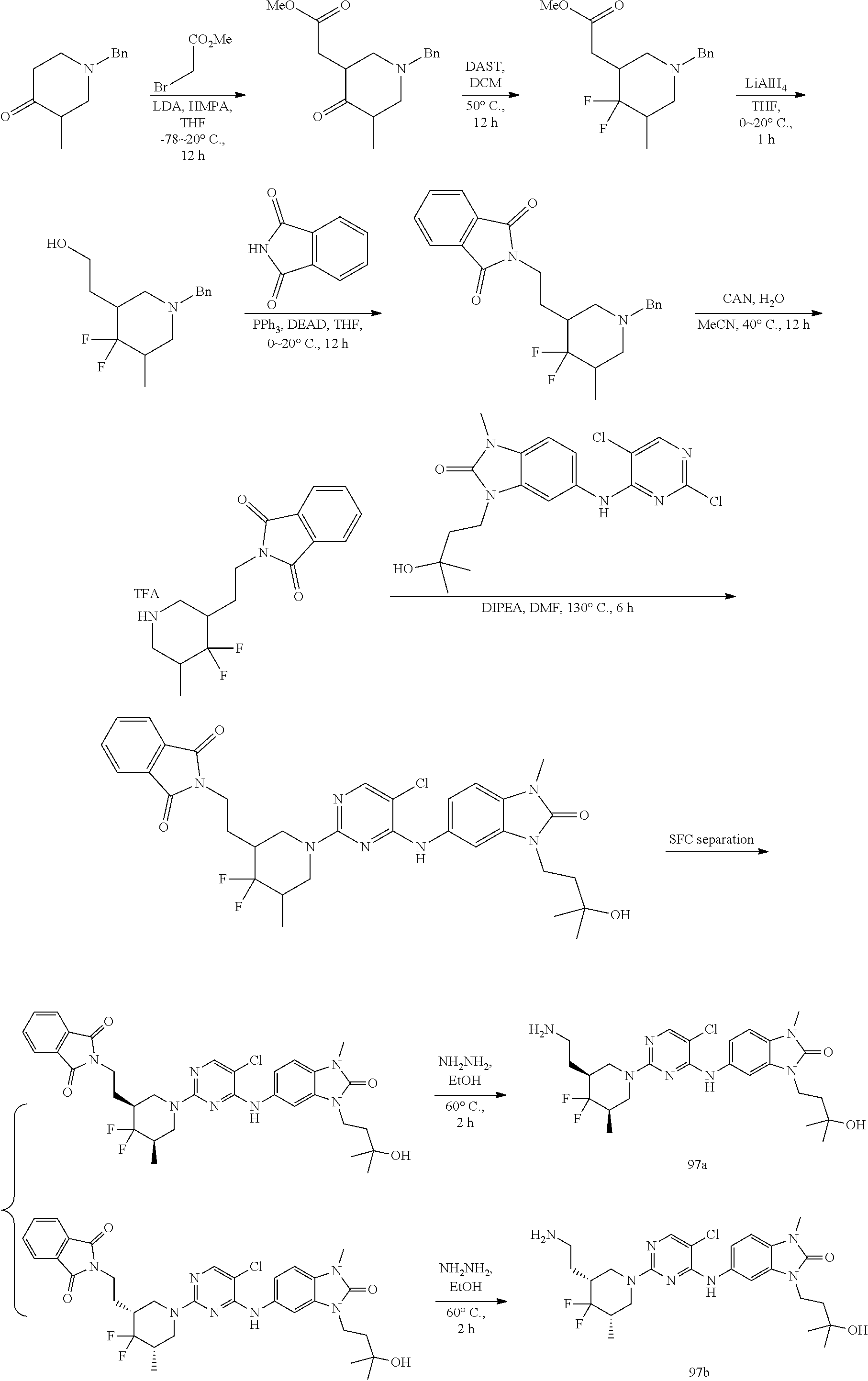

### Methyl 2-(1-benzyl-5-methyl-4-oxo-3-piperidyl)acetate

To a solution of 1-benzyl-3-methyl-piperidin-4-one (30 g, 147.58 mmol, 1 eq) in THF (300 mL) was added dropwise LDA (2 M, 73.80 mL, 1 eq) at −78° C. After addition, the mixture was stirred at −78° C. for 30 min, and then methyl 2-bromoacetate (33.86 g, 221.38 mmol, 20.90 mL, 1.5 eq) and HMPA (31.74 g, 177.10 mmol, 31.12 mL, 1.2 eq) was added dropwise at −78° C., and the mixture was stirred at −78° C. for 2.5 hr. Then the resulting mixture was stirred at 20° C. for 9 hr. The reaction mixture was quenched by adding NH₄Cl (100 mL, aq.), and then diluted with H₂O (500 mL) and extracted with EtOAc (300 mL×3). The combined organic layers were washed with brine (200 mL), dried over Na₂SO₄, filtered and concentrated under reduced pressure to give a residue which was purified by column chromatography (SiO₂, Petroleum ether/Ethyl acetate=I/O to 0/1) to give the title compound as a yellow oil (8.3 g, 17%). LCMS: [M+1]⁺=276.2.

### Methyl 2-(1-benzyl-4,4-difluoro-5-methyl-3-piperidyl)acetate

To a solution of methyl 2-(1-benzyl-5-methyl-4-oxo-3-piperidyl)acetate (8.3 g, 30.14 mmol, 1 eq) in DCM (85 mL) was added DAST (77.74 g, 482.31 mmol, 63.72 mL, 16 eq). The mixture was stirred at 50° C. for 12 hr. The residue was diluted with DCM (60 mL) and then the mixture was added dropwise to sat. NaHCO₃ aq. (200 mL). The resulting mixture was stirred at 20° C. for 20 min, then extracted with EtOAc (100 mL×3). The combined organic layers were washed with brine (50 mL), dried over Na₂SO₄, filtered and concentrated under reduced pressure to give a residue which was purified by column chromatography (SiO₂, Petroleum ether/Ethyl acetate=1/0 to 0/1) to give the title compound as a yellow oil (3.7 g, 41%). ¹H NMR (400 MHz, CDCl₃) δ=7.31-7.21 (m, 5H), 3.64-3.60 (m, 3H), 3.57-3.54 (m, 1H), 3.42 (d, J=13.2 Hz, 1H), 2.97-2.87 (m, 1H), 2.78-2.66 (m, 2H), 2.65-2.48 (m, 1H), 2.25-2.05 (m, 2H), 2.04-1.87 (m, 2H), 0.92 (d, J=6.8 Hz, 3H).

### 2-(1-Benzyl-4,4-difluoro-5-methyl-3-piperidyl)ethanol

To a solution of methyl 2-(1-benzyl-4,4-difluoro-5-methyl-3-piperidyl)acetate (1 g, 3.36 mmol, 1 eq) in THE (10 mL) was added LiAlH₄ (255.29 mg, 6.73 mmol, 2 eq) dropwise at 0° C. slowly, then the mixture was stirred at 20° C. for 1 hr. The reaction mixture was quenched by addition NH₄Cl (10 mL, aq.), and then diluted with H₂O (10 mL) and extracted with EtOAc (10 mL×3). The combined organic layers were washed with brine (10 mL), dried over Na₂SO₄, filtered and concentrated under reduced pressure to give the title compound as a yellow oil (700 mg, 77%). LCMS: [M+1]⁺=270.1.

### 2-[2-(1-Benzyl-4,4-difluoro-5-methyl-3-piperidyl)ethyl]isoindoline-1,3-dione

To a solution of 2-(1-benzyl-4,4-difluoro-5-methyl-3-piperidyl)ethanol (700 mg, 2.60 mmol, 1 eq) and isoindoline-1,3-dione (458.88 mg, 3.12 mmol, 1.2 eq) in DCM (7 mL) was added PPh₃ (886.20 mg, 3.38 mmol, 1.3 eq) at 0° C. A solution of DEAD (588.43 mg, 3.38 mmol, 614.23 μL, 1.3 eq) in DCM (1 mL) was then added dropwise at 0° C. The resulting reaction mixture was stirred at 20° C. for 12 hr. The reaction mixture was concentrated under reduced pressure and the residue was purified by column chromatography (SiO₂, Petroleum ether/Ethyl acetate=1/0 to 0/1) to give the title compound as a colorless oil (600 mg, 58%). LCMS: [M+1]⁺=399.1.

### 2-[2-(4,4-Difluoro-5-methyl-3-piperidyl)ethyl]isoindoline-1,3-dione

To a solution of 2-[2-(1-benzyl-4,4-difluoro-5-methyl-3-piperidyl)ethyl]isoindoline-1,3-dione (200 mg, 501.95 μmol, 1 eq) in MeCN (8 mL) and H₂O (2 mL) was added ceric ammonium nitrate (2.75 g, 5.02 mmol, 2.50 mL, 10 eq). The mixture was stirred at 40° C. for 12 hr. The reaction mixture was concentrated under reduced pressure and the residue was purified by reversed-phase HPLC (Column: 80g Agela C18, Solvent for sample dissolution: about 0.20 grams of sample dissolved in 2 ml of MeOH; Flow rate: 40 ml/min; Mobile phase: TFA; Gradient B %:30-60% 10 min; 60% 5 min; Instrument: Biotage) to give the title compound as a yellow solid (140 mg, 66%, TFA salt). LCMS: [M+1]⁺=309.2.

### 2-[2-[1-[5-Chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione

To a solution of 5-[(2,5-dichloropyrimidin-4-yl)amino]-3-(3-hydroxy-3-methyl-butyl)-1-methyl-benzimidazol-2-one (160 mg, 403.76 μmol, 1 eq) and 2-[2-(4,4-difluoro-5-methyl-3-piperidyl)ethyl]isoindoline-1,3-dione (139.21 mg, 403.76 μmol, 1 eq, TFA) in DMF (3 mL) was added DIPEA (156.55 mg, 1.21 mmol, 210.99 μL, 3 eq). The mixture was stirred at 130° C. for 6 hr. The reaction mixture was concentrated under reduced pressure and the residue was purified by prep-HPLC (column: Phenomenex Gemini-NX 80*40 mm*3 μm; mobile phase: [water(10 mM NH₄HCO₃)—MeCN]; B %: 30%-60%, 8 min) to give the title compound as a yellow solid (200 mg, 74.%). LCMS: [M+1]⁺=668.1.

### 2-[2-[(3S,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione and 2-[2-[(3R,5S)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione

An amount of 200 mg of the mixture of the enantiomers was separated by SFC (supercritical fluid chromatography) on a column: DAICEL CHIRALPAK AD (250 mm*30 mm, 10 μm); mobile phase: [0.1% NH₃H₂O ETOH]; B %: 46%-46%, 7 min. 2-[2-[(3S,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione (50 mg, 44% yield, 96% purity) was obtained as a white solid and 2-[2-[(3R,5S)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione (60 mg, 54% yield, 99% purity) was obtained as a white solid.

### 5-[[2-[(3S,5R)-3-(2-aminoethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (97a)

To a solution of 2-[2-[(3S,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione (50.00 mg, 74.84 μmol, 1 eq) in EtOH (0.5 mL) was added NH₂NH₂·H₂O (56.19 mg, 1.12 mmol, 54.56 μL, 15 eq). The mixture was stirred at 60° C. for 1 hr.

The reaction mixture was concentrated under reduced pressure and the residue was purified by prep-HPLC (column: Waters Xbridge BEH C18 100*30 mm*10 μm; mobile phase: [water(10 mM NH₄HCO₃)—MeCN]; B %: 30%-55%, 8 min) to give the title compound as a white solid (15.2 mg, 37%). ¹H NMR (400 MHz, MeOD) δ=7.94 (s, 1H), 7.39 (dd, J=1.9, 8.4 Hz, 1H), 7.34 (d, J=1.6 Hz, 1H), 7.15 (d, J=8.6 Hz, 1H), 4.66 (br d, J=13.4 Hz, 1H), 4.53 (br d, J=13.4 Hz, 1H), 4.07-3.98 (m, 2H), 3.44 (s, 3H), 2.82-2.55 (m, 4H), 2.05-1.82 (m, 5H), 1.45-1.34 (m, 1H), 1.28 (s, 6H), 0.99 (d, J=6.8 Hz, 3H). LCMS: [M+1]⁺=538.2.

### 5-[[2-[(3R,5S)-3-(2-aminoethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (97b)

To a solution of 2-[2-[(3R,5S)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]ethyl]isoindoline-1,3-dione (50 mg, 74.84 μmol, 1 eq) in EtOH (0.5 mL) was added NH₂NH₂·H₂O (56.19 mg, 1.12 mmol, 54.56 μL, 15 eq). The mixture was stirred at 60° C. for 2 hr. The reaction mixture was concentrated under reduced pressure and the residue was purified by prep-HPLC (column: Waters Xbridge BEH C18 100*30 mm*10 μm; mobile phase: [water (10 mM NH₄HCO₃)—MeCN]; B %: 30%-55%, 8 min) to give the title compound as a white solid (20.1 mg, 49%). ¹H NMR (400 MHz, MeOD) δ=7.94 (s, 1H), 7.39 (dd, J=1.6, 8.4 Hz, 1H), 7.34 (s, 1H), 7.15 (d, J=8.0 Hz, 1H), 4.66 (br d, J=14.0 Hz, 1H), 4.53 (br d, J=13.2 Hz, 1H), 4.09-3.98 (m, 2H), 3.44 (s, 3H), 2.75-2.58 (m, 4H), 1.99-1.79 (m, 5H), 1.44-1.34 (m, 1H), 1.28 (s, 6H), 0.99 (d, J=6.8 Hz, 3H). LCMS: [M+1]⁺=538.2.

The absolute configurations of compounds 97a & 97b were randomly assigned based on the alkylamino group and methyl group being in cis-conformation.

### Example 2: Synthesis of 5-[[2-[(3S,5R)-3-(aminomethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (2a) 5-[[2-[(3R,5S)-3-(aminomethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (2b)

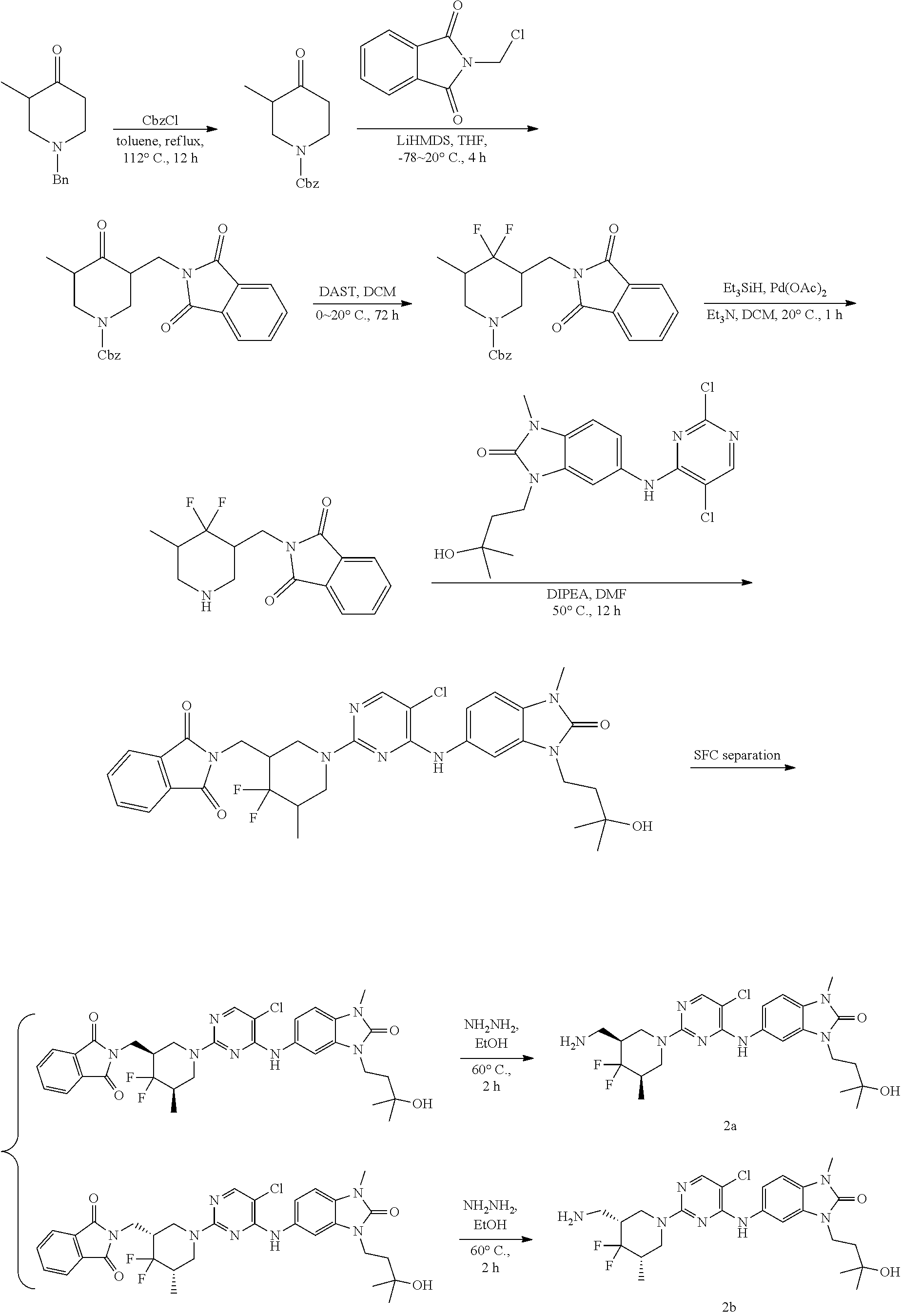

### Benzyl 3-methyl-4-oxo-piperidine-1-carboxylate

To a solution of 1-benzyl-3-methyl-piperidin-4-one (26 g, 127.90 mmol, 1 eq) in toluene (300 mL) was added CbzCl (33.38 g, 195.69 mmol, 27.82 mL, 1.53 eq). The mixture was stirred at 112° C. for 12 hr. The residue was diluted with H₂O (200 mL) and extracted with EtOAc (100 mL×3). The combined organic layers were washed with brine (100 mL), dried over Na₂SO₄, filtered and concentrated under reduced pressure to give a residue which was purified by column chromatography (SiO₂, Petroleum ether/Ethyl acetate=1/0 to 0/1) to give the title compound as a yellow oil (25 g, 79%). LCMS: [M+1]⁺=248.2.

### Benzyl 3-[(1,3-dioxoisoindolin-2-yl)methyl]-5-methyl-4-oxo-piperidine-1-carboxylate

To a solution of benzyl 3-methyl-4-oxo-piperidine-1-carboxylate (10 g, 40.44 mmol, 1 eq) in THF (200 mL) was added LiHMDS (1 M, 50.55 mL, 1.25 eq) dropwise over 30 min at −78° C. After addition, the mixture was stirred at −78° C. for 90 min, then 2-(chloromethyl)isoindoline-1,3-dione (11.86 g, 60.66 mmol, 1.5 eq) in THF (200 mL) was added dropwise at −78° C. The resulting mixture was stirred at −78° C. for 2 hr. The reaction mixture was quenched by addition NH₄Cl (200 mL, aq.) at 0° C., and then diluted with H₂O (200 mL) and extracted with EtOAc (100 mL×3). The combined organic layers were washed with brine (100 mL), dried over Na₂SO₄, filtered and concentrated under reduced pressure to give a residue which was purified by reversed-phase HPLC (Column: 800g Agela C18; Solvent for sample dissolution: about 8.50 grams of sample dissolved in 40 ml of MeOH; Flow rate: 120 ml/min; Mobile phase: water-MeCN; Gradient B %:45-75% 30 min; 75% 30 min; Instrument: Biotage) to give the title compound as a white solid (5 g, 30%). LCMS: [M+1]⁺=407.1.

### Benzyl 3-[(1,3-dioxoisoindolin-2-yl)methyl]-4,4-difluoro-5-methyl-piperidine-1-carboxylate

To a solution of benzyl 3-[(1,3-dioxoisoindolin-2-yl)methyl]-5-methyl-4-oxo-piperidine-1-carboxylate (4 g, 9.84 mmol, 1 eq) in DCM (50 mL) was added DAST (25.38 g, 157.47 mmol, 20.81 mL, 16 eq) at 0° C. Then the resulting mixture was stirred at 20° C. for 72 hr. The residue was diluted with DCM (40 mL) and then the mixture was added dropwise to sat. NaHCO₃ aq. (100 mL). The resulting mixture was stirred at 20° C. for 20 min, and then extracted with EtOAc (50 mL×3). The combined organic layers were washed with brine (50 mL), dried over Na₂SO₄, filtered and concentrated under reduced pressure to give a residue which was purified by reversed-phase HPLC (Column: 330g Agela C18; Solvent for sample dissolution: about 5.00 grams of sample dissolved in 20 ml of MeOH; Flow rate: 70 ml/min; Mobile phase: water MeCN; Gradient B %:45-75% 25 min; 75% 40 min; Instrument: Biotage) to give the title compound as a yellow oil (1.7 g, 40%). ¹H NMR (400 MHz, CDCl₃) δ=7.93-7.69 (m, 4H), 7.27 (s, 5H), 5.19-5.00 (m, 2H), 4.27-4.16 (m, 1H), 4.12-4.05 (m, 1H), 3.83-3.70 (m, 1H), 2.98-2.61 (m, 2H), 2.56-2.30 (m, 1H), 2.05-1.84 (m, 1H), 1.59 (br s, 1H), 1.05 (d, J=6.6 Hz, 3H).

### 2-[(4,4-Difluoro-5-methyl-3-piperidyl)methyl]isoindoline-1,3-dione

To a solution of benzyl 3-[(1,3-dioxoisoindolin-2-yl)methyl]-4,4-difluoro-5-methyl-piperidine-1-carboxylate (1.5 g, 3.50 mmol, 1 eq) in DCM (5 mL) was added TEA (1.06 g, 10.50 mmol, 1.46 mL, 3 eq), Pd(OAc)₂ (314.42 mg, 1.40 mmol, 0.4 eq) and Et₃SiH (2.44 g, 21.01 mmol, 3.36 mL, 6 eq). The mixture was stirred at 20° C. for 1 hr. The reaction mixture was concentrated under reduced pressure and the residue was purified by reversed-phase HPLC (Column: 330g Agela C18; Solvent for sample dissolution: about 5.00 grams of sample dissolved in 20 ml of MeOH; Flow rate: 70 ml/min; Mobile phase: water-MeCN; Gradient B %:30-60% 25 min; 60% 10 min; Instrument: Biotage) to give the title compound as a yellow solid (800 mg, 78%). LCMS: [M+1]⁺=295.0.

### 2-[[1-[5-Chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione

To a solution of 5-[(2,5-dichloropyrimidin-4-yl)amino]-3-(3-hydroxy-3-methyl-butyl)-1-methyl-benzimidazol-2-one (350 mg, 883.24 μmol, 1 eq) and 2-[(4,4-difluoro-5-methyl-3-piperidyl)methyl]isoindoline-1,3-dione (259.93 mg, 883.24 μmol, 1 eq) in DMF (2 mL) was added DIPEA (342.46 mg, 2.65 mmol, 461.53 μL, 3 eq). The reaction mixture was stirred at 50° C. for 12 hr. The reaction mixture was concentrated under reduced pressure and the residue was purified by prep-HPLC (column: Phenomenex Gemini-NX 80*40 mm*3 μm; mobile phase: [water (10 mM NH₄HCO₃)—MeCN]; B %: 40%-60%, 8 min) to give the title compound as a yellow solid (170 mg, 28% yield, 95% purity). ¹H NMR (400 MHz, MeOD) δ=7.86 (s, 1H), 7.71 (br s, 3H), 7.34 (d, J=1.6 Hz, 1H), 7.25 (br d, J=8.4 Hz, 1H), 6.95 (br d, J=8.4 Hz, 1H), 4.66-4.50 (m, 2H), 4.09-3.86 (m, 3H), 3.73-3.67 (m, 1H), 3.45-3.36 (m, 3H), 2.89 (br t, J=12.8 Hz, 1H), 2.74 (br t, J=12.8 Hz, 1H), 2.47-2.28 (m, 1H), 2.11-1.90 (m, 1H), 1.86-1.75 (m, 1H), 1.89-1.74 (m, 2H), 1.28 (d, J=1.2 Hz, 6H), 1.02 (d, J=6.8 Hz, 3H).

### 2-[[(3S,5S)-1-[5-Chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione and 2-[[(3R,5R))-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione

170 mg of the mixture of enantiomers was separated by SFC (supercritical fluid chromatography) on a column: REGIS(S,S)WHELK-01(250 mm*25 mm, 10 μm); mobile phase: [Neu-EtOH]; B %: 50%-50%, 8 min to give 2-[[(3S,5S)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione as a white solid (70 mg, 40% yield, 96% purity) and 2-[[(3R,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione as a white solid (70 mg, 40% yield, 96% purity).

### 5-[[2-[(3S,5R)-3-(aminomethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (2a)

To a solution of 2-[[(3R,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione (80 mg, 122.30 μmol, 1 eq) in EtOH (2 mL) was added NH₂NH₂·H₂O (91.84 mg, 1.83 mmol, 89.16 μL, 15 eq). The mixture was stirred at 60° C. for 2 hr.

The reaction mixture was concentrated under reduced pressure and the residue was purified by prep-HPLC (column: Waters Xbridge BEH C18 100*30 mm*10 μm; mobile phase: [water(10 mM NH₄HCO₃)—MeCN]; B %: 25%-55%, 8 min) to give the title compound as a white solid (16.7 mg, 26.%). ¹H NMR (400 MHz, MeOD) δ=7.95 (s, 1H), 7.44-7.29 (m, 2H), 7.12 (br d, J=8.4 Hz, 1H), 4.75 (br d, 0.1=13.2 Hz, 1H), 4.52 (br d, 0.1=13.4 Hz, 1H), 4.07-3.97 (m, 2H), 3.43 (s, 3H), 3.03 (br dd, J=4.4, 13.4 Hz, 1H), 2.73 (q, J=13.2 Hz, 2H), 2.59-2.54 (m, 1H), 2.03-1.83 (m, 4H), 1.28 (s, 6H), 0.99 (br d, J=6.4 Hz, 3H). LCMS: [M+1]⁺=524.2.

### 5-[[2-[(3R,5S)-3-(Aminomethyl)-4,4-difluoro-5-methyl-1-piperidyl]-5-chloro-pyrimidin-4-yl]amino]-3-(3-hydroxy-3-methyl-butyl)-1-methyl-benzimidazol-2-one (2b)

To a solution of 2-[[(3S,5S)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]methyl]isoindoline-1,3-dione (80 mg, 122.30 μmol, 1 eq) in EtOH (2 mL) was added NH₂NH₂. H₂O (91.84 mg, 1.83 mmol, 89.16 μL, 15 eq). The reaction mixture was stirred at 60° C. for 2 hr. The reaction mixture was concentrated under reduced pressure and the residue was purified by prep-HPLC (column: Waters Xbridge BEH C18 100*30 mm*10 μm; mobile phase: [water (10 mM NH₄HCO₃)—MeCN]; B %: 25%-55%, 8 min) to give the title compound as a white solid (16.6 mg, 256%). ¹H NMR (400 MHz, MeOD) δ=7.95 (s, 1H), 7.40 (br d, J=8.4 Hz, 1H), 7.33 (s, 1H), 7.12 (br d, J=8.4 Hz, 1H), 4.76 (br d, J=13.2 Hz, 1H), 4.52 (br d, J=13.4 Hz, 1H), 4.08-3.98 (m, 2H), 3.43 (s, 3H), 3.03 (br dd, J=4.4, 13.4 Hz, 1H), 2.73 (q, J=13.2 Hz, 2H), 2.59-2.54 (m, 1H), 2.03-1.83 (m, 4H), 1.28 (s, 6H), 0.99 (br d, =6.4 Hz, 3H). LCMS: [M+1]⁺=524.2.

The absolute configurations of compounds 2a & 2b were randomly assigned based on the alkylamino group and methyl group being in cis-conformation.

### Example 3: Synthesis of 2-((6-((2-((3S,5R)-3-(aminomethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (50a) 2-((6-((2-((3R,5S)-3-(aminomethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (50b)

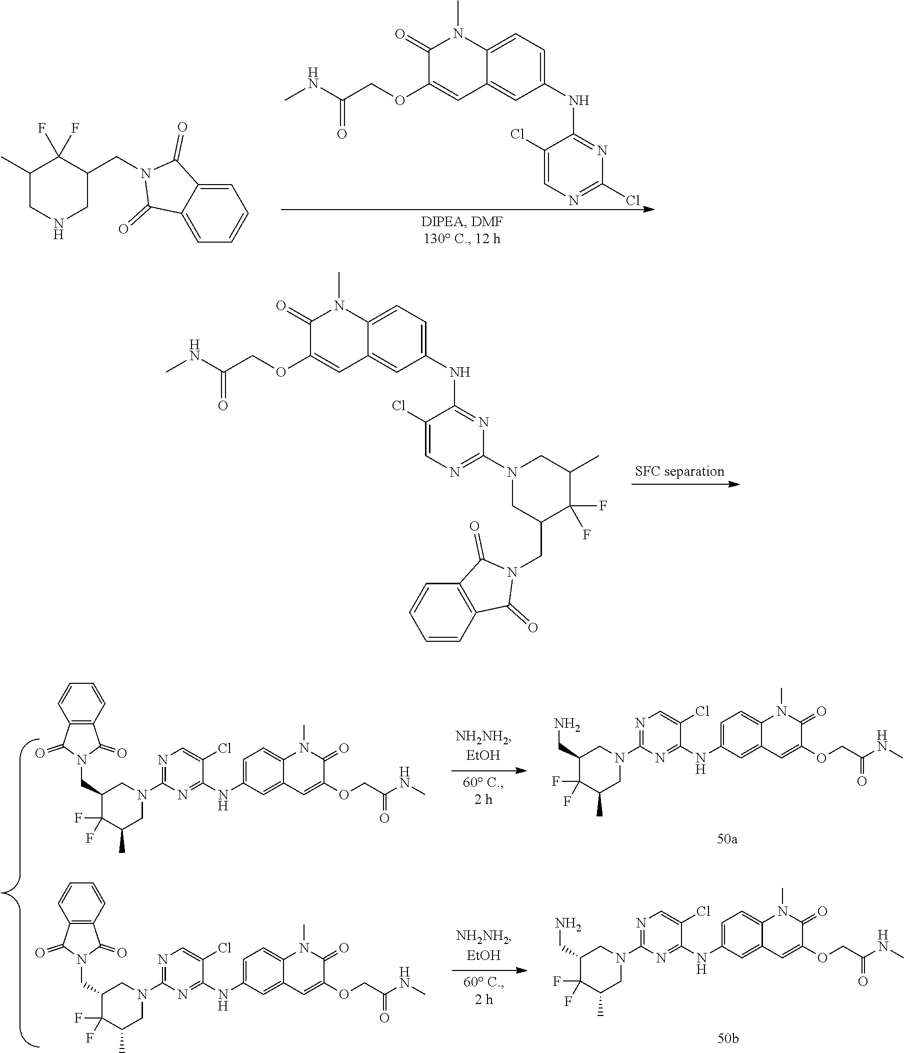

### 2-((6-((S-Chloro-2-(3-((1,3-dioxoisoindolin-2-yl)methyl)-4,4-difluoro-5-methylpiperidin-1-yl)pyrimidin-4-yl)amino)-1-methyl-2-oxo-1,2-dihydroquinolin-3-yl)oxy)-N-methylacetamide

To a solution of 2-[[6-[(2,5-dichloropyrimidin-4-yl)amino]-1-methyl-2-oxo-3-quinolyl]oxy]-N-methyl-acetamide (350 mg, 857.34 μmol, 1 eq) and 2-[(4,4-difluoro-5-methyl-3-piperidyl)methyl]isoindoline-1,3-dione (302.77 mg, 1.03 mmol, 1.2 eq) in DMF (3 mL) was added DIPEA (332.42 mg, 2.57 mmol, 448.00 μL, 3 eq). The reaction mixture was stirred at 130° C. for 12 hr. The reaction mixture was concentrated under reduced pressure and the residue was purified by prep-HPLC (column: Phenomenex Gemini-NX 80*40 mm*3 μm; mobile phase: [water(10 mM NH₄HCO₃)—MeCN]; B %: 25%-55%, 8 min) to give the title compound as a yellow solid (260 mg, 46%). ¹H NMR (400 MHz, DMSO-d₆) δ=8.91 (s, 1H), 8.05 (s, 1H), 7.91 (br d, J=4.0 Hz, 1H), 7.86 (br d, J=2.0 Hz, 1H), 7.78 (br s, 3H), 7.71-7.63 (m, 1H), 7.38 (br d, J=7.6 Hz, 1H), 7.15 (s, 1H), 4.66-4.44 (m, 4H), 3.94-3.89 (m, 1H), 3.64 (s, 3H), 3.60-3.50 (m, 1H), 2.84 (br t, J=12.8 Hz, 1H), 2.72-2.66 (m, 2H), 2.64 (d, J=4.8 Hz, 3H), 2.13 (br s, 1H), 0.94 (br d, J=6.8 Hz, 3H).

### 2-((6-((5-Chloro-2-((3R,5R)-3-((1, 3-dioxoisoindolin-2-yl)methyl)-4, 4-difluoro-5-methylpiperidin-1-yl)pyrimidin-4-yl)amino)-1-methyl-2-oxo-1,2-dihydroquinolin-3-yl)oxy)-N-methylacetamide and 2-((6-((5-chloro-2-((3S,5S)-3-((1,3-dioxoisoindolin-2-y)methyl)-4,4-difluoro-5-methylpiperidin-1-yl)pyrimidin-4-yl)amino)-1-methyl-2-oxo-1,2-dihydroquinolin-3-yl)oxy)-N-methylacetamide

260 mg of the mixture of enantiomers was separated by SFC (supercritical fluid chromatography) on a column: DAICEL CHIRALPAK AD (250 mm*30 mm, 10 μm); mobile phase: [Neu-IPA]; B %: 42%-42%, 12 min to give 2-[[6-[[5-chloro-2-[(3R,5R)-3-[(1,3-dioxoisoindolin-2-yl)methyl]-4,4-difluoro-5-methyl-1-piperidyl]pyrimidin-4-yl]amino]-1-methyl-2-oxo-3-quinolyl]oxy]-N-methyl-acetamide as a yellow solid (75 mg, 32%) and 2-[[6-[[5-chloro-2-[(3S,5S)-3-[(1,3-dioxoisoindolin-2-yl)methyl]-4,4-difluoro-5-methyl-1-piperidyl]pyrimidin-4-yl]amino]-1-methyl-2-oxo-3-quinolyl]oxy]-N-methyl-acetamide as a yellow solid (65 mg, 29%).

### 2-((6-((2-((3S,5R)-3-(Aminomethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (50a)

To a solution of 2-[[6-[[5-chloro-2-[(3R,5R)-3-[(1,3-dioxoisoindolin-2-yl)methyl]-4,4-difluoro-5-methyl-1-piperidyl]pyrimidin-4-yl]amino]-1-methyl-2-oxo-3-quinolyl]oxy]-N-methyl-acetamide (70.00 mg, 105.09 μmol, 1 eq) in EtOH (2 mL) was added NH₂NH₂—H₂O (78.92 mg, 1.58 mmol, 76.62 μL, 15 eq). The reaction mixture was stirred at 60° C. for 2 hr. The reaction mixture was concentrated under reduced pressure and the residue was purified by prep-HPLC (column: Phenomenex Luna 80*30 mm*3 μm; mobile phase: [water (0.04% HCl)—MeCN]; B %: 15%-35%, 8 min) to give the title compound as a white solid (6.1 mg, 6%, HCl salt). ¹H NMR (400 MHz, MeOD) δ=8.03 (s, 1H), 7.81 (d, J=2.4 Hz, 1H), 7.68-7.60 (m, 1H), 7.54 (d, J=9.2 Hz, 1H), 7.29 (s, 1H), 4.57 (br d, J=13.8 Hz, 1H), 4.50 (s, 2H), 4.30 (br d, J=12.8 Hz, 1H), 3.73 (s, 3H), 3.28-3.22 (m, 1H), 3.03-2.79 (m, 3H), 2.77 (s, 3H), 2.43 (br d, J=13.2 Hz, 1H), 2.18-2.11 (m, 1H), 0.97 (d, J=6.8 Hz, 3H). LCMS: [M+1]⁺=536.2.

### 2-((6-((2-((3R,5S)-3-(aminomethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (50b)

To a solution of 2-[[6-[[5-chloro-2-[(3S,5S)-3-[(1,3-dioxoisoindolin-2-yl)methyl]-4,4-difluoro-5-methyl-1-piperidyl]pyrimidin-4-yl]amino]-1-methyl-2-oxo-3-quinolyl]oxy]-N-methyl-acetamide (65 mg, 97.59 μmol, 1 eq) in EtOH (2 mL) was added NH₂NH₂—H₂O (73.28 mg, 1.46 mmol, 71.14 μL, 15 eq). The reaction mixture was stirred at 60° C. for 2 hr. The reaction mixture was concentrated under reduced pressure and the residue was purified by prep-HPLC (column: Phenomenex Luna 80*30 mm*3 μm; mobile phase: [water (0.04% HCl)—MeCN], B %: 15%-35%, 8 min) to give the title compound as a white solid (13.7 mg, 24%, HCl salt). ¹H NMR (400 MHz, MeOD) δ=8.16 (s, 1H), 7.96 (d, J=2.4 Hz, 1H), 7.79-7.72 (m, 1H), 7.65 (d, J=9.2 Hz, 1H), 7.43 (s, 1H), 4.73 (br d, J=12.0 Hz, 1H), 4.61 (s, 2H), 4.41 (br d, J=13.6 Hz, 1H), 3.79 (s, 3H), 3.40-3.34 (m, 1H), 3.15-2.91 (m, 3H), 2.89 (s, 3H), 2.70-2.53 (m, 1H), 2.43-2.19 (m, 1H), 1.09 (d, J=6.8 Hz, 3H). LCMS: [M+1]⁺=536.2.

The absolute configurations of compounds 50a & 50b were randomly assigned based on the alkylamino group and methyl group being in cis-conformation.

### Example 4: Synthesis of 2-((6-((2-((3S,5R)-3-(2-aminoethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (51a) and 2-((6-((2-((3R,5S)-3-(2-aminoethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (51b)

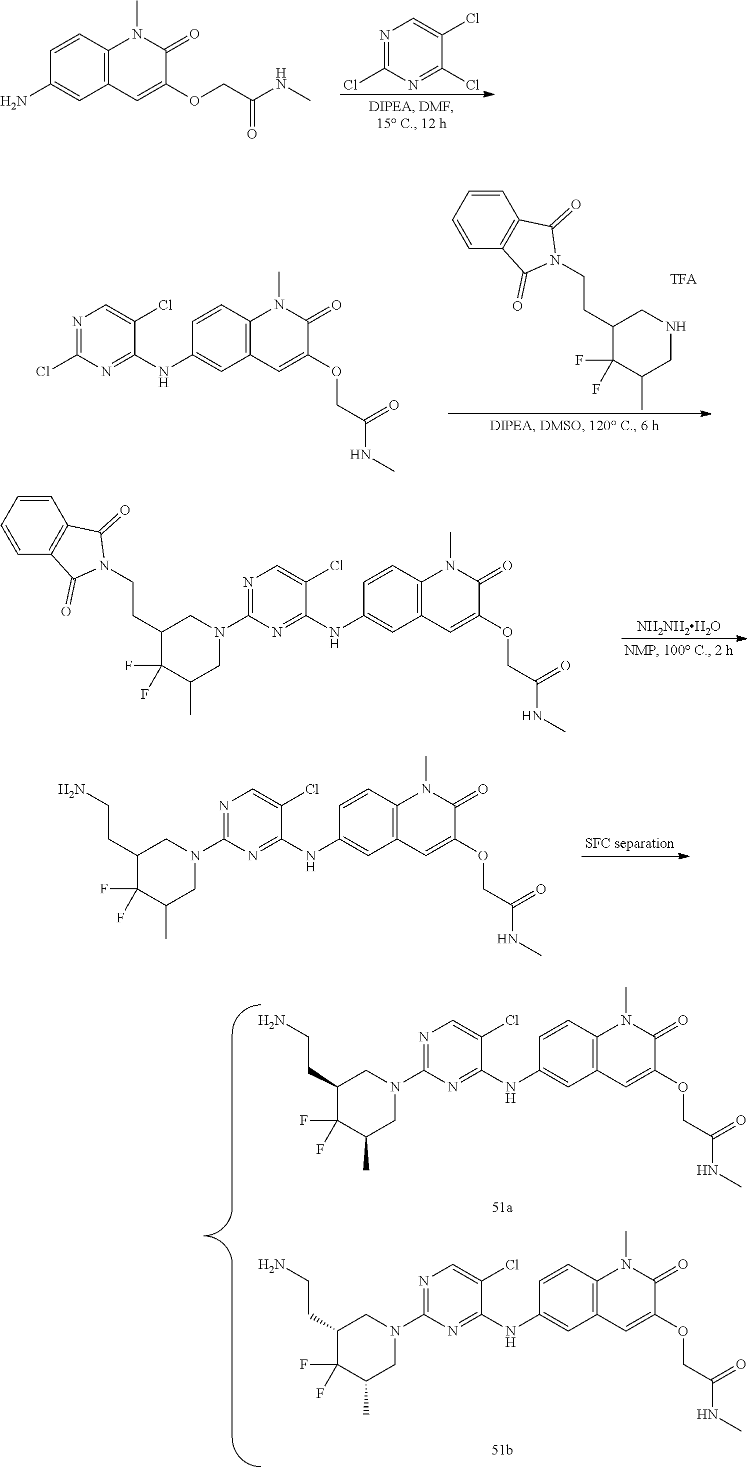

### 2-((6-((2,5-Dichloropyrimidin-4-yl)amino)-1-methyl-2-oxo-1,2-dihydroquinolin-3-yl)oxy)-N-methylacetamide

To a solution of 2-[(6-amino-1-methyl-2-oxo-3-quinolyl)oxy]-N-methyl-acetamide (3 g, 11.48 mmol, 1 eq) and 2,4,5-trichloropyrimidine (3.16 g, 17.22 mmol, 1.5 eq) in DMF (30 mL) was added DIEA (2.23 g, 17.22 mmol, 3.00 mL, 1.5 eq). The reaction mixture was stirred at 15° C. for 12 hr. Water (50 mL) was added and the precipitated solids were filtered and washed with H₂O (150 mL) and ethyl acetate (200 mL). The solid was dried under reduced pressure to give the title compound (3.98 g, 85%). ¹H NMR (400 MHz, DMSO-d₆) δ 9.65 (s, 1H), 8.38 (s, 1H), 7.94 (s, 1H), 7.75 (s, 1H), 7.66 (d, J=8.4 Hz, 1H), 7.53 (d, J=8.8 Hz, 1H), 7.23 (s, 1H), 4.58 (s, 2H), 3.69 (s, 3H), 2.67 (d, J=3.6 Hz, 3H).

### 2-((6-((5-Chloro-2-(3-(2-(1, 3-dioxoisoindolin-2-yl)ethyl)-4, 4-difluoro-5-methylpiperidin-1-yl)pyrimidin-4-yl)amino)-1-methyl-2-oxo-1,2-dihydroquinolin-3-yl)oxy)-N-methylacetamide

A mixture of 2-[[6-[(2,5-dichloropyrimidin-4-yl)amino]-1-methyl-2-oxo-3-quinolyl]oxy]-N-methyl-acetamide (600 mg, 1.47 mmol, 1 eq), 2-[2-(4,4-difluoro-5-methyl-3-piperidyl)ethyl]isoindoline-1,3-dione (931.10 mg, 2.20 mmol, 1.5 eq, TFA), and DIEA (379.90 mg, 2.94 mmol, 512.00 μL, 2 eq) in DMSO (10 mL) stirred at 120° C. for 6 hr under N₂ atmosphere. Water (15 mL) was added and the precipitated solids were filtered and washed with H₂O (30 mL) and ethyl acetate (30 mL). The solid was dried under reduced pressure to give the title compound as a white solid (1 g, crude). ¹H NMR (400 MHz, DMSO-d₆) δ 8.99 (s, 1H), 8.03 (s, 1H), 7.84 (s, 5H), 7.74 (d, J=6.8 Hz, 2H), 7.46 (d, J=8.8 Hz, 1H), 7.19 (s, 1H), 4.58-4.48 (m, 4H), 3.56 (s, 3H), 3.30 (s, 2H), 2.69-2.64 (m, 5H), 2.05-1.90 (m, 3H), 1.42 (s, 1H), 0.94 (d, J=6.8 Hz, 3H).

### 2-((6-((2-(3-(2-Aminoethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide

To a mixture of 2-[[6-[[5-chloro-2-[3-[2-(1,3-dioxoisoindolin-2-yl)ethyl]-4,4-difluoro-5-methyl-1-piperidyl]pyrimidin-4-yl]amino]-1-methyl-2-oxo-3-quinolyl]oxy]-N-methyl-acetamide (400 mg, 588.15 μmol, 1 eq) in NMP (5 mL) was added NH₂NH₂— H₂O (480.00 mg, 9.59 mmol, 466.02 μL, 16.30 eq) and the mixture was stirred at 100° C. for 2 hr. The reaction mixture was concentrated under reduced pressure to give a residue which was purified by prep-HPLC (column: Phenomenex Luna C18 (250*70 mm, 15 μm); mobile phase: [water (0.1% TFA)-MeCN]; B %: 12%-35%, 21 min) to give the title compound as a white solid (250 mg, 77%). ¹H NMR (400 MHz, MeOD) δ 8.14 (s, 1H), 7.87 (d, J=2.0 Hz, 1H), 7.76 (dd, J=9.2, 2.0 Hz, 1H), 7.62 (d, J=9.2 Hz, 1H), 7.31 (s, 1H), 4.57 (s, 2H), 4.48 (d, J=13.2 Hz, 1H), 4.38 (d, J=12.8 Hz, 1H), 3.83 (s, 3H), 3.06-2.96 (m, 3H), 2.85 (s, 3H), 2.21-2.06 (m, 3H), 2.03 (s, 1H), 1.68-1.58 (m, 1H), 1.03 (d, J=6.4 Hz, 3H).

### 2-((6-((2-((3S,5R)-3-(2-Aminoethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (51a) and 2-((6-((2-((3R,5S)-3-(2-aminoethyl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (51b)

2-[[6-[[2-[3-(2-aminoethyl)-4,4-difluoro-5-methyl-1-piperidyl]-5-chloro-pyrimidin-4-yl]amino]-1-methyl-2-oxo-3-quinolyl]oxy]-N-methyl-acetamide (150 mg, 225.90 μmol, TFA) was separated by SFC (column: DAICEL CHIRALPAK AD-H (250 mm*30 mm, 5 μm); mobile phase: [0.1% NH₃H₂O IPA]; B %: 30% -30%, 15 min) to give 51a and 51b each as white solids. 51a: (12 mg, 10% yield, 100% purity); ¹H NMR (400 MHz, MeOD) δ 7.99 (s, 1H), 7.87 (d, J=2.0 Hz, 1H), 7.80 (dd, J=9.2, 2.4 Hz, 3H), 7.57 (d, J=9.2 Hz, 1H), 7.32 (s, 1H), 4.69 (d, J=11.6 Hz, 1H), 4.59-4.55 (m, 3H), 3.85 (s, 3H), 2.87 (s, 3H), 2.77-2.66 (m, 4H), 2.04-1.89 (m, 3H), 1.44-1.38 (m, 1H), 1.02 (d, J=6.8 Hz, 3H); LCMS: [M+H]⁺=550.2. 51b: (4 mg, 3% yield, 95% purity); ¹H NMR (400 MHz, DMSO-d₆) δ 8.96 (s, 1H), 8.08 (s, 1H), 8.00 (s, 1H), 7.81 (s, 1H), 7.72 (d, J=9.6 Hz, 1H), 7.48 (d, J=8.8 Hz, 1H), 7.22 (s, 1H), 4.65-4.47 (m, 4H), 3.69 (s, 3H), 2.66-2.57 (m, 7H), 2.07-1.92 (m, 4H), 1.76-1.67 (m, 1H), 1.23 (s, 1H), 0.94 (d, J=4.4 Hz, 3H); LCMS: [M+H*]=550.1.

The absolute configurations of compounds 51a & 51b were randomly assigned based on the alkylamino group and methyl group being in cis-conformation.

### Example 5: Synthesis of 2-((6-((5-chloro-2-(3-(2-(dimethylamino)ethyl)-4,4-difluoro-5-methyl piperidin-1-yl)pyrimidin-4-yl)amino)-1-methyl-2-oxo-1,2-dihydroquinolin-3-yl)oxy)-N-methylacetamide (53)

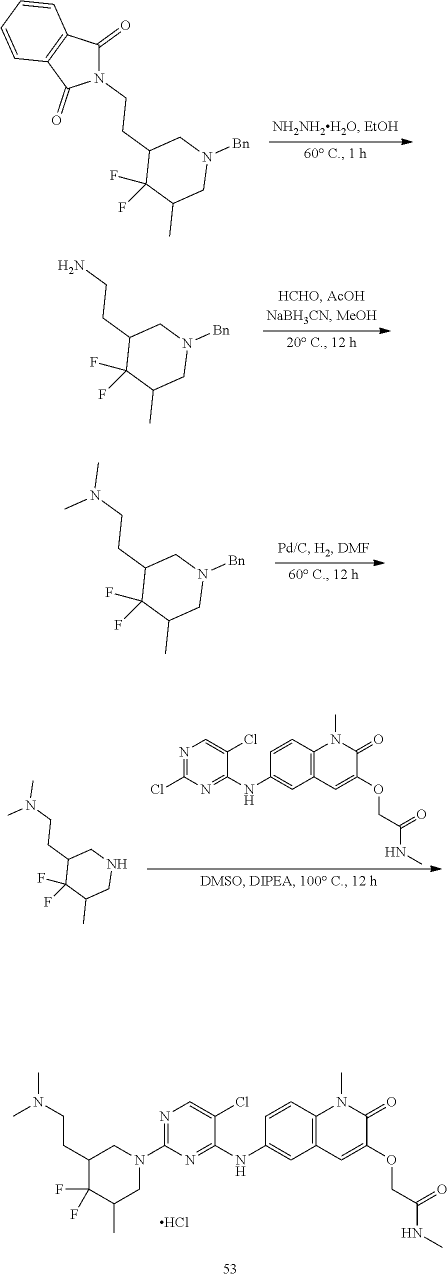

### 2-(1-Benzyl-4,4-difluoro-5-methylpiperidin-3-yl)ethan-1-amine

To a solution of 2-[2-(1-benzyl-4,4-difluoro-5-methyl-3-piperidyl)ethyl]isoindoline-1,3-dione (2.6 g, 6.53 mmol, 1 eq) in EtOH (30 mL) was added NH₂NH₂—H₂O (3.27 g, 65.25 mmol, 3.17 mL, 10 eq) and the reaction mixture was stirred at 60° C. for 1 h. The reaction mixture was cooled to 20° C. and filtered. The filtrate was concentrated under reduced pressure to give the title compound as a colorless oil (1.15 g, 64% yield, 98% purity). LCMS: [M+H]⁺=269.0.

### 2-(1-Benzyl-4,4-difluoro-5-methylpiperidin-3-yl)-N,N-dimethylethan-1-amine

To a solution of 2-(1-benzyl-4,4-difluoro-5-methyl-3-piperidyl)ethanamine (1 g, 3.73 mmol, 1 eq) in MeOH (15 mL) was added HCHO aq. (2.42 g, 29.81 mmol, 2.22 mL, 37% purity, 8 eq) and AcOH (111.89 mg, 1.86 mmol, 106.57 μL, 0.5 eq). The reaction mixture was stirred at 20° C. for 30 min and then NaBH₃CN (1.87 g, 29.81 mmol, 8 eq) was added and the reaction mixture was stirred at 20° C. for 11.5 h. The reaction mixture was quenched by adding water (50 mL) and extracted with ethyl acetate (30 mL×2). The combined organic phase was washed with brine (50 mL), dried with anhydrous Na₂SO₄, filtered and the filtrate was concentrated under reduced pressure. The residue was purified by p-HPLC (column: Phenomenex Luna C18 (250*70 mm, 15 μm); mobile phase: [water(HCl)-ACN]; B %: 1%-20%,20 min) to give the title compound as a colorless oil (320 mg, 29% yield, 100% purity). ¹H NMR (400 MHz, MeOD) δ=7.38-7.27 (m, 5H), 3.65-3.56 (m, 2H), 3.23-3.18 (m, 2H) 2.86 (s, 7H), 2.22-2.01 (m, 6H) 1.64-1.61 (m, 1H) 0.97 (d, J=6.63 Hz, 3H).

### 2-(4,4-Difluoro-5-methylpiperidin-3-yl)-N,N-dimethylethan-1-amine

To a solution of 2-(1-benzyl-4,4-difluoro-5-methyl-3-piperidyl)-N,N-dimethyl-ethanamine (160 mg, 539.82 μmol, 1 eq) in DMF (3 mL) was added Pd/C (0.1 g, 67.48 μmol, 10% purity) under Ar. The suspension was degassed under vacuum and purged with H₂ several times.

The reaction mixture was stirred under H₂ (15 psi) at 60° C. for 12 hours. The reaction mixture was filtered, and the filtrate was concentrated under reduced pressure to give the title compound as a yellow oil (100 mg, 90% yield). ¹H NMR (400 MHz, DMSO-d₆) δ ppm 3.57-3.49 (m, 1H), 2.37-2.31 (m, 2H), 2.06-2.05 (m, 6H), 1.72-1.69 (m, 2H), 1.41-1.48 (m, 1H), 1.02-0.92 (m, 4H), 0.21 (dd, J=6.50, 1.75 Hz, 3H).

### 2-((6-((5-Chloro-2-(3-(2-(dimethylamino)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide (53)

A mixture of 2-[[6-[(2,5-dichloropyrimidin-4-yl)amino]-1-methyl-2-oxo-3-quinolyl]oxy]-N-methyl-acetamide (100 mg, 244.96 μmol, 1 eq), 2-(4,4-difluoro-5-methyl-3-piperidyl)-N,N-dimethyl-ethanamine (100 mg, 484.79 μmol, 1.98 eq) and DIPEA (63.32 mg, 489.91 μmol, 85.33 μL, 2 eq) in DMSO (2 mL) was stirred at 100° C. for 12 h. The mixture was purified directly by p-HPLC (column: Phenomenex Luna 80*30 mm*3 um; mobile phase: [water(HCl)-ACN]; B %: 1%-30%, 8 min) to give the title compound as a white solid (12 mg, 8% yield, 94% purity, HCl salt). ¹H NMR (400 MHz, DMSO-d₆) δ=10.43 (br s, 1H), 9.44 (br s, 1H), 8.18-8.11 (m, 1H), 8.02 (br d, J=3.8 Hz, 1H), 7.82 (br s, 1H), 7.73 (br d, J=7.9 Hz, 1H), 7.55 (br d, J=8.9 Hz, 1H), 7.24 (s, 1H), 4.58 (s, 4H), 3.69 (br s, 3H), 3.18-3.01 (m, 3H), 2.85-2.74 (m, 2H), 2.73-2.65 (m, 8H), 2.04 (br s, 3H), 1.73-1.58 (m, 1H), 0.94 (br d, J=6.6 Hz, 3H). LCMS: [M+H]⁺=578.2.

### Example 6: Synthesis of 2-((6-((2-(3-(1-amino-2-methylpropan-2-yl)-4,4-difluoro-5-methyl piperidin-1-yl)-5-chloropyrimidin-4-yl)amino)-1-methyl-2-oxo-1,2-dihydroquinolin-3-yl)oxy)-N-methylacetamide (57)

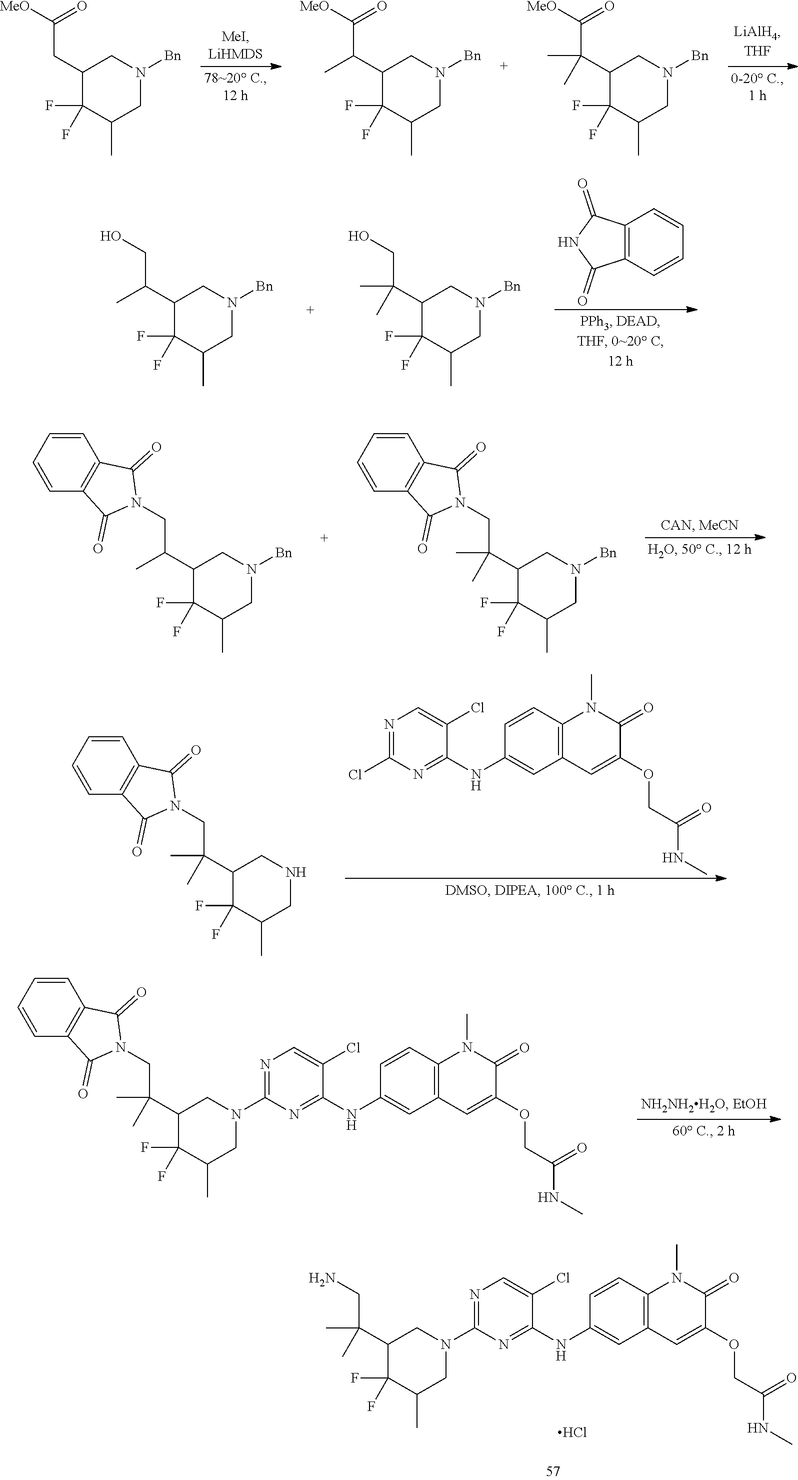

### Methyl 2-(1-benzyl-4,4-difluoro-5-methylpiperidin-3-yl)propanoate and methyl 2-(1-benzyl-4,4-difluoro-5-methylpiperidin-3-yl)-2-methylpropanoate

To a solution of methyl 2-(1-benzyl-4,4-difluoro-5-methyl-3-piperidyl)acetate (2.00 g, 6.73 mmol, 1 eq) in THE (30 mL) was added LiHMDS (1 M, 7.40 mL, 1.1 eq) dropwise at −78° C. under N₂. After addition, the reaction mixture was stirred at −78° C. for 15 min, and then Mel (1.05 g, 7.40 mmol, 460.53 μL, 1.1 eq) was added dropwise at −78° C. The resulting mixture was warmed to 20° C. over 1 hr. The reaction mixture was cooled to −78° C. and LiHMDS (1 M, 7.40 mL, 1.1 eq) was added dropwise at −78° C. under N₂. After addition, the reaction mixture was stirred at −78° C. for 15 min, and then Mel (1.05 g, 7.40 mmol, 460.61 μL, 1.1 eq) was added dropwise at −78° C. under N₂. The resulting mixture was warmed to 20° C. over 1 h and then stirred at 20° C. for 9.5 h. The reaction mixture was cooled to 0° C. and quenched with sat. aq. NH₄Cl (100 mL) and extracted with ethyl acetate (80 mL×2). The combined organic phase was washed with brine (100 mL), dried with anhydrous Na₂SO₄, filtered and the filtrate was concentrated under reduced pressure. The residue was purified by silica gel chromatography (silica gel, Petroleum ether/Ethyl acetate=10/1, 2/1) to give a mixture of the title compounds as a yellow oil (2.2 g). LCMS: [M+H]+=326.1.

### 2-(1-Benzyl-4,4-difluoro-5-methylpiperidin-3-yl)propan-1-ol and 2-(1-benzyl-4,4-difluoro-5-methylpiperidin-3-yl)-2-methylpropan-1-ol

To a mixture of 2 g of methyl 2-(1-benzyl-4,4-difluoro-5-methyl-3-piperidyl)-2-methyl-propanoate (6.15 mmol, 1 eq) and methyl 2-(1-benzyl-4,4-difluoro-5-methyl-3-piperidyl)propanoate (6.15 mmol, 1 eq) in THF (20 mL) was added LAH (279.91 mg, 7.38 mmol, 1.2 eq) at 0° C. After addition, the reaction mixture was stirred at 20° C. for 1 hr and then the reaction mixture was cooled to 0° C. and quenched with water (2 mL). 10 g of anhydrous Na₂SO₄ was added and the mixture was stirred for 2 min. The mixture was filtered, and the filtrate was concentrated under reduced pressure to give a mixture of the title compounds as a yellow oil (1.6 g). LCMS: [M+H]⁺=298.1.

### 2-(2-(1-Benzyl-4,4-difluoro-5-methylpiperidin-3-yl)propyl)isoindoline-1,3-dione and 2-(2-(1-benzyl-4,4-difluoro-5-methylpiperidin-3-yl)-2-methylpropyl)isoindoline-1,3-dione

To a solution of 1.1 g of 2-(1-benzyl-4,4-difluoro-5-methyl-3-piperidyl)-2-methyl-propan-1-ol (1.68 mmol, 1 eq) and 2-(1-benzyl-4,4-difluoro-5-methyl-3-piperidyl) propan-1-ol (1.68 mmol, 1 eq), isoindoline-1,3-dione (321.59 mg, 2.19 mmol, 1.3 eq) and PPh₃ (573.30 mg, 2.19 mmol, 1.3 eq) in THF (20 mL) was added DEAD (380.67 mg, 2.19 mmol, 397.36 μL, 1.3 eq) at 0° C. under N₂. The reaction mixture was slowly warmed to 20° C. and stirred at 20° C. for 12 h. The reaction mixture (combined with another batch with 0.5 g scale) was quenched with water (50 mL) and extracted with ethyl acetate (50 mL×2). The combined organic phase was washed with brine (50 mL), dried with anhydrous Na₂SO₄, filtered and the filtrate was concentrated under reduced pressure. The residue was purified by reversed-phase MPLC (neutral condition) to give 2-[2-(1-benzyl-4,4-difluoro-5-methyl-3-piperidyl)-2-methyl-propyl]isoindoline-1,3-dione as a yellow oil (400 mg, 73.3% purity, LCMS: [M+H]⁺=427.1) and 2-[2-(1-benzyl-4,4-difluoro-5-methyl-3-piperidyl)propyl]isoindoline-1,3-dione as a yellow oil (600 mg, 89.4% purity, LCMS: [M+H]⁺=413.1.

### 2-(2-(4,4-Difluoro-5-methylpiperidin-3-yl)-2-methylpropyl)isoindoline-1,3-dione

To a solution of 2-[2-(1-benzyl-4,4-difluoro-5-methyl-3-piperidyl)-2-methyl-propyl]isoindoline-1,3-dione (300 mg, 703.40 μmol, 1 eq) in ACN (12 mL) and H₂O (3 mL) was added CAN (3.86 g, 7.03 mmol, 3.51 mL, 10 eq) and was stirred at 50° C. for 12 h. To the reaction mixture (combined with another batch at 100 mg scale) was added sat. aq. Na₂CO₃ (˜10 mL). The reaction mixture was filtered and the filter cake was washed by water (15 mL×2) and EtOAc (15 mL×2).

The filtrate was extracted with ethyl acetate (30 mL×3). The combined organic phase was dried with anhydrous Na₂SO₄, filtered and the filtrate was concentrated under reduced pressure. The residue was purified by flash silica gel chromatography (Silica Flash Column, Eluent of 0˜30% MeOH/DCM) to give the title compound as a yellow solid (130 mg). LCMS: [M+H]⁺=337.1.

### 2-((6-((5-Chloro-2-(3-(1-(1,3-dioxoisoindolin-2-yl)-2-methylpropan-2-yl)-4,4-difluoro-5-methylpiperidin-1-yl)pyrimidin-4-yl)amino)-1-methyl-2-oxo-1,2-dihydroquinolin-3-yl)oxy)-N-methylacetamide

To a solution of 2-[2-(4,4-difluoro-5-methyl-3-piperidyl)-2-methyl-propyl]isoindoline-1,3-dione (130 mg, 386.47 μmol, 1 eq) and 2-[[6-[(2,5-dichloropyrimidin-4-yl)amino]-1-methyl-2-oxo-3-quinolyl]oxy]-N-methyl-acetamide (157.77 mg, 386.47 μmol, 1 eq) in DMSO (2 mL) was added DIPEA (99.90 mg, 772.95 μmol, 134.63 μL, 2 eq) and the reaction mixture was stirred at 100° C. for 1 hr. The reaction mixture was cooled to 20° C. and water (2 mL) was added. The mixture was filtered and the filter cake was washed by water (5 mL×2) and EtOAc (10 mL×2). The filter cake was dried to give the title compound as a white solid (40 mg, 13% yield, 90% purity). ¹H NMR (400 MHz, DMSO-d₆) δ=8.97 (s, 1H), 8.09 (s, 1H), 7.82-7.75 (m, 5H), 7.65-7.52 (m, 2H), 7.34 (s, 1H), 7.11 (s, 1H), 4.73-4.34 (m, 4H), 3.74-3.39 (m, 3H), 3.28-2.87 (m, 3H), 2.72-2.61 (m, 4H), 2.11-1.64 (m, 2H), 0.93 (d, J=6.4 Hz, 3H), 0.77 (s, 6H).

### 2-((6-((2-(3-(1-Amino-2-methylpropan-2-yl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (57)

To a solution of 2-[[6-[[5-chloro-2-[3-[2-(1,3-dioxoisoindolin-2-yl)-1,1-dimethyl-ethyl]-4,4-difluoro-5-methyl-1-piperidyl]pyrimidin-4-yl]amino]-1-methyl-2-oxo-3-quinolyl]oxy]-N-methyl-acetamide (40 mg, 56.49 μmol, 1 eq) in EtOH (1 mL) was added NH₂NH₂·H₂O (42.41 mg, 847.28 μmol, 41.18 μL, 15 eq) and the reaction mixture was stirred at 60° C. for 2 h. The reaction mixture was concentrated under reduced pressure and purified directly by prep-HPLC (column: Phenomenex Luna 80*30 mm*3 m; mobile phase: [water(HCl)-ACN]; B %: 20%-40%, 8 min) to give the title compound as a white solid (5.8 mg, 14% yield, 99% purity, HCl). ¹H NMR (400 MHz, DMSO-d₆) δ=9.14 (s, 1H), 8.12 (s, 1H), 7.99 (d, J=4.4 Hz, 1H), 7.78 (s, 1H), 7.78 (br s, 2H), 7.68 (dd, J=2.4, 9.2 Hz, 1H), 7.47 (d, J=9.2 Hz, 1H), 7.26 (s, 1H), 4.69-4.43 (m, 4H), 3.68 (s, 3H), 2.94 (br t, J=10.8 Hz, 2H), 2.74-2.66 (m, 5H), 2.03-1.92 (m, 2H), 1.07-0.71 (m, 9H). LCMS: [M+H]⁺=578.2.

### Example 7: Synthesis of 2-((6-((2-(3-(1-aminopropan-2-yl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (56)

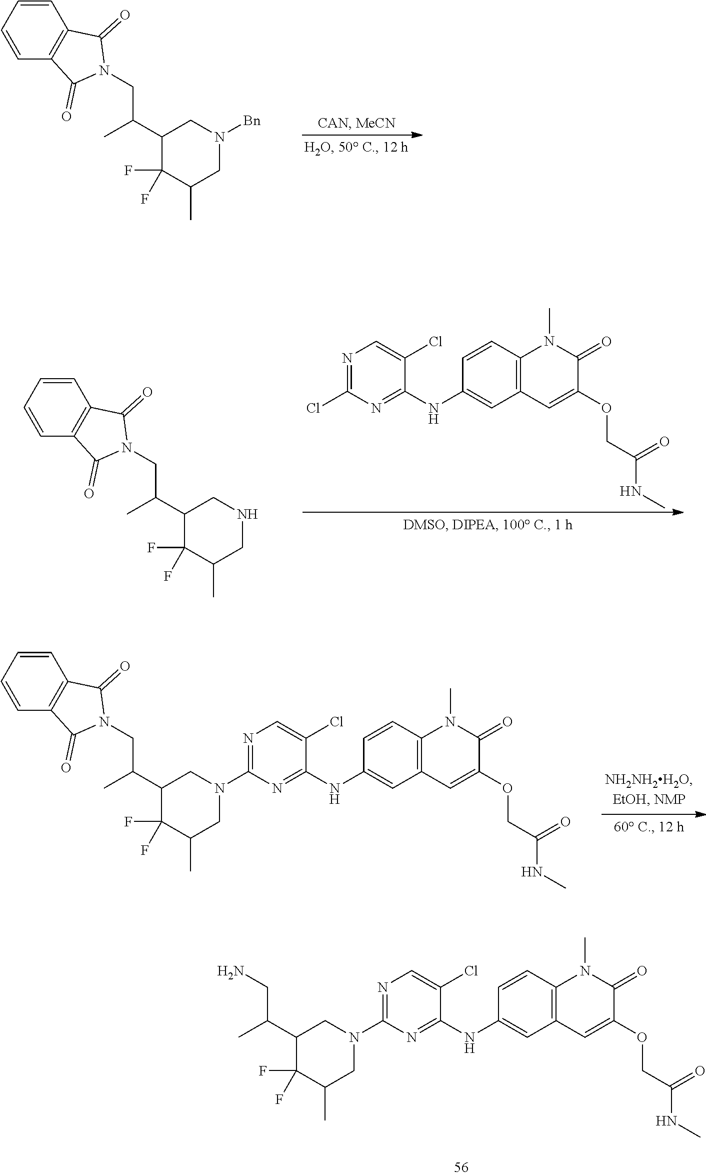 [SMILES: CC(CN1C(=O)c2ccccc2C1=O)C1CN(Cc2ccccc2)CC(C)C1(F)F ; CC(CN1C(=O)c2ccccc2C1=O)C1CNCC(C)C1(F)F ; CNC(=O)COc1cc2cc(Nc3nc(Cl)ncc3Cl)ccc2n(C)c1=O ; CNC(=O)COc1cc2cc(Nc3nc(N4CC(C)C(F)(F)C(C(C)CN5C(=O)c6ccccc6C5=O)C4)ncc3Cl)ccc2n(C)c1=O ; CNC(=O)COc1cc2cc(Nc3nc(N4CC(C)C(F)(F)C(C(C)CN)C4)ncc3Cl)ccc2n(C)c1=O]

### 2-(2-(4,4-Difluoro-5-methylpiperidin-3-yl)propyl)isoindoline-1, 3-dione

To a solution of 2-[2-(1-benzyl-4,4-difluoro-5-methyl-3-piperidyl)propyl]isoindoline-1, 3-dione (500 mg, 1.21 mmol, 1 eq) in MeCN (20 mL) and H₂O (5 mL) was added CAN (6.65 g, 12.12 mmol, 6.04 mL, 10 eq). The reaction mixture was stirred at 50° C. for 12 hr. The pH of the reaction mixture (combined with another batch with 100 mg scale) was adjusted to 9-10 by adding sat. aq. Na₂CO₃ and then the reaction mixture was filtered and washed with EtOAc (15 mL×2).

Water (20 mL) was added to the filtrate and extracted with ethyl acetate (20 mL×3). The combined organic phase was dried with anhydrous Na₂SO₄, filtered and the filtrate was concentrated under reduced pressure. The residue was purified by flash silica gel chromatography (Silica Flash Column, Eluent of 0˜50% Ethylacetate/MeOH) to give the title compound as a yellow solid (250 mg, 73% purity). LCMS: [M+H]⁺=323.0.

### 2-((6-((5-Chloro-2-(3-(1-(1,3-dioxoisoindolin-2-yl)propan-2-yl)-4,4-difluoro-5-methylpiperidin-1-yl)pyrimidin-4-yl)amino)-1-methyl-2-oxo-1,2-dihydroquinolin-3-yl)oxy)-N-methylacetamide

To a solution of 2-[2-(4,4-difluoro-5-methyl-3-piperidyl)propyl]isoindoline-1,3-dione (200 mg, 620.45 μmol, 1 eq) and 2-[[6-[(2,5-dichloropyrimidin-4-yl)amino]-1-methyl-2-oxo-3-quinolyl]oxy]-N-methyl-acetamide (253.29 mg, 620.45 mol, 1 eq) in DMSO (2 mL) was added DIPEA (160.38 mg, 1.24 mmol, 216.14 μL, 2 eq) and the reaction mixture was stirred at 100° C. for 1 h. Water (5 mL) was added to the reaction mixture and filtered. The filter cake was washed with water (15 mL×2) and EtOAc (15 mL×2). The solid was dried under reduced pressure to give the title compound as a white solid (200 mg, 98% purity) as a white solid. ¹H NMR (400 MHz, DMSO-d₆) δ=9.04-8.92 (m, 1H), 8.12-8.02 (m, 1H), 7.99-7.75 (m, 5H), 7.73 (d, J=8.0 Hz, 1H), 7.45 (d, J=9.2 Hz, 1H), 7.16 (d, J=21.6 Hz, 1H), 4.74-4.29 (m, 4H), 3.81-3.40 (m, 4H), 3.31-3.00 (m, 2H), 2.91-2.84 (m, 1H), 2.73-2.64 (m, 4H), 2.54 (s, 1H), 2.13-1.80 (m, 2H), 1.07-0.67 (m, 6H). LCMS: [M+H]⁺=694.2. [00320]2-((6-((2-(3-(1-Aminopropan-2-yl)-4,4-difluoro-5-methylpiperidin-1-yl)-5-chloropyrimidin-4-yl)amino)-1-methyl-2-oxo-1,2-dihydroquinolin-3-yl)oxy)-N-methylacetamide (56)

To a solution of 2-[[6-[[5-chloro-2-[3-[2-(1,3-dioxoisoindolin-2-yl)-1-methyl-ethyl]-4,4-difluoro-5-methyl-1-piperidyl]pyrimidin-4-yl]amino]-1-methyl-2-oxo-3-quinolyl]oxy]-N-methyl-acetamide (150 mg, 216.10 μmol, 1 eq) in EtOH (2 mL) and NMP (2 mL) added NH₂NH₂·H₂O (162.27 mg, 3.24 mmol, 157.55 μL, 15 eq) and the reaction mixture was stirred at 60° C. for 12 h. Water (5 mL) was added to the reaction mixture and filtered. The filter cake was washed with water (15 mL×2), EtOAc (15 mL×2), and EtOH (15 mL×2). The solid was dried under reduced pressure to give the title compound as a white solid (75.4 mg, ⁴⁶.% yield, 92% purity). ¹H NMR (400 MHz, DMSO-d₆) δ=8.95 (s, 1H), 8.07 (m, 1H), 8.02-7.89 (m, 1H), 7.82 (br d, J=5.6 Hz, 1H), 7.71 (dd, J=2, 9.2 Hz, 1H), 7.47 (d, J=9.2 Hz, 1H), 7.30-7.11 (m, 1H), 4.63-4.42 (m, 4H), 3.68 (s, 3H), 2.83-2.73 (m, 1H), 2.69-2.61 (m, 5H), 2.04-1.70 (m, 2H), 0.95-0.83 (m, 6H). LCMS: [M+H]⁺=564.2.

### Example 8: Synthesis of 2-((6-((5-chloro-2-((3S,5R)-3-(2-(3,3-difluoroazetidin-1-yl)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide (54a) and 2-((6-((5-chloro-2-((3R,5S)-3-(2-(3,3-difluoroazetidin-1-yl)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide (54b)

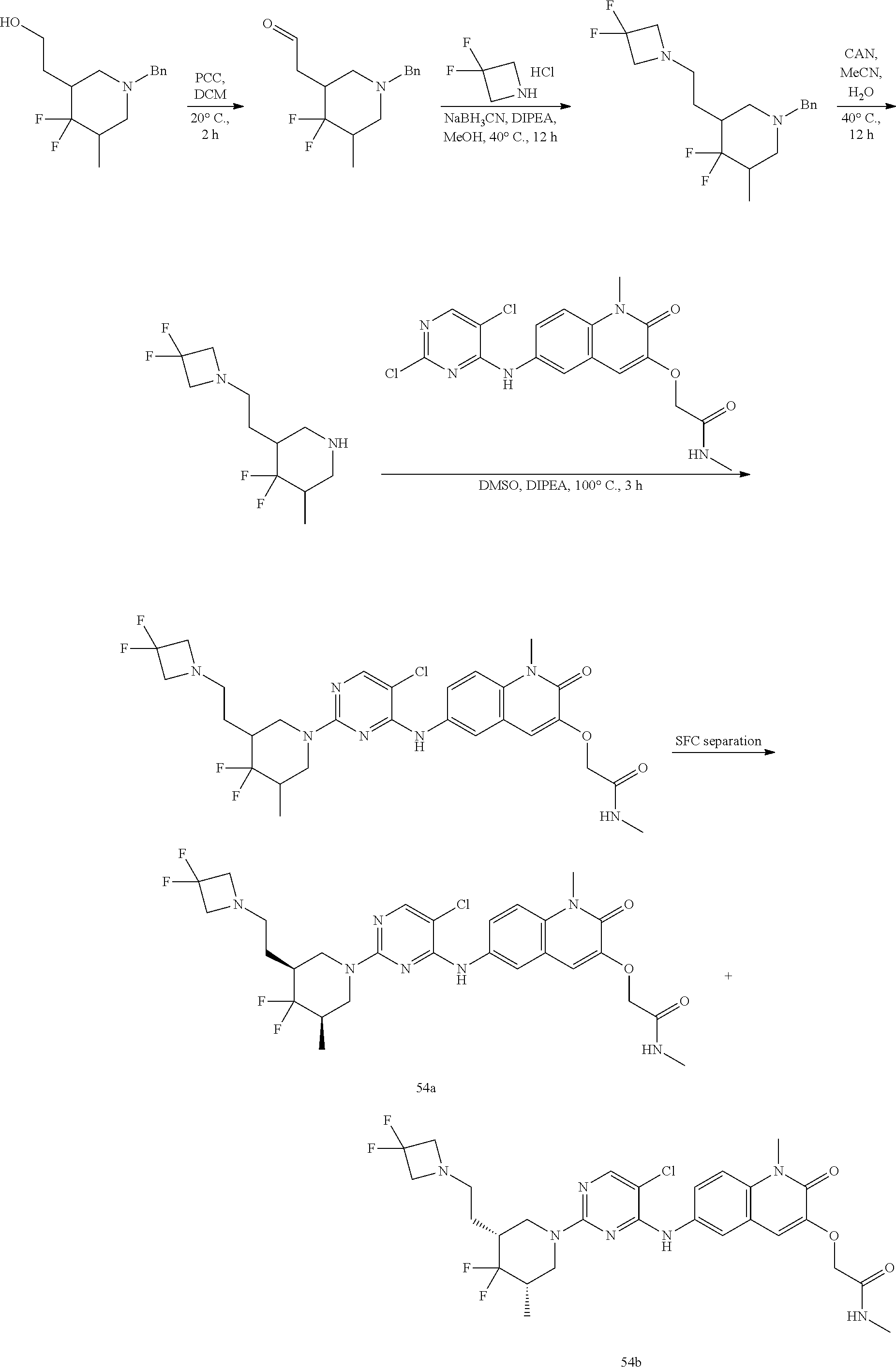

### 2-(1-Benzyl-4,4-difluoro-5-methylpiperidin-3-yl)acetaldehyde

To a solution of 2-(1-benzyl-4,4-difluoro-5-methyl-3-piperidyl)ethanol (3 g, 11.14 mmol, 1 eq) in DCM (30 mL) was added PCC (4.80 g, 22.28 mmol, 2 eq) and the reaction mixture was stirred at 20° C. for 1 h. The reaction mixture was filtered, and the filtrate was concentrated under reduced pressure. The residue was purified by flash silica gel chromatography (Silica Flash Column, Eluent of 10˜50% Ethyl acetate/Petroleum) to give the title compound as a yellow oil (1 g, 34% yield). LCMS: [M+H]⁺=268.0.

### 1-Benzyl-3-(2-(3,3-difluoroazetidin-1-yl)ethyl)-4,4-difluoro-5-methylpiperidine

To a solution of 2-(1-benzyl-4,4-difluoro-5-methyl-3-piperidyl)acetaldehyde (1 g, 3.74 mmol, 1 eq) and 3,3-difluoroazetidine (969.17 mg, 7.48 mmol, 2 eq, HCl) in MeOH (10 mL) was added DIPEA (1.93 g, 14.96 mmol, 2.61 mL, 4 eq) and the reaction mixture was stirred at 20° C. for 1 h. NaBH₃CN (470.17 mg, 7.48 mmol, 2 eq) was then added and the reaction mixture was stirred at 40° C. for 11 h. Sat. NaHCO₃ (40 mL) was added and the mixture was extracted with ethyl acetate (30 mL×3). The combined organic phase was washed with brine (50 mL), dried with anhydrous Na₂SO₄, filtered and concentrated under reduced pressure. The residue was purified by flash silica gel chromatography (Silica Flash Column, Eluent of 10˜50% Ethyl acetate/Petroleum ether gradient) to give the title compound as a yellow oil (300 mg, 22% yield, 96% purity). ¹H NMR (400 MHz, MeOD) δ=7.33-7.25 (m, 5H), 3.59-3.51 (m, 6H), 2.96-2.93 (m, 1H), 2.81-2.77 (m, 1H), 2.65-2.53 (m, 2H), 2.19-2.02 (m, 2H), 2.00-1.93 (m, 2H), 1.83-1.74 (m, 1H), 1.29-1.18 (m, 1H), 0.94 (d, J=6.8 Hz, 3H). LCMS: [M+H]⁺=345.1.

### 3-(2-(3,3-Difluoroazetidin-1-yl)ethyl)-4,4-difluoro-5-methylpiperidine

A mixture of 1-benzyl-3-[2-(3,3-difluoroazetidin-1-yl)ethyl]-4,4-difluoro-5-methyl-piperidine (200 mg, 580.74 mol, 1 eq), CAN (3.18 g, 5.81 mmol, 2.89 mL, 10 eq) in MeCN (8 mL) and H₂O (2 mL) was stirred at 40° C. for 12 h. The pH of the reaction mixture (combined with another batch with 100 mg scale) was adjusted to 10 by adding sat. aq Na₂CO₃ (20 mL). The reaction mixture was concentrated under reduced pressure and the concentrate was washed with THF (80 mL×2) and filtered. The filtrate was concentrated under reduced pressure to give the title compound as a yellow oil (230 mg, crude). LCMS: [M+H]⁺=255.2.

### 2-((6-((5-Chloro-2-(3-(2-(3,3-difluoroazetidin-1-yl)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide

A mixture of 2-[[6-[(2,5-dichloropyrimidin-4-yl)amino]-1-methyl-2-oxo-3-quinolyl]oxy]-N-methyl-acetamide (50 mg, 122.48 μmol, 1 eq), 3-[2-(3,3-difluoroazetidin-1-yl)ethyl]-4,4-difluoro-5-methyl-piperidine (152.66 mg, 306.19 μmol, 51% purity, 2.5 eq), DIPEA (31.66 mg, 244.96 μmol, 42.67 μL, 2 eq) in DMSO (1 mL) was stirred at 100° C. for 3 hr. The mixture (combined with another batch with 20 mg scale) was purified directly by p-HPLC (column: Phenomenex C18 80*40 mm*3 μm; mobile phase: [water(NH₄HCO₃)-ACN]; B %: 50%-80%, 8 min) to give the title compound as a white solid (50 mg, 100% purity). ¹H NMR (400 MHz, DMSO-d₆) δ=8.99 (s, 1H), 8.08 (s, 1H), 7.95 (br d, J=4.0 Hz, 1H), 7.85 (br s, 1H), 7.69 (dd, J=2.0, 8.8 Hz, 1H), 7.47 (d, J=9.2 Hz, 1H), 7.23 (s, 1H), 4.68-4.44 (m, 4H), 3.68 (s, 3H), 2.67-2.62 (m, 5H), 2.54-2.52 (m, 4H), 2.39-2.18 (m, 2H), 2.05-1.86 (m, 2H), 1.70-1.62 (m, 1H), 1.23-1.15 (m, 1H), 0.94 (br d, J=6.4 Hz, 3H).

### 2-((6-((5-chloro-2-((3S,5R)-3-(2-(3,3-difluoroazetidin-1-yl)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide (54a) and 2-((6-((5-chloro-2-((3R,5S)-3-(2-(3,3-difluoroazetidin-1-yl)ethyl)-4,4-difluoro-5-methylpiperidin-1-yl)pyrimidin-4-yl)amino)-1-methyl-2-oxo-1,2-dihydroquinolin-3-yl)oxy)-N-methylacetamide (54b)

2-[[6-[[5-chloro-2-[3-[2-(3,3-difluoroazetidin-1-yl)ethyl]-4,4-difluoro-5-methyl-1-piperidyl]pyrimidin-4-yl]amino]-1-methyl-2-oxo-3-quinolyl]oxy]-N-methyl-acetamide (50 mg, 79.87 μmol) was separated by SFC (column: DAICEL CHIRALPAK IG (250 mm*30 mm, 10 μm); mobile phase: [0.1% NH₃H₂O ETOH]; B %: 50%-50%, 10 min) to give compound 54a as a white solid (13.9 mg, 27% yield, 99% purity); ¹H NMR (400 MHz, DMSO-d₆) δ=8.99 (s, 1H), 8.08 (s, 1H), 7.96 (br d, J=4.4 Hz, 1H), 7.85 (s, 1H), 7.69 (dd, J=2.0, 8.8 Hz, 1H), 7.47 (d, J=9.2 Hz, 1H), 7.23 (s, 1H), 4.59-4.52 (m, 4H), 3.68 (s, 3H), 2.67-2.62 (m, 5H), 2.52 (m, 4H), 2.38-2.32 (m, 2H), 2.04-1.86 (m, 2H), 1.70-1.62 (m, 1H), 1.23-1.15 (m, 1H), 0.94 (d, J=6.4 Hz, 3H); LCMS: [M+H]⁺=626.1; and compound 54b as a white solid (12.2 mg, 24% yield, 99% purity); ¹H NMR (400 MHz, DMSO-d₆) δ=8.99 (s, 1H), 8.08 (s, 1H), 7.96 (br d, J=4.4 Hz, 1H), 7.85 (s, 1H), 7.69 (dd, J=2.0, 8.8 Hz, 1H), 7.47 (d, J=9.2 Hz, 1H), 7.23 (s, 1H), 4.69-4.42 (m, 4H), 3.68 (s, 3H), 2.68-2.62 (m, 5H), 2.54-2.52 (m, 4H), 2.35-2.17 (m, 2H), 2.04-1.86 (m, 2H), 1.70-1.62 (m, 1H), 1.23-1.15 (m, 1H), 0.94 (d, J=6.4 Hz, 3H); LCMS: [M+H]⁺=626.1.

### Example 9: 5-[[2-[(3S,5R)-3-amino-4,4-difluoro-5-methyl-1-piperidyl]-5-chloro-pyrimidin-4-yl]amino]-3-(3-hydroxy-3-methyl-butyl)-1-methyl-benzimidazol-2-one (1)

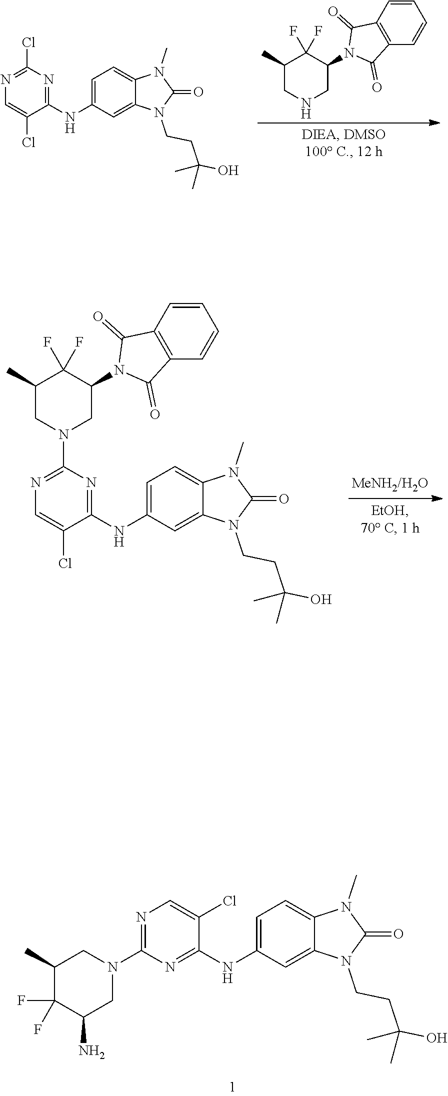 [SMILES: Cn1c(=O)n(CCC(C)(C)O)c2cc(Nc3nc(Cl)ncc3Cl)ccc21 ; C[C@@H]1CNC[C@H](N2C(=O)c3ccccc3C2=O)C1(F)F ; C[C@@H]1CN(c2ncc(Cl)c(Nc3ccc4c(c3)n(CCC(C)(C)O)c(=O)n4C)n2)C[C@H](N2C(=O)c3ccccc3C2=O)C1(F)F ; C[C@H]1CN(c2ncc(Cl)c(Nc3ccc4c(c3)n(CCC(C)(C)O)c(=O)n4C)n2)C[C@@H](N)C1(F)F]

### 2-[(3S,5R)-1-[5-Chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]isoindoline-1,3-dione

To a solution of 5-[(2,5-dichloropyrimidin-4-yl)amino]-3-(3-hydroxy-3-methyl-butyl)-1-methyl-benzimidazol-2-one (100 mg, 252.35 mol, 1 eq) in DMSO (1 mL) was added DIEA (65.22 mg, 504.7 μmol, 87.92 μL, 2 eq) and 2-[(3S,5R)-4,4-difluoro-5-methyl-3-piperidyl]isoindoline-1,3-dione (70.73 mg, 252.35 μmol, 1 eq). The reaction mixture was stirred at 100° C. for 12 hr under N₂. The reaction mixture was diluted with water (10 mL), filtered and the filter cake was dried under reduced pressure to give the title compound as a yellow solid (120 mg, 59% yield, 79% purity). LCMS: [M+H]⁺=640.3.

### 5-[[2-[(3S,5R)-3-Amino-4,4-difluoro-5-methyl-1-piperidyl]-5-chloro-pyrimidin-4-yl]amino]-3-(3-hydroxy-3-methyl-butyl)-1-methyl-benzimidazol-2-one (1)

To a solution of 2-[(3S,5R)-1-[5-chloro-4-[[3-(3-hydroxy-3-methyl-butyl)-1-methyl-2-oxo-benzimidazol-5-yl]amino]pyrimidin-2-yl]-4,4-difluoro-5-methyl-3-piperidyl]isoindoline-1,3-dione (120 mg, 187.48 μmol, 1 eq) in EtOH (6 mL) was added MeNH₂ (6 mL, 40% purity, in water) and the reaction mixture was stirred at 70° C. for 1 hr. The reaction mixture was concentrated under reduced pressure to give a residue. The residue was purified by prep-HPLC (column: Phenomenex Luna C18 80*40 mm*3 μm; mobile phase: [water (FA)-MeCN]; B %: 1%-40%, 8 min) to give the title compound as a white solid (35 mg, 33% yield, 98% purity). ¹H NMR (400 MHz, MeOD) δ=7.99 (s, 1H), 7.40 (d, J=1.6 Hz, 1H), 7.34 (dd, J=1.9, 8.4 Hz, 1H), 7.15 (d, J=8.4 Hz, 1H), 4.82 (br d, J=2.0 Hz, 1H), 4.57 (br d, J=13.2 Hz, 1H), 4.08-3.97 (m, 2H), 3.43 (s, 3H), 3.24-3.11 (m, 1H), 2.86 (t, J=12.4 Hz, 1H), 2.71 (t, J=12.8 Hz, 1H), 2.17-1.98 (m, 1H), 1.89-1.83 (m, 2H), 1.28 (s, 6H), 1.04 (d, J=6.8 Hz, 3H). LCMS: [M+H]⁺=510.3.

### Example 10: Degradation Activity in SU-DHL-4 Cells

### HiBiT Protocol

DC₅₀ (concentration to reach 50% degradation) values were determined from a cellular degradation assay (HiBiT, Promega™) in Su-DHL-4 cells (Table 1). Endogenous B cell lymphoma 6 (BCL6) was tagged with the 11-amino acid Small BiT (SmBiT) through clustered regularly interspaced short palindromic repeats (CRISPR)/CRISPR-associated protein 9 (Cas9) gene editing and single cell clone selection. After 24 hours of compound treatment, cells were lysed and incubated with Large BiT (LgBiT) protein to reconstitute intact nanoluciferase. Substrate was then added and relative luciferase units were measured. Degradation levels for each treatment were taken as a percentage compared to the control, 100% DMSO (Prism).

### Example 11: Kinetic Solubility

The preparation of 50 mM phosphate buffer (PB) with the pH at pH 7.4:
a) The preparation of 50 mM NaH₂PO₄: Dissolved 3.00 g of NaH₂PO₄ with 500 mL of water, and the pH measured was about 4.5;
b) The preparation of 50 mM Na₂HPO₄: Dissolved 3.55 g of Na₂HPO₄ with 500 mL of water, and the pH measured was about 9.4;
c) The preparation of 50 mM PB (pH 7.4): 15 mL of 50 mM Na₂HPO₄ was added into a 50 mL tube, and then adjusted pH to 7.4 with 50 mM NaH₂PO₄.

### Protocol:

a) 10 μL of 10 mM in DMSO of test and control compounds was added into lower chambers of Whatman® Mini-UniPrep® vials, respectively;
b) 490 μL of 50 mM PB (pH 7.4) was added into lower chambers of the Whatman® Mini-UniPrep® vials, respectively;
c) The solubility samples were vortexed for at least 2 minutes;
d) The Mini-UniPrep® vials were shook on a Barnstead shaker for 24 hours at room temperature at 800 rpm, and then centrifuged for 20 minutes (e.g. 4000 rpm);
e) Mini-UniPrep® vials were compressed to prepare the filtrates for injection into UPLC system to calculate the concentration with standard curve.

### Example 12: Metabolic Stability in Human Liver Microsome

### Preparation of Solutions

### a) Phosphate Buffer

K₂HPO₄ was dissolved in water to a final concentration of 50 mM, and then adjusted pH to 7.4 with HCl and filtered through a 0.22 μm filter.

### b) Quenching Solution

1 mg/mL stock of terfenadine/tolbutamide with DMSO was prepared, and then made into quenching solution (5/10 ng/mL terfenadine/tolbutamide) with can.

### c) Test Compound Working Solution

10 mM test compound stock with DMSO was prepared, and then made into 200 μM working solution with DMSO.

### d) Control Compound Working Solution

10 mM control compound (dextromethorphan) stock with DMSO was prepared, and then made into 200 μM working solution with DMSO.

### e) Liver Microsomes Working Solution

Liver microsomes were thawed in 37° C. water bath, and then made into 0.63 mg/mL liver microsomes working solution with phosphate buffer.

### f) NADPH Working Solution

5 mM NADPH working solution was prepared with phosphate buffer.

### Sample Preparation

a) 1.5 μL test compound/control compound working solution was added to 238.5 μL liver microsome (n=1).
b) The solution was pre-incubated at 37° C. for 5 min.
c) The reaction was started by adding 60 μL NADPH working solution.
d) At each time point (0, 5, 15, 30 and 60 min), 30 μL reaction mixture was removed and added to 300 μL quenching solution.
e) All samples were centrifuged at 4,000 rpm at 4° C. for 15 min, then 100 μL of supernatant was transferred with 100 μL water for LC-MS/MS analysis.

TABLE 1
Potency and Solubility Data

| Compound | 1 | 2a | 97 | 50 | 51a | BI-3802 | CCT-369260 | Cmp A | Cmp B |
|---|---|---|---|---|---|---|---|---|---|
| HiBiT SU- | 0.020 | 0.105 | 0.012 | 0.039 | 0.002 | 0.005 | 0.015 | 0.005 | 0.048 |
| DHL-4 DC₅₀ | | | | | | | | | |
| (μM) | | | | | | | | | |
| Kinetic | 108 | 265.5 | 403.5 | <1.56 | <1.56 | <1.56 | <1.56 | <1.56 | <1.56 |
| solubility | | | | | | | | | |
| @pH 7.4 | | | | | | | | | |
| (μM) | | | | | | | | | |
| Kinetic | 208 | NT | NT | 50.8 | 6.35 | 2.97 | 2.92 | <1.56 | <1.56 |
| solubility | | | | | | | | | |
| (FaSSIF, μM) | | | | | | | | | |
| Kinetic | 232 | NT | NT | 244 | 113 | 11.3 | 216 | 1.82 | 213 |
| solubility | | | | | | | | | |
| (FeSSIF, μM) | | | | | | | | | |
| Human Liver | <6.1 | <6.1 | 4.3 | 6.9 | <6.1 | 23.8 | 12.3 | <3.7 | 30.0 |
| Microsome | | | | | | | | | |
| Stability Clint | | | | | | | | | |
| (μL/min/mg) | | | | | | | | | |

*NT = not tested

Structures of known compounds:

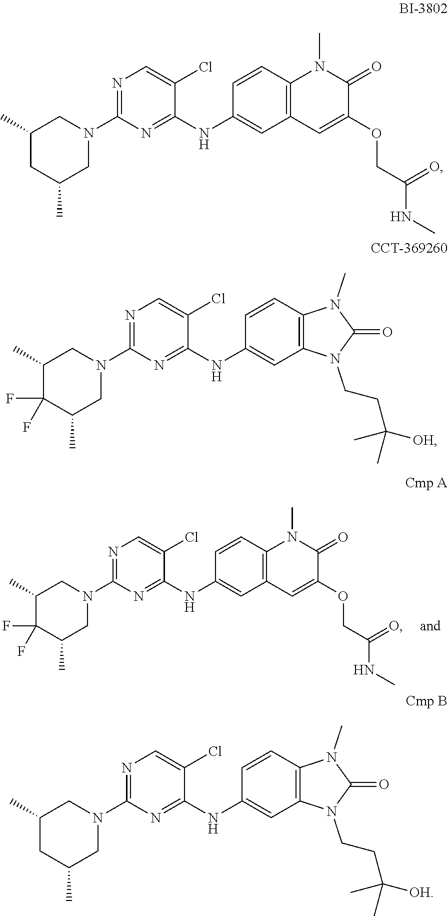

The results shown in Table 1 demonstrate that polar or charged moieties on the piperidine ring (compounds 1, 50, 51, and 97) resulted in degraders of BCL6. These results were surprising and unexpected in view of the report that polar or charged moieties generally result in non-degraders (Kerres, et al., Cell Rep. 20(12):2860-2875 (2017)). The introduction of the basic center resulted in maintaining/improving potency and/or improvement of kinetic solubility of the compounds.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I: 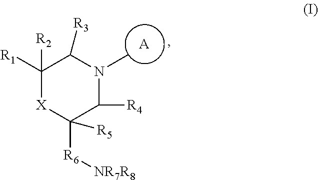
or a pharmaceutically acceptable salt or stereoisomer thereof,
wherein:
X is CH₂, S, CHF, CHCl, CHOH, or CF₂;
R₁ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
each R₉ is independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups;
each R₁₀ is independently alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups, or
R₁ and R₂ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₁₀ groups;
R₃ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₄ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₅ is hydrogen, cyano, halo, —OR₉, —N(R₉)₂, —C(O)N(R₉)₂, —SO₂N(R₉)₂, —N(R₉)C(O)R₉, —N(R₉)SO₂R₉, —N(R₉)C(O)N(R₉)₂, —P(O)(R₉)₂, (C₁-C₆)alkyl, (C₁-C₆)haloalkyl, (C₁-C₆)hydroxyalkyl, (C₃-C₇)carbocyclyl, 4- to 7-membered heterocyclyl, (C₆-C₁₀)aryl, monocyclic or bicyclic 5- to 10-membered heteroaryl, (C₂-C₆)alkenyl, or (C₂-C₆)alkynyl; wherein said alkyl, alkynyl, carbocyclyl, heterocyclyl, aryl, or heteroaryl is further optionally substituted by one or more, identical or different R₁₀ groups;
R₆ is absent, (C₁-C₆)alkylene, or (C₃-C₇)carbocyclyl; wherein said alkylene or carbocyclyl, is further optionally substituted by one or more, identical or different R₁₀ groups, or
R₅ and R₆ together with the same carbon atom to which they are attached form a spiro (C₃-C₇)carbocyclyl group or a 4- to 7-membered heterocyclyl group; wherein said carbocyclyl or heterocyclyl, is further optionally substituted by one or more, identical or different R₁₀ groups, or
R₆ is (C₂-C₄)alkylene which is bound to R₇ to form a 4- to 6-membered heterocyclyl group;
R₇ and R₈ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₁₀ groups, or
R₇ and R₈ together with the nitrogen atom to which they are attached form a 3- to 7-membered heterocyclyl, wherein said heterocyclyl is further optionally substituted by one or more, identical or different R₁₀ groups; 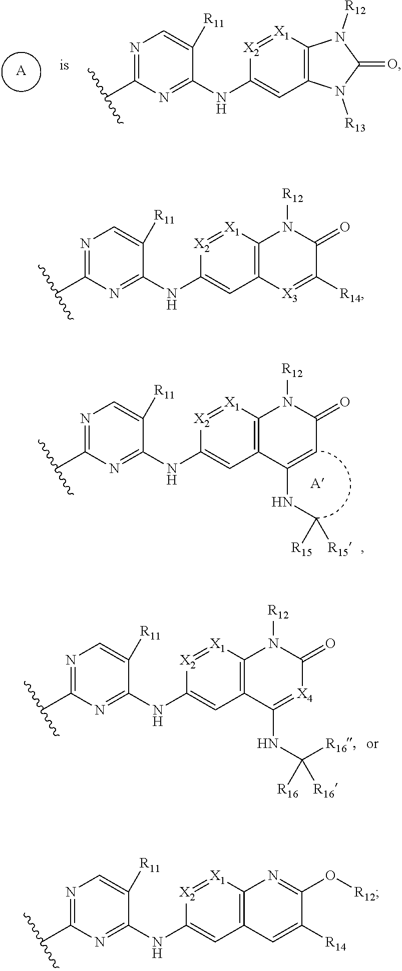
X₁ and X₂ are independently CR₁₇, or N;
R₁₇ is hydrogen, (C₁-C₄)alkyl, halo, hydroxy, amino, (C₁-C₄)alkoxy, (C₁-C₄)haloalkyl, (C₁-C₄)haloalkoxy, (C₂-C₄)alkenyl, (C₂-C₄)alkynyl, nitro, cyano, NH(C₁-C₄)alkyl, or N(C₁-C₄ alkyl)₂;
X₃ is CH or N;
X₄ is CR_{17′}, or N;
R_{17′} is hydrogen, fluor, chloro, or methyl;
R₁₁ is Cl or CN;
R₁₂ is hydrogen, (C₁-C₆)alkyl, (C₃-C₆)carbocyclyl, 4- to 7-membered heterocyclyl, (C₃-C₇)carbocyclyl(C₁-C₆)alkyl, or 4- to 7-membered heterocyclyl(C₁-C₆)alkyl; wherein said alkyl, carbocyclyl, or heterocyclyl is further optionally substituted by one or more, identical or different groups selected from (C₁-C₆)alkyl, (C₁-C₆)alkoxy, halo, amino, hydroxyl, haloalkyl, NH(C₁-C₆)alkyl, N((C₁-C₆)alkyl)₂, (C₃-C₆)carbocyclyl, and 4- to 7-membered heterocyclyl, or R₁₂ is -L-Y—Z or -L-Y—Z;
L is absent or (C₁-C₅)alkylene optionally substituted by one or more substituents selected from (C₁-C₂)alkyl and oxo;
Y is absent, O, S, S(O), S(O)₂, NR′, C(O), C(O)O, OC(O), C(O)N(R′), N(R′)C(O), N(R′)C(O)N(R′), N(R′)C(O)O, OC(O)N(R′), S(O)₂N(R′), or N(R′)S(O)₂;
each R′ is independently hydrogen or (C₁-C₄)alkyl;
Z is hydrogen, (C₁-C₆)alkyl, (C₂-C₆)alkenyl, (C₂-C₆)alkynyl, (C₃-C₁₀)carbocyclyl, or 3- to 10-membered heterocyclyl; wherein Z is optionally substituted by one or more substituents independently selected from (C₁-C₄)alkyl, halo, (C₁-C₄)haloalkyl, (C₁-C₄)haloalkoxy, amino, (C₁-C₄)aminoalkyl, cyano, hydroxy, carboxy, carbamoyl, sulphamoyl, mercapto, ureido, NR^{r}R^{s}, OR^{r}, C(O)R^{r}, C(O)ORᵣ, OC(O)R^{r}, C(O)NR^{r}R^{s}, N(R^{r})C(O)R^{r}, S(O)₀₋₂R^{r}, S(O)₂NR^{r}R^{s}, N(R^{r})SO₂R^{r}, Si(R^{r})(R^{s})R^{t} and (CH₂)₁₋₃NR^{r}R^{s}; wherein R^{r}, R^{s}, and R^{t} are each independently hydrogen, (C₁-C₆)alkyl, or (C₃-C₆)cycloalkyl; or R^{r} and R^{s} together with the nitrogen atom to which they are attached form a 4- to 9-membered heterocyclyl which is optionally substituted by one or more substituents selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, and hydroxy;
R₁₃ is hydrogen, methyl, —(CH₂)₁₋₃W₁W₂, or 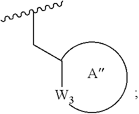
W₁ is CR₁₈R_{18′} or C(O);
R₁₈ and R_{18′} are independently hydrogen, (C₁-C₂)alkyl, fluoro, hydroxy, cyano, nitro, (C₁-C₂)alkoxy, (C₁-C₂)haloalkyl, (C₁-C₂)haloalkoxy, amino, NH(C₁-C₂)alkyl, or N(C₁-C₂ alkyl)₂, or
R₁₈ and R_{18′} together with the carbon atom to which they are attached form C(O), (C₃-C₆)carbocyclyl or 3- to 6-membered heterocyclyl, which is optionally substituted by one or more substituents independently selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
W₂ is cyano, hydroxy, 5- or 6-membered heteroaryl, phenyl, C(O)—(C₁-C₂)alkyl, S(O)₂—(C₁-C₂)alkyl, C(O)OCH₃, C(O)NHCH₃, CR₁₉R₂₀R₂₁, amino, NH(C₁-C₂)alkyl, or N(C₁-C₂ alkyl)₂:
R₁₉ is hydrogen, (C₁-C₂)alkyl, fluoro, chloro, bromo, hydroxy, amino, cyano, nitro, (C₁-C₂)alkoxy, (C₁-C₂)haloalkyl, or (C₁-C₂)haloalkoxy;
R₂₀ is hydrogen, (C₁-C₂)alkyl, fluoro, chloro, bromo, hydroxy, cyano, nitro, (C₁-C₂)alkoxy, (C₁-C₂)haloalkyl, (C₁-C₂)haloalkoxy, or —Y₂-L₂-Z₂;
Y₂ is absent, O, S, S(O), S(O)₂, NR′, C(O), C(O)O, OC(O), C(O)N(R′), N(R′)C(O), S(O)₂N(R′), or N(R′)SO₂;
L₂ is absent or (C₁-C₂)alkylene;
Z₂ is hydrogen, (C₁-C₆)alkyl, (C₂-C₄)alkenyl, (C₂-C₄)alkynyl, phenyl, (C₃-C₆)carbocyclyl, or 4- to 6-membered heterocyclyl, wherein Z₂ is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, cyano, hydroxy, C(O)R′, C(O)OR′, OC(O)R′, C(O)NR′R′, and N(R′)C(O)R′, wherein each R′ is independently hydrogen or (C₁-C₄)alkyl;
or R₁₉ and R₂₀ together with the carbon atom to which they are attached form (C₃-C₆)carbocyclyl or 3- to 6-membered heterocyclyl, optionally substituted by one or more substituents selected from (C₁-C₂)alkyl, halo, (C₁-C₂)haloalkyl, (C₁-C₂)haloalkoxy, (C₁-C₂)alkoxy, (C₁-C₂)alkylamino, amino, cyano, and hydroxy;
R₂₁ is (C₁-C₂)alkyl, —C(O)OR″, OR″, —C(O)NR″, NR″R″, phenyl, or 5-membered heteroaryl, wherein each R″ is independently hydrogen or (C₁-C₂)alkyl;
A″ is (C₄-C₆)carbocyclyl or 4- to 6-membered heterocyclyl, optionally substituted with one or more substituents independently selected from (C₁-C₂)alkyl, halo, hydroxy, cyano, and (C₁-C₂)alkoxy;
W₃ is NR₂₂ or CR₂₃R_{23′};
R₂₂ is hydrogen, (C₁-C₂)alkyl, (C₁-C₄)haloalkyl, (C₁-C₄)hydroxyalkyl, —C(O)CH₃, or —C(O)O—(C₁-C₄)alkyl;
R₂₃ and R_{23′} are independently hydrogen, (C₁-C₂)alkyl, cyclopropyl, fluoro, chloro, bromo, hydroxy, amino, cyano, nitro, (C₁-C₂)haloalkyl, (C₁-C₂)haloalkoxy, (C₁-C₂)alkoxy, —C(O)OR″, NR″R″, phenyl, or 5-membered heteroaryl;
R₁₄ is -L₃CR₂₄R₂₅R₂₆, or —CH═CH—R₂₆;
L₃ is absent, O, S, (C₁-C₄)alkylene, —O—(C₁-C₄)alkylene, or —S—(C₁-C₄)alkylene;
R₂₄ is hydrogen or (C₁-C₄)alkyl;
R₂₅ is hydrogen or (C₁-C₄)alkyl, or
R₂₄ and R₂₅ together with the carbon atom to which they are attached form a (C₃-C₅)carbocyclyl, 4- to 7-membered heterocyclyl, or C═O;
R₂₆ is (C₁-C₆)alkyl, —NR₂₇R₂₈, —OR₂₇, —C(O)R₂₇, —C(O)OR₂₇, —N(R₂₈)C(O)R₂₇, —C(O)NR₂₇R₂₈, —S(O)—(C₁-C₆)alkyl, —S(O)₂—(C₁-C₆)alkyl, —P(O)—(C₁-C₆ alkyl)₂, —C(NH)NH₂, or —(C₁-C₄)alkyl-NR₂₈C(O)R₂₇;
R₂₇ is hydrogen, 3- to 6-membered heterocyclyl, or (C₁-C₄)alkyl optionally substituted by one or more, identical or different groups selected from OH, Cl, F, CF₃, N(C₁-C₄ alkyl)₂, (C₃-C₆)carbocyclyl, 3- to 6-membered heterocyclyl, (C₂-C₄)alkenyl, and (C₂-C₄)alkynyl;
R₂₈ is hydrogen or (C₁-C₄)alkyl;
R₁₅ is hydrogen, (C₁-C₄)alkyl, (C₃-C₆)cycloalkyl, (C₁-C₄)haloalkyl, or cyano, wherein said alkyl or cycloalkyl is optionally substituted by one or more substituents selected from (C₁-C₄)alkyl, (C₃-C₆)cycloalkyl, hydroxy, (C₁-C₂)alkoxy, amino, NH(C₁-C₂)alkyl, N((C₁-C₂)alkyl)₂, (C₁-C₂)aminoalkyl, and halo;
R₁₅′ is hydrogen, (C₁-C₄)alkyl, cyano, (C₁-C₄)haloalkyl, or —Y₃-L₄-Z₃;
Y₃ is absent, C(O)O, or C(O)N(R′);
L₄ is absent or (C₁-C₂)alkylene;
Z₃ is hydrogen, (C₁-C₆)alkyl, phenyl, (C₃-C₆)cycloalkyl, or 4- to 6-membered heterocyclyl, wherein Z₃ is optionally substituted by one or more substituents independently selected from (C₁-C₂)alkyl, halo, (C₁-C₂)haloalkyl, (C₁-C₂)haloalkoxy, (C₁-C₂)alkoxy, amino, nitro, cyano, and hydroxy, or
R₁₅ and R₁₅′, together with the carbon atom to which they are attached, form a (C₄-C₆)carbocyclyl, or 4- to 6-membered heterocyclyl;
A′ is a 6- or 7-membered heterocyclyl, which in addition to R₁₅ and R₁₅′, is optionally further substituted by one more substituents independently selected from oxo, (C₁-C₂)alkyl, cyclopropyl, spiro-cyclopropyl, halo, (C₁-C₂)haloalkyl, (C₁-C₂)haloalkoxy, (C₁-C₂)alkoxy, amino, cyano, and hydroxy;
R₁₆ is hydrogen, (C₁-C₂)alkyl, (C₃-C₄)cycloalkyl, (C₁-C₂)haloalkyl, cyano, (C₂-C₄)alkenyl, or (C₂-C₄)alkynyl;
R₁₆′ is (C₁-C₄)alkyl, cyano, (C₁-C₄)haloalkyl, or —Y₄-L₅-Z₄;
Y₄ is absent, C(O), C(O)O, OC(O), C(O)N(R′), or S(O)₂N(R′);
L₅ is absent or (C₁-C₂)alkylene optionally substituted by one or more substituents selected from (C₁-C₂)alkyl and oxo;
Z₄ is hydrogen, (C₁-C₆)alkyl, (C₂-C₄)alkenyl, (C₂-C₄)alkynyl, phenyl, (C₃-C₆)carbocyclyl, (C₃-C₆)cycloalkenyl, or 4- to 6-membered heterocyclyl, wherein Z₄ is optionally substituted by one or more substituents independently selected from oxo, (C₁-C₄)alkyl, (C₃-C₆)cycloalkyl, halo, (C₁-C₄)haloalkyl, (C₁-C₄)haloalkoxy, (C₁-C₄)alkoxy, (C₁-C₄)alkylamino, amino, nitro, cyano, hydroxy, C(O)Rᵤ, C(O)OR^{u}, OC(O)R^{u}, C(O)NR^{u}R^{u}, and N(R^{u})C(O)R^{u}, wherein each R^{u} is independently hydrogen, (C₁-C₄)alkyl, or (C₃-C₆)cycloalkyl, or
Z₄ is -Q-L₆-W, wherein
Q is absent, O, NH, or N(C₁-C₂)alkyl;
L₆ is absent or (C₁-C₂)alkylene optionally substituted by one or more substituents selected from oxo and (C₁-C₂)alkyl;
W is (C₁-C₄)alkyl, phenyl, (C₃-C₆)cycloalkyl, (C₃-C₆)cycloalkenyl, or 5- or 6-membered heterocyclyl, wherein W is optionally substituted by one or more substituents independently selected from (C₁-C₄)alkyl, halo, (C₁-C₄)haloalkyl, (C₁-C₄)haloalkoxy, (C₁-C₄)alkoxy, (C₁-C₄)alkylamino, amino, nitro, cyano, or hydroxy, or
R₁₆ and R₁₆′, together with the carbon atom to which they are attached, for a (C₃-C₁₀)carbocyclyl or a 4- to 10-membered heterocyclyl, which is optionally substituted by one or more substituents independently selected from oxo, (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; or the (C₃-C₁₀)carbocyclyl or 4- to 10-membered heterocyclyl is optionally fused to a 5- or 6-membered heteroaryl or phenyl ring, and the 5- or 6-membered heteroaryl or phenyl ring is optionally substituted by (C₁-C₂)alkyl, halo, (C₁-C₂)haloalkyl, (C₁-C₂)haloalkoxy, (C₁-C₂)alkoxy, (C₁-C₂)alkylamino, amino, nitro, cyano, or hydroxy; and
R₁₆″ is hydrogen, (C₁-C₄)alkyl, (C₁-C₂)haloalkyl, (C₁-C₂)alkoxy, (C₁-C₂)haloalkoxy, cyano, nitro, acetylenyl, phenyl, or 5- or 6-membered heteroaryl, wherein said alkyl, phenyl, or heteroaryl is optionally substituted by one or more substituents independently selected from halo, hydroxy, and amino;
provided that when R₇ is (C₁-C₃)alkyl and R₈ is (C₁-C₃)alkyl, R₆ is not (C₁-C₆)alkylene, and provided that when 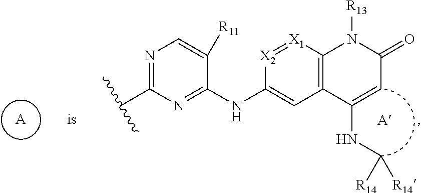
R₁₁ is Cl, X is CH₂ or CF₂, and R₆ is absent or optionally substituted (C₁-C₂)alkylene, R₇ and R₈ cannot be a) independently hydrogen or (C₁-C₂)alkyl optionally substituted with an oxo group, or b) R₇ and R₈ together with the nitrogen atom to which they are attached form a 4-membered heterocyclyl optionally substituted with a gem-difluoro group.

2. The compound of claim 1, wherein is 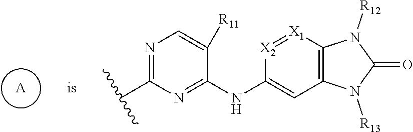
and the compound has the structure of formula I-1,

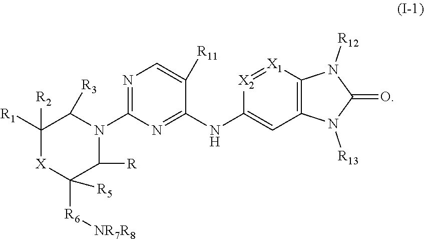

3. The compound of claim 1, wherein 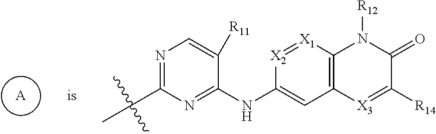
and the compound has the structure of formula I-2,

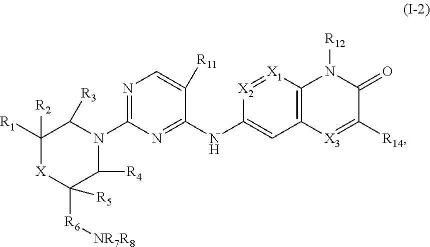

4. The compound of claim 1, wherein 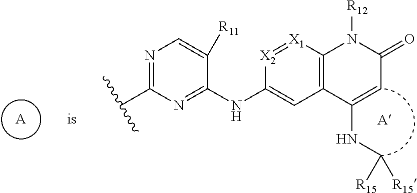
and the compound has the structure of formula I-3,

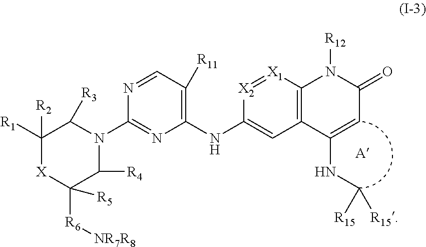

5. The compound of claim 1, wherein is 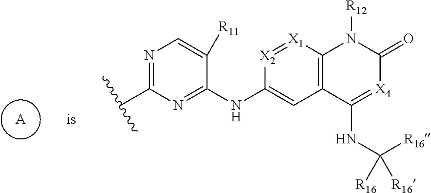
and the compound has the structure of formula I-4,

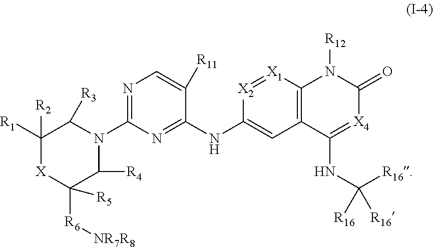

6. The compound of claim 1, wherein 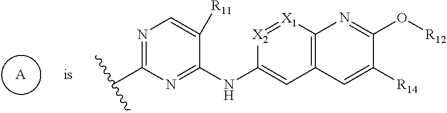
and the compound has the structure of formula I-5,

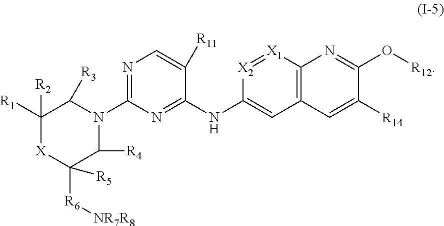

7. The compound of claim 1, wherein R₁₁ is Cl.

8. The compound of claim 1, wherein R₁₁ is CN.

9. The compound of claim 1, wherein R₁ is hydrogen, —OR₉, or (C₁-C₆)alkyl.

10. The compound of claim 9, wherein R₁ is hydrogen, methyl, or —OH.

11. The compound of claim 10, wherein R₁ is hydrogen.

12. The compound of claim 10, wherein Ri is methyl.

13. The compound of claim 1, wherein R₂ is hydrogen, —OR₉, or (C₁-C₆)alkyl.

14. The compound of claim 13, wherein R₂ is hydrogen, methyl, or —OH.

15. The compound of claim 14, wherein R₂ is hydrogen.

16. The compound of claim 14, wherein R₂ is methyl.

17. The compound of claim 1, wherein R₁ and R₂ together with the same carbon atom to which they are attached form a spiro (C₃-C₇)carbocyclyl group.

18. The compound of claim 17, wherein R₁ and R₂ together with the same carbon atom to which they are attached form a spiro (C₃)carbocyclyl.

19. The compound of claim 1, wherein R₃ is hydrogen.

20. The compound of claim 1, wherein R₄ is hydrogen.

21. The compound of claim 1, wherein R₅ is hydrogen, —OR₉, or (C₁-C₆)alkyl.

22. The compound of claim 21, wherein R₅ is hydrogen, methyl, or —OH.

23. The compound of claim 22, wherein R₅ is hydrogen.

24. The compound of claim 22, wherein R₅ is methyl.

25. The compound of claim 1, wherein R₆ is optionally substituted (C₁-C₆)alkylene or optionally substituted (C₃-C₇)carbocyclyl.

26. The compound of claim 25, wherein R₆ is optionally substituted (C₁-C₂)alkylene or optionally substituted (C₃-C₄)carbocyclyl.

27. The compound of claim 26, wherein R₆ is Cl-alkylene.

28. The compound of claim 26, wherein R₆ is C₂-alkylene.

29. The compound of claim 26, wherein R₆ is Cl-alkylene substituted by one or more, identical or different R₁₀ groups.

30. The compound of claim 26, wherein R₆ is C₂-alkylene substituted by one or more, identical or different R₁₀ groups.

31. The compound of claim 29, wherein each R₁₀ is methyl, fluoro, or cyclopropyl.

32. The compound of claim 26, wherein R₆ is C₃-carbocyclyl.

33. The compound of claim 26, wherein R₆ is C₄-carbocyclyl.

34. The compound of claim 1, wherein R₅ and R₆ together with the same carbon atom to which they are attached form a spiro (C₃-C₇)carbocyclyl group.

35. The compound of claim 34, wherein R₅ and R₆ together with the same carbon atom to which they are attached form a spiro (C₃)carbocyclyl.

36. The compound of claim 1, wherein R₆ is (C₂-C₄)alkylene which is bound to R₇ to form a 4- to 6-membered heterocyclyl group.

37. The compound of claim 36, wherein R₆ is (C₂-C₃)alkylene which is bound to R₇ to form a 4-membered heterocyclyl group.

38. The compound of claim 1, wherein R₇ is hydrogen or (C₁-C₆)alkyl.

39. The compound of claim 38, wherein R₇ is hydrogen or methyl.

40. The compound of claim 1, wherein R₅ is hydrogen or (C₁-C₆)alkyl.

41. The compound of claim 40, wherein R₅ is hydrogen or methyl.

42. The compound of claim 1, wherein R₇ and R₈ together with the nitrogen atom to which they are attached form a 3- to 7-membered heterocyclyl.

43. The compound of claim 42, wherein R₇ and R₈ together with the nitrogen atom to which they are attached form a 3-membered heterocyclyl.

44. The compound of claim 1, wherein X is CH₂.

45. The compound of claim 1, wherein X is CHI.

46. The compound of claim 1, wherein X is CF₂.

47. The compound of claim 1, which is: 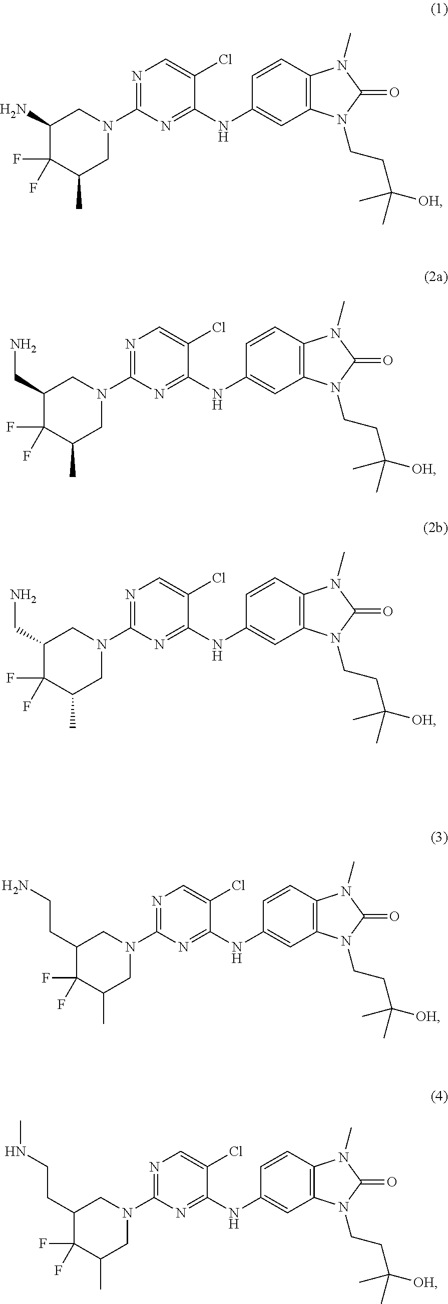 [SMILES: C[C@@H]1CN(c2ncc(Cl)c(Nc3ccc4c(c3)n(CCC(C)(C)O)c(=O)n4C)n2)C[C@H](N)C1(F)F ; C[C@@H]1CN(c2ncc(Cl)c(Nc3ccc4c(c3)n(CCC(C)(C)O)c(=O)n4C)n2)C[C@H](CN)C1(F)F ; C[C@H]1CN(c2ncc(Cl)c(Nc3ccc4c(c3)n(CCC(C)(C)O)c(=O)n4C)n2)C[C@@H](CN)C1(F)F ; CC1CN(c2ncc(Cl)c(Nc3ccc4c(c3)n(CCC(C)(C)O)c(=O)n4C)n2)CC(CCN)C1(F)F ; CNCCC1CN(c2ncc(Cl)c(Nc3ccc4c(c3)n(CCC(C)(C)O)c(=O)n4C)n2)CC(C)C1(F)F] 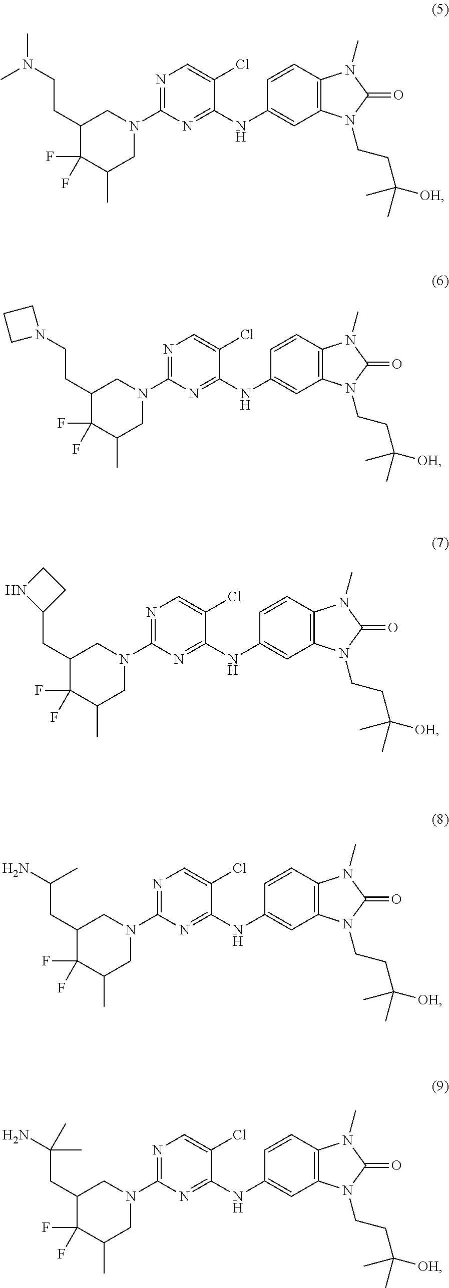 [SMILES: CC1CN(c2ncc(Cl)c(Nc3ccc4c(c3)n(CCC(C)(C)O)c(=O)n4C)n2)CC(CCN(C)C)C1(F)F ; CC1CN(c2ncc(Cl)c(Nc3ccc4c(c3)n(CCC(C)(C)O)c(=O)n4C)n2)CC(CCN2CCC2)C1(F)F ; CC1CN(c2ncc(Cl)c(Nc3ccc4c(c3)n(CCC(C)(C)O)c(=O)n4C)n2)CC(CC2CCN2)C1(F)F ; CC(N)CC1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(CC(C)(C)N)C1(F)F] 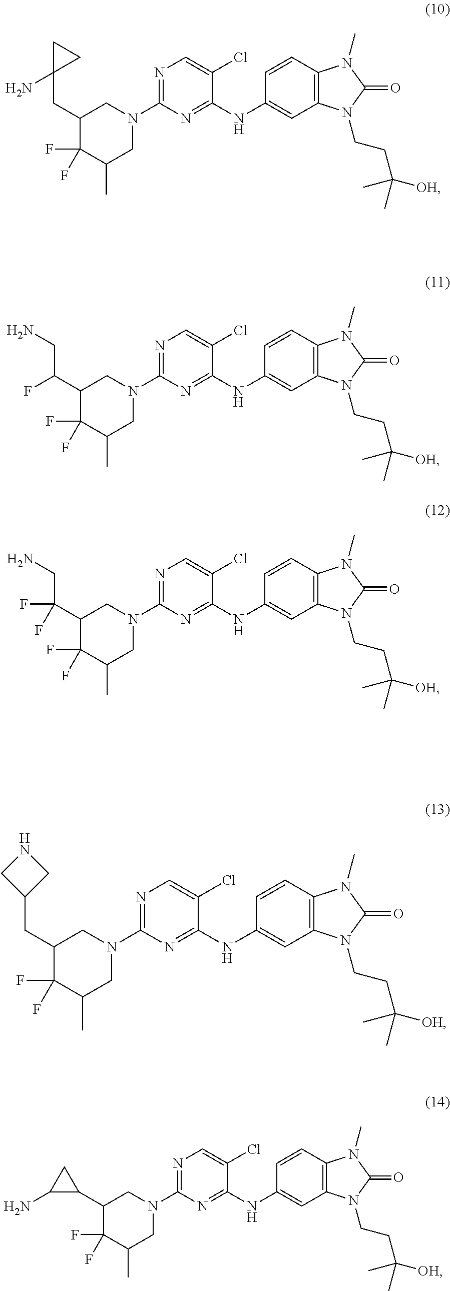 [SMILES: CC1CN(c2ncc(Cl)c(Nc3ccc4c(c3)n(CCC(C)(C)O)c(=O)n4C)n2)CC(CC2(N)CC2)C1(F)F ; CC1CN(c2ncc(Cl)c(Nc3ccc4c(c3)n(CCC(C)(C)O)c(=O)n4C)n2)CC(C(F)CN)C1(F)F ; CC1CN(c2ncc(Cl)c(Nc3ccc4c(c3)n(CCC(C)(C)O)c(=O)n4C)n2)CC(C(F)(F)CN)C1(F)F ; CC1CN(c2ncc(Cl)c(Nc3ccc4c(c3)n(CCC(C)(C)O)c(=O)n4C)n2)CC(CC2CNC2)C1(F)F ; CC1CN(c2ncc(Cl)c(Nc3ccc4c(c3)n(CCC(C)(C)O)c(=O)n4C)n2)CC(C2CC2N)C1(F)F] 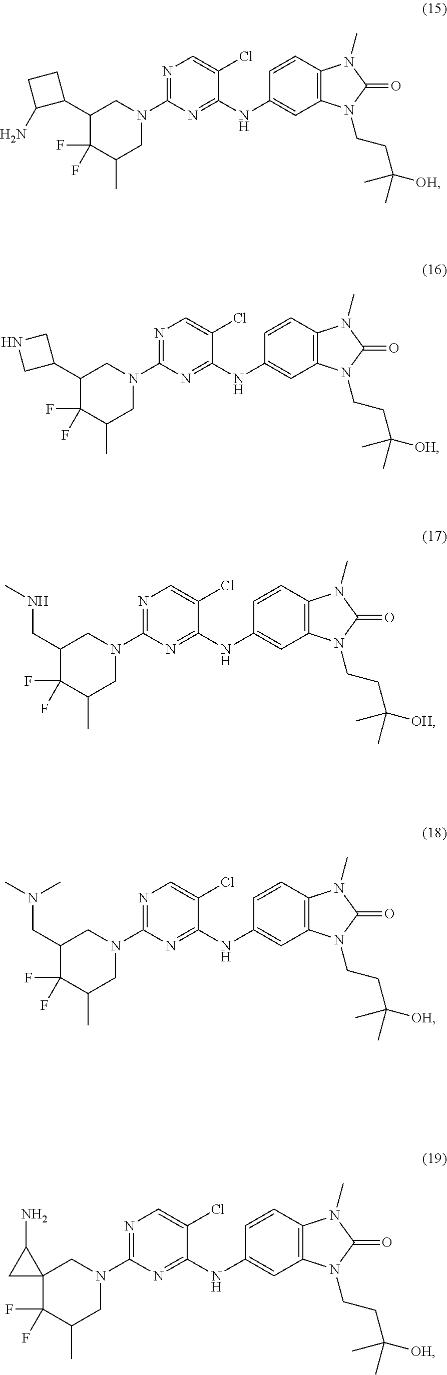 [SMILES: CC1CN(c2ncc(Cl)c(Nc3ccc4c(c3)n(CCC(C)(C)O)c(=O)n4C)n2)CC(C2CCC2N)C1(F)F ; CC1CN(c2ncc(Cl)c(Nc3ccc4c(c3)n(CCC(C)(C)O)c(=O)n4C)n2)CC(C2CNC2)C1(F)F ; CNCC1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(CN(C)C)C1(F)F ; CC1CN(c2ncc(Cl)c(Nc3ccc4c(c3)n(CCC(C)(C)O)c(=O)n4C)n2)CC2(CC2N)C1(F)F] 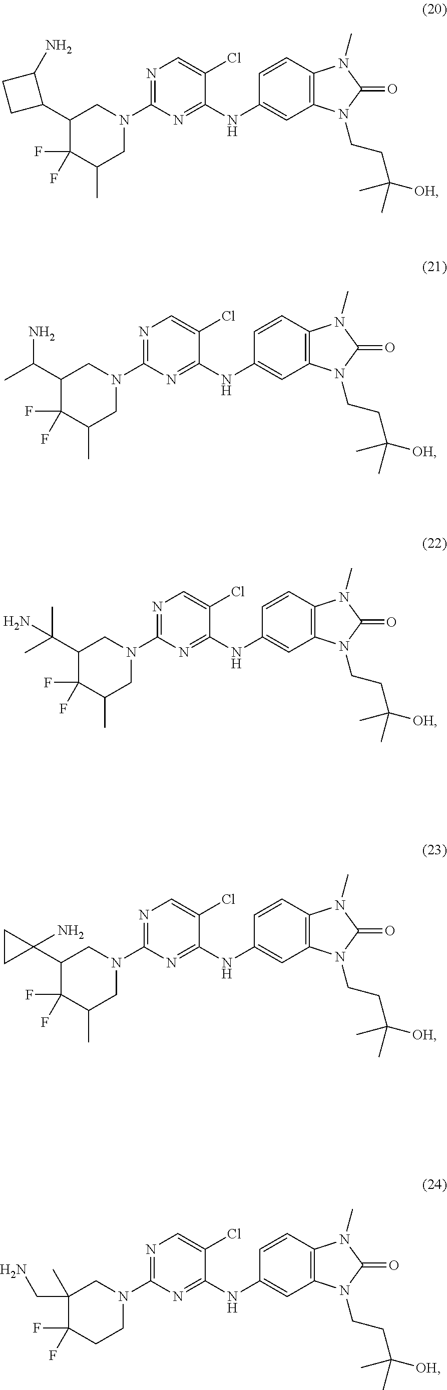 [SMILES: CC1CN(c2ncc(Cl)c(Nc3ccc4c(c3)n(CCC(C)(C)O)c(=O)n4C)n2)CC(C2CCC2N)C1(F)F ; CC(N)C1CN(c2ncc(Cl)c(Nc3ccc4c(c3)n(CCC(C)(C)O)c(=O)n4C)n2)CC(C)C1(F)F ; CC1CN(c2ncc(Cl)c(Nc3ccc4c(c3)n(CCC(C)(C)O)c(=O)n4C)n2)CC(C(C)(C)N)C1(F)F ; CC1CN(c2ncc(Cl)c(Nc3ccc4c(c3)n(CCC(C)(C)O)c(=O)n4C)n2)CC(C2(N)CC2)C1(F)F ; Cn1c(=O)n(CCC(C)(C)O)c2cc(Nc3nc(N4CCC(F)(F)C(C)(CN)C4)ncc3Cl)ccc21] 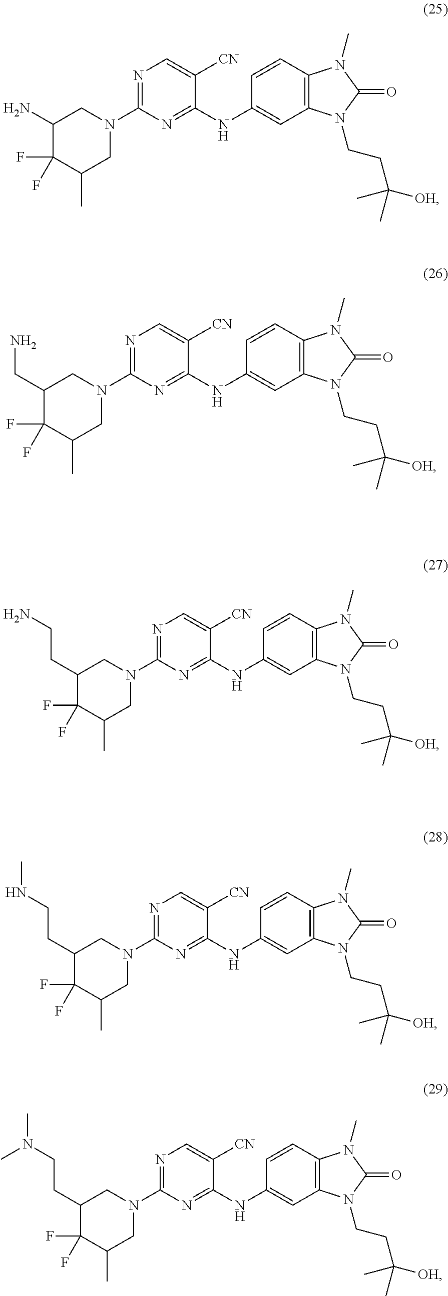 [SMILES: CC1CN(c2ncc(C#N)c(Nc3ccc4c(c3)n(CCC(C)(C)O)c(=O)n4C)n2)CC(N)C1(F)F ; CC1CN(c2ncc(C#N)c(Nc3ccc4c(c3)n(CCC(C)(C)O)c(=O)n4C)n2)CC(CN)C1(F)F ; CC1CN(c2ncc(C#N)c(Nc3ccc4c(c3)n(CCC(C)(C)O)c(=O)n4C)n2)CC(CCN)C1(F)F ; CNCCC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CCN(C)C)C1(F)F] 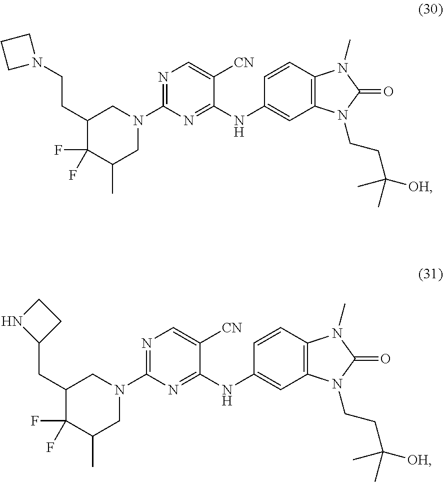 [SMILES: CC1CN(c2ncc(C#N)c(Nc3ccc4c(c3)n(CCC(C)(C)O)c(=O)n4C)n2)CC(CCN2CCC2)C1(F)F ; CC1CN(c2ncc(C#N)c(Nc3ccc4c(c3)n(CCC(C)(C)O)c(=O)n4C)n2)CC(CC2CCN2)C1(F)F] 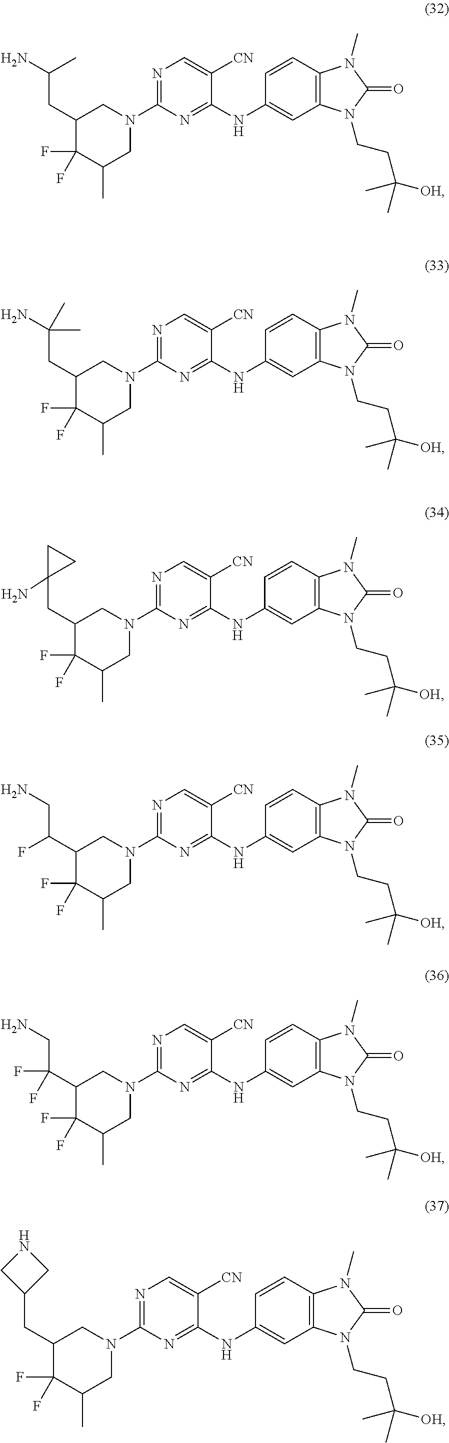 [SMILES: CC(N)CC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CC(C)(C)N)C1(F)F ; CC1CN(c2ncc(C#N)c(Nc3ccc4c(c3)n(CCC(C)(C)O)c(=O)n4C)n2)CC(CC2(N)CC2)C1(F)F ; CC1CN(c2ncc(C#N)c(Nc3ccc4c(c3)n(CCC(C)(C)O)c(=O)n4C)n2)CC(C(F)CN)C1(F)F ; CC1CN(c2ncc(C#N)c(Nc3ccc4c(c3)n(CCC(C)(C)O)c(=O)n4C)n2)CC(C(F)(F)CN)C1(F)F ; CC1CN(c2ncc(C#N)c(Nc3ccc4c(c3)n(CCC(C)(C)O)c(=O)n4C)n2)CC(CC2CNC2)C1(F)F] 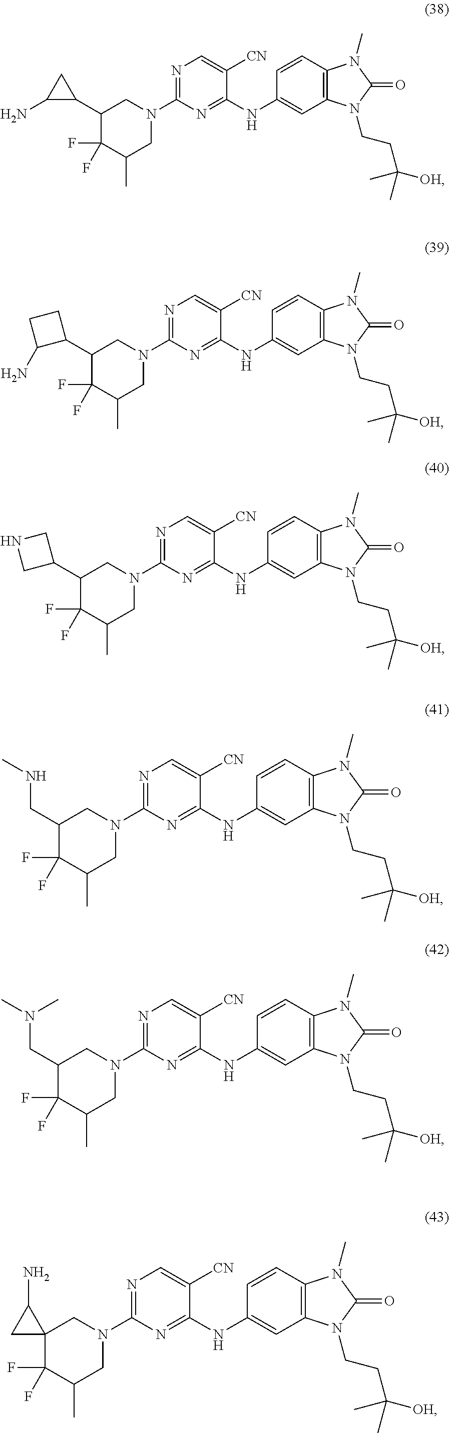 [SMILES: CC1CN(c2ncc(C#N)c(Nc3ccc4c(c3)n(CCC(C)(C)O)c(=O)n4C)n2)CC(C2CC2N)C1(F)F ; CC1CN(c2ncc(C#N)c(Nc3ccc4c(c3)n(CCC(C)(C)O)c(=O)n4C)n2)CC(C2CCC2N)C1(F)F ; CC1CN(c2ncc(C#N)c(Nc3ccc4c(c3)n(CCC(C)(C)O)c(=O)n4C)n2)CC(C2CNC2)C1(F)F ; CNCC1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(CN(C)C)C1(F)F ; CC1CN(c2ncc(C#N)c(Nc3ccc4c(c3)n(CCC(C)(C)O)c(=O)n4C)n2)CC2(CC2N)C1(F)F] 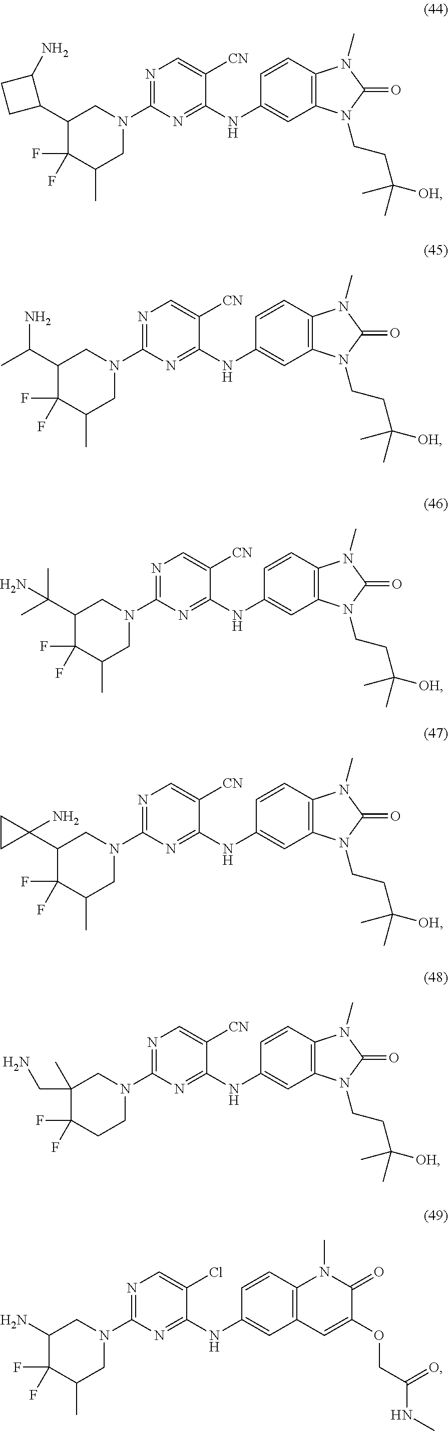 [SMILES: CC1CN(c2ncc(C#N)c(Nc3ccc4c(c3)n(CCC(C)(C)O)c(=O)n4C)n2)CC(C2CCC2N)C1(F)F ; CC(N)C1CN(c2ncc(C#N)c(Nc3ccc4c(c3)n(CCC(C)(C)O)c(=O)n4C)n2)CC(C)C1(F)F ; CC1CN(c2ncc(C#N)c(Nc3ccc4c(c3)n(CCC(C)(C)O)c(=O)n4C)n2)CC(C(C)(C)N)C1(F)F ; CC1CN(c2ncc(C#N)c(Nc3ccc4c(c3)n(CCC(C)(C)O)c(=O)n4C)n2)CC(C2(N)CC2)C1(F)F ; Cn1c(=O)n(CCC(C)(C)O)c2cc(Nc3nc(N4CCC(F)(F)C(C)(CN)C4)ncc3C#N)ccc21 ; CNC(=O)COc1cc2cc(Nc3nc(N4CC(C)C(F)(F)C(N)C4)ncc3Cl)ccc2n(C)c1=O] 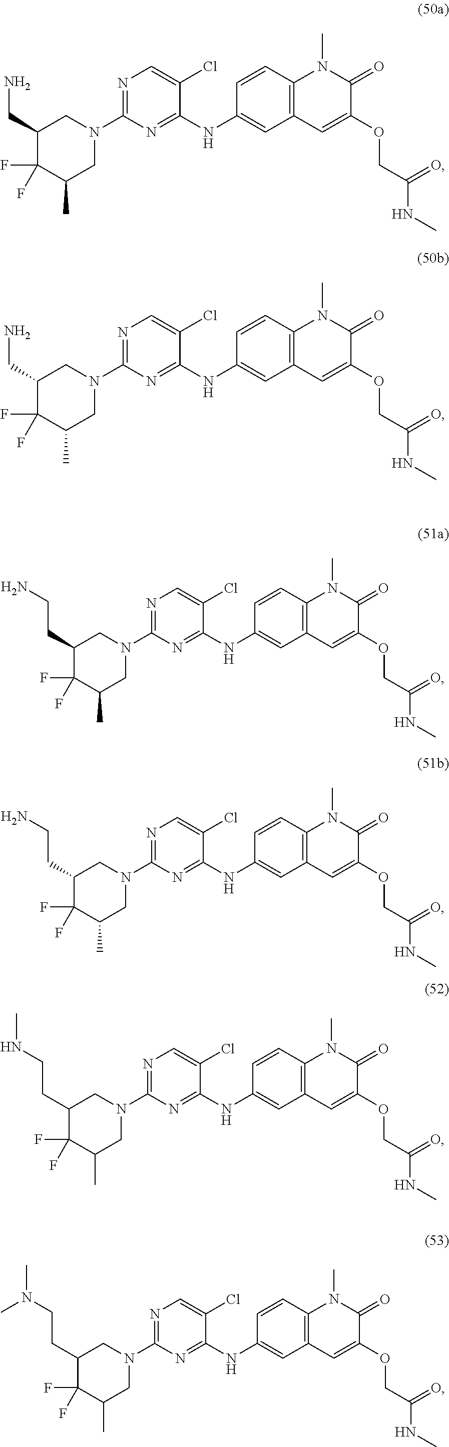 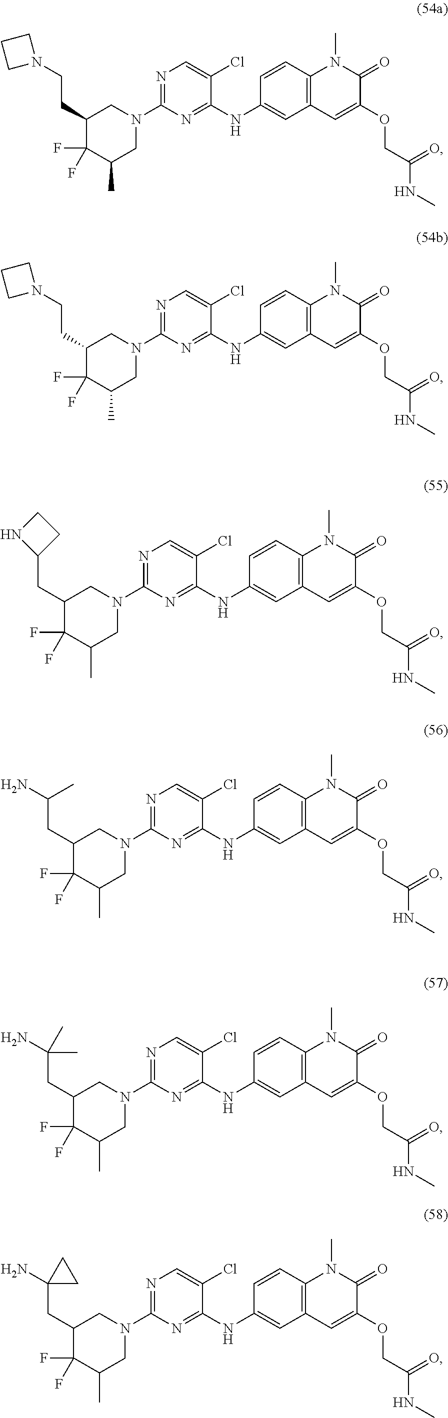 [SMILES: CNC(=O)COc1cc2cc(Nc3nc(N4C[C@H](CCN5CCC5)C(F)(F)[C@H](C)C4)ncc3Cl)ccc2n(C)c1=O ; CNC(=O)COc1cc2cc(Nc3nc(N4C[C@@H](CCN5CCC5)C(F)(F)[C@@H](C)C4)ncc3Cl)ccc2n(C)c1=O ; CNC(=O)COc1cc2cc(Nc3nc(N4CC(C)C(F)(F)C(CC5CCN5)C4)ncc3Cl)ccc2n(C)c1=O ; CNC(=O)COc1cc2cc(Nc3nc(N4CC(C)C(F)(F)C(CC(C)N)C4)ncc3Cl)ccc2n(C)c1=O ; CNC(=O)COc1cc2cc(Nc3nc(N4CC(C)C(F)(F)C(CC(C)(C)N)C4)ncc3Cl)ccc2n(C)c1=O ; CNC(=O)COc1cc2cc(Nc3nc(N4CC(C)C(F)(F)C(CC5(N)CC5)C4)ncc3Cl)ccc2n(C)c1=O] 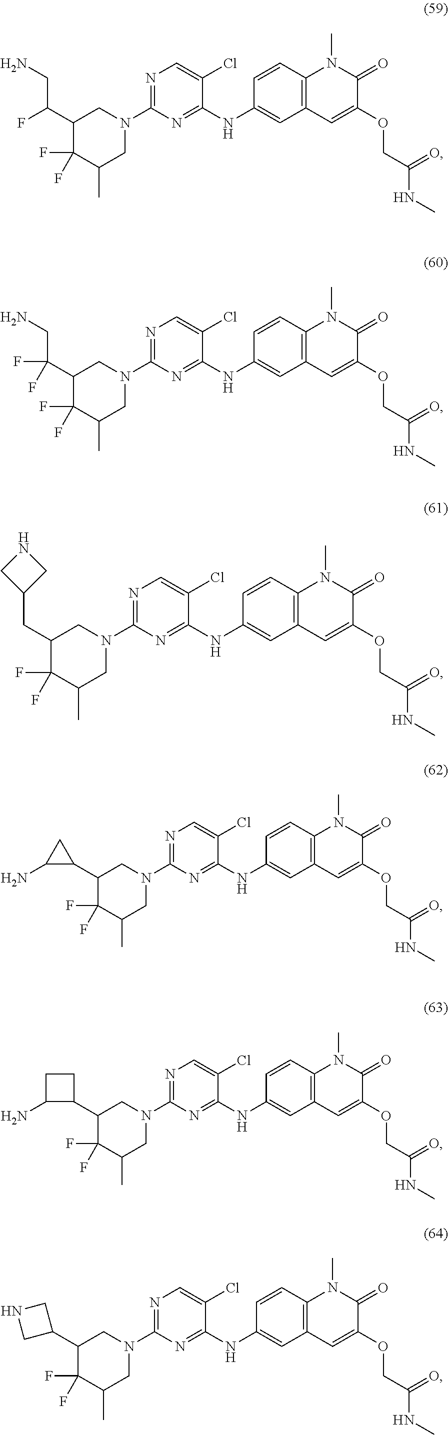 [SMILES: CNC(=O)COc1cc2cc(Nc3nc(N4CC(C)C(F)(F)C(C(F)CN)C4)ncc3Cl)ccc2n(C)c1=O ; CNC(=O)COc1cc2cc(Nc3nc(N4CC(C)C(F)(F)C(C(F)(F)CN)C4)ncc3Cl)ccc2n(C)c1=O ; CNC(=O)COc1cc2cc(Nc3nc(N4CC(C)C(F)(F)C(CC5CNC5)C4)ncc3Cl)ccc2n(C)c1=O ; CNC(=O)COc1cc2cc(Nc3nc(N4CC(C)C(F)(F)C(C5CC5N)C4)ncc3Cl)ccc2n(C)c1=O ; CNC(=O)COc1cc2cc(Nc3nc(N4CC(C)C(F)(F)C(C5CCC5N)C4)ncc3Cl)ccc2n(C)c1=O ; CNC(=O)COc1cc2cc(Nc3nc(N4CC(C)C(F)(F)C(C5CNC5)C4)ncc3Cl)ccc2n(C)c1=O] 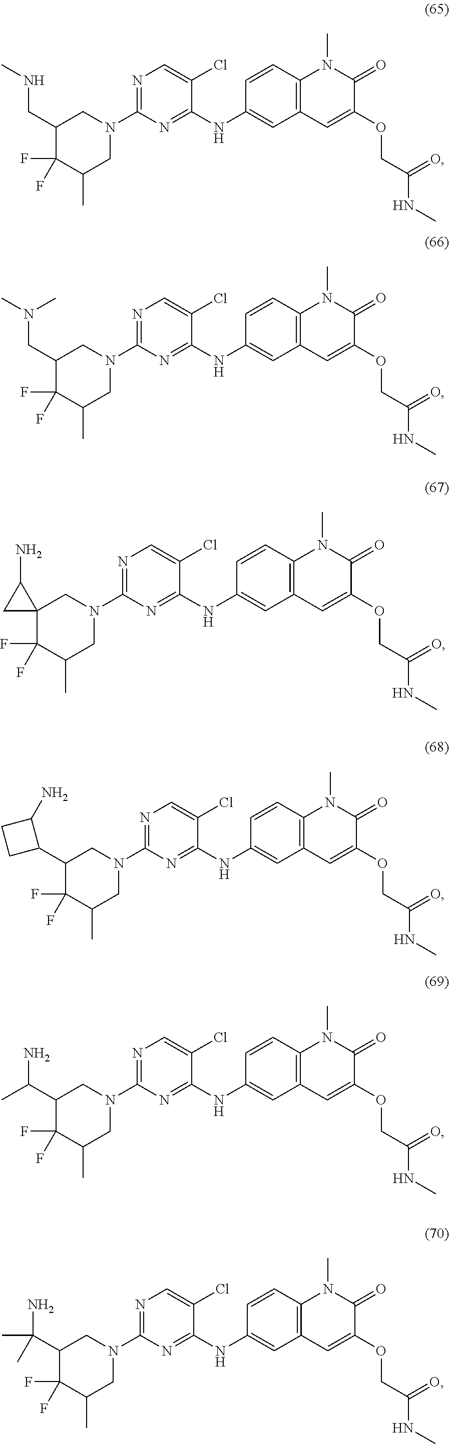 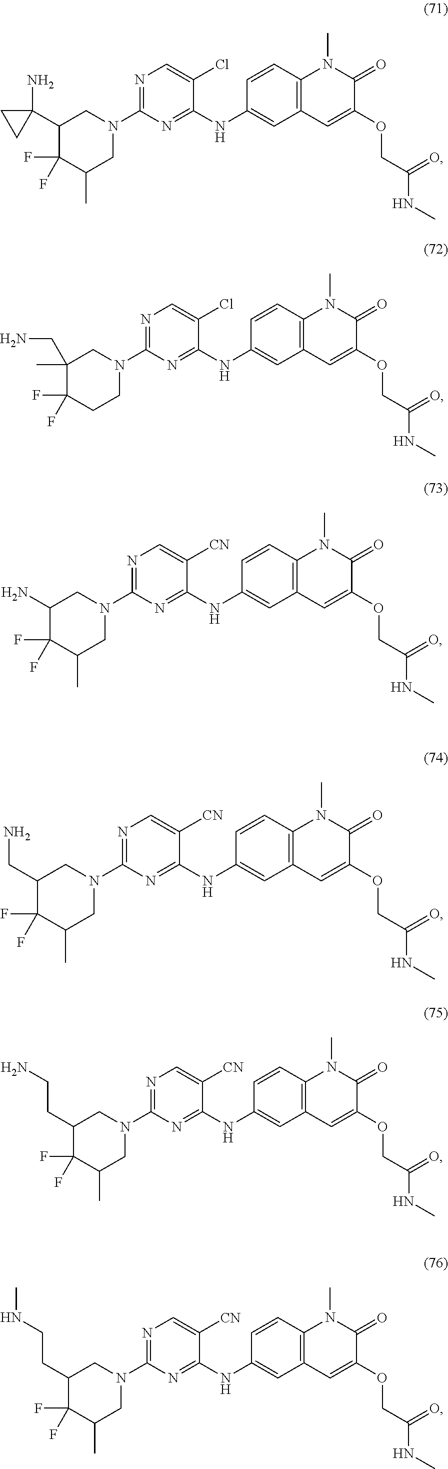 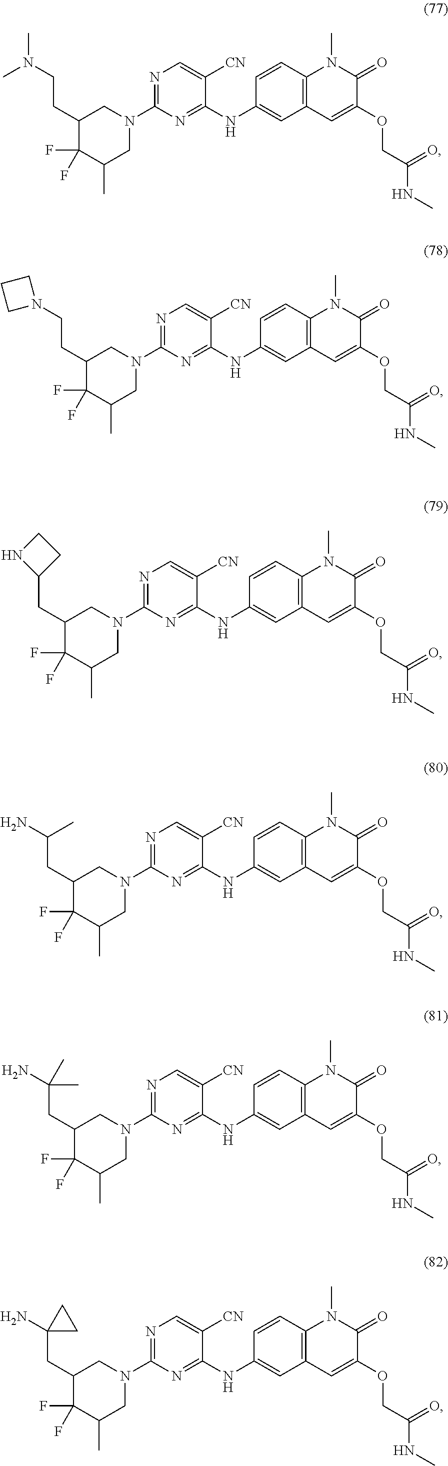 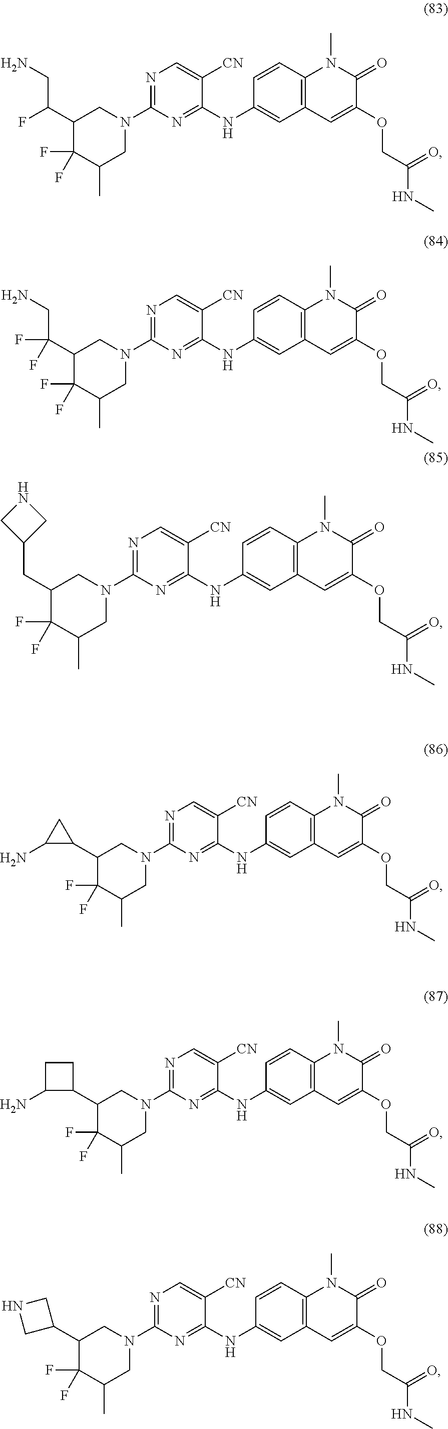 [SMILES: CNC(=O)COc1cc2cc(Nc3nc(N4CC(C)C(F)(F)C(C(F)CN)C4)ncc3C#N)ccc2n(C)c1=O ; CNC(=O)COc1cc2cc(Nc3nc(N4CC(C)C(F)(F)C(C(F)(F)CN)C4)ncc3C#N)ccc2n(C)c1=O ; CNC(=O)COc1cc2cc(Nc3nc(N4CC(C)C(F)(F)C(CC5CNC5)C4)ncc3C#N)ccc2n(C)c1=O ; CNC(=O)COc1cc2cc(Nc3nc(N4CC(C)C(F)(F)C(C5CC5N)C4)ncc3C#N)ccc2n(C)c1=O ; CNC(=O)COc1cc2cc(Nc3nc(N4CC(C)C(F)(F)C(C5CCC5N)C4)ncc3C#N)ccc2n(C)c1=O ; CNC(=O)COc1cc2cc(Nc3nc(N4CC(C)C(F)(F)C(C5CNC5)C4)ncc3C#N)ccc2n(C)c1=O] 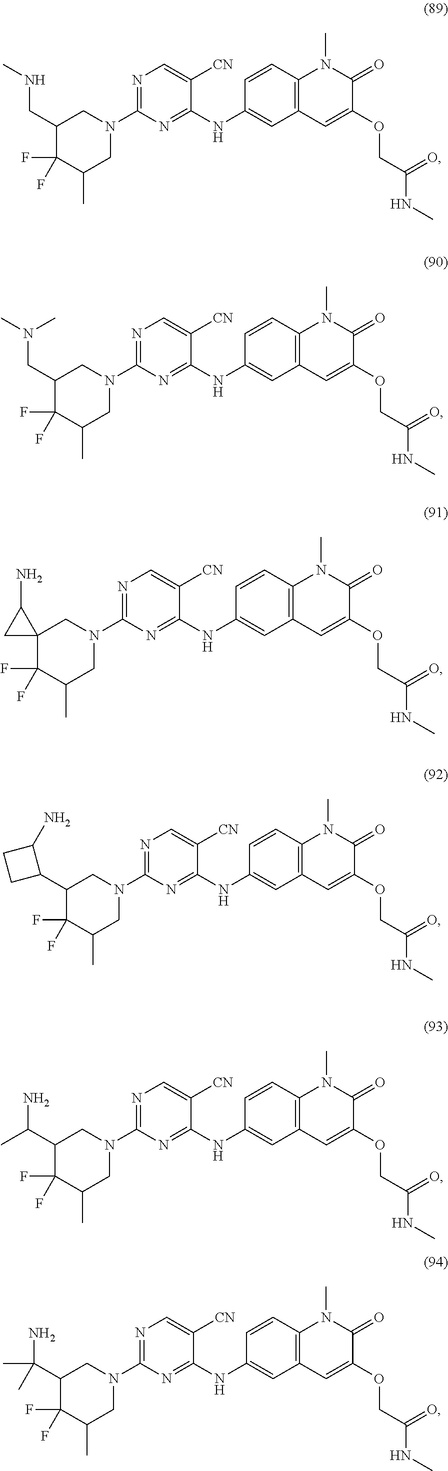 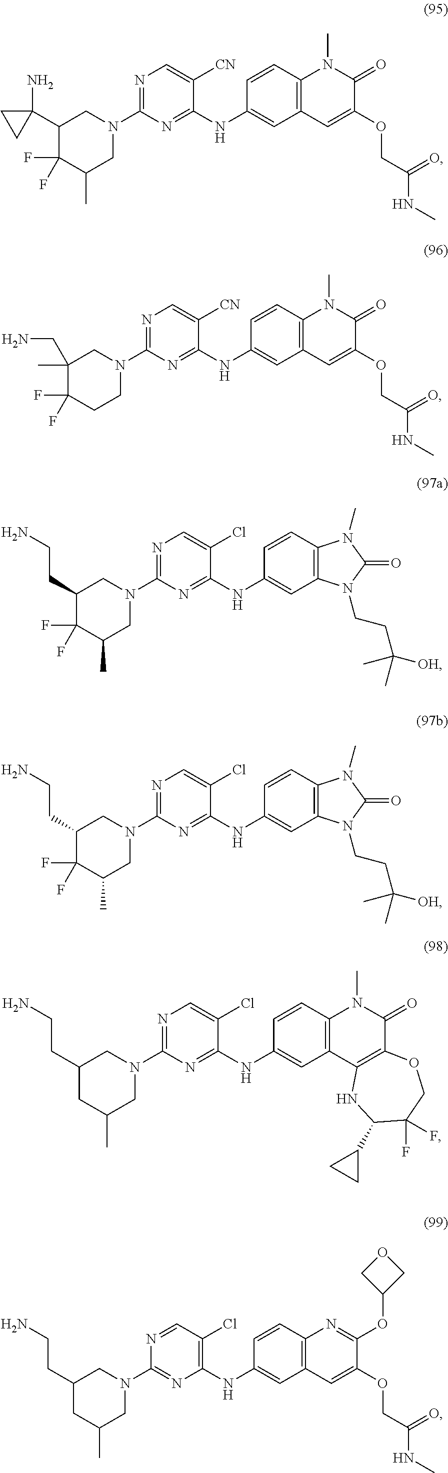 [SMILES: CNC(=O)COc1cc2cc(Nc3nc(N4CC(C)C(F)(F)C(C5(N)CC5)C4)ncc3C#N)ccc2n(C)c1=O ; CNC(=O)COc1cc2cc(Nc3nc(N4CCC(F)(F)C(C)(CN)C4)ncc3C#N)ccc2n(C)c1=O ; C[C@@H]1CN(c2ncc(Cl)c(Nc3ccc4c(c3)n(CCC(C)(C)O)c(=O)n4C)n2)C[C@H](CCN)C1(F)F ; C[C@H]1CN(c2ncc(Cl)c(Nc3ccc4c(c3)n(CCC(C)(C)O)c(=O)n4C)n2)C[C@@H](CCN)C1(F)F ; CC1CC(CCN)CN(c2ncc(Cl)c(Nc3ccc4c(c3)c3c(c(=O)n4C)OCC(F)(F)[C@H](C4CC4)N3)n2)C1 ; CNC(=O)COc1cc2cc(Nc3nc(N4CC(C)CC(CCN)C4)ncc3Cl)ccc2nc1OC1COC1] 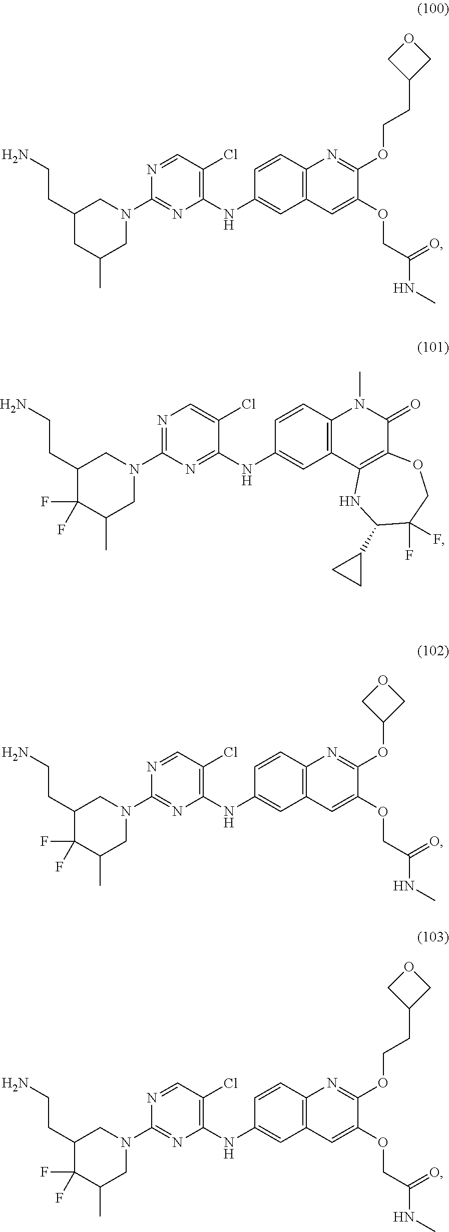 [SMILES: CNC(=O)COc1cc2cc(Nc3nc(N4CC(C)CC(CCN)C4)ncc3Cl)ccc2nc1OCCC1COC1 ; CC1CN(c2ncc(Cl)c(Nc3ccc4c(c3)c3c(c(=O)n4C)OCC(F)(F)[C@H](C4CC4)N3)n2)CC(CCN)C1(F)F ; CNC(=O)COc1cc2cc(Nc3nc(N4CC(C)C(F)(F)C(CCN)C4)ncc3Cl)ccc2nc1OC1COC1 ; CNC(=O)COc1cc2cc(Nc3nc(N4CC(C)C(F)(F)C(CCN)C4)ncc3Cl)ccc2nc1OCCC1COC1]
or a pharmaceutically acceptable salt or stereoisomer thereof.

48. A pharmaceutical composition, comprising a therapeutically effective amount of the compound or pharmaceutically acceptable salt or stereoisomer of claim 1, and a pharmaceutically acceptable carrier.

49. The pharmaceutical composition of claim 48, which is in the form of a liquid or a solid.

50. A method of treating a disease or disorder characterized by aberrant B-cell lymphoma 6 (BCL6) activity, comprising administering to a subject in need thereof a therapeutically effective amount of the compound or a pharmaceutically acceptable salt or stereoisomer thereof of claim 1.

51. The method of claim 50, wherein the disease or disorder is a lymphoid malignancy.

52. The method of claim 51, wherein the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

53. The method of claim 50, further comprising administering an additional anti-cancer agent.

54. The method of claim 53, wherein the additional anti-cancer agent is an enhancer of zeste homolog 2 (EZH2) inhibitor.
